(19) United States
(12) Patent Application Publication
(10) Pub. No.: US 2026/0335367 A1
(43) Pub. Date: Oct. 29, 2026

(54) DONOR-SPECIFIC TOLERANCE-INDUCING REGIMENS FOR XENOTRANSPLANTATION

(71) Applicant: Revivicor, Inc., Blacksburg, VA (US)

(72) Inventors: David Ayares, Blacksburg, VA (US); Lars Burdorf, Blacksburg, VA (US)

(73) Assignee: Revivicor, Inc., Blacksburg, VA (US)

(21) Appl. No.: 19/188,583

(22) Filed: Apr. 24, 2025

Related U.S. Application Data
(60) Provisional application No. 63/638,010, filed on Apr. 24, 2024.

(51) Int. Cl. A61K 35/26; A01K 67/0276; A61B 34/30; A61K 35/22; A61K 35/42; A61K 39/00; A61P 37/06; C12N 5/078

(52) U.S. Cl. CPC A61K 35/26; A01K 67/0276; A61K 35/22; A61K 35/42; A61K 39/001; A61P 37/06; C12N 5/065; A01K 2217/054; A01K 2227/108; A01K 2267/025; A61B 34/30; A61B 2034/302; A61K 2039/5158; A61K 2039/577

(57) ABSTRACT

The present disclosure provides methods of generating composite thymic kidney organoids for inducing immune tolerance to a renal xenograft in a subject in need thereof. Specifically, methods for inducing donor-specific tolerance to a recipient of a kidney organ transplant, by subcapsularly injecting an autologous or an allogeneic macerated thymus (e.g., engineered or modified and/or cultured) into a kidney of a transgenic donor porcine animal.

### CROSS-REFERENCE TO RELATED APPLICATIONS

This application claims the benefit of priority to U.S. Provisional Application No. 63/638,010, filed Apr. 24, 2024, the content of which is hereby incorporated by reference in its entirety for any and all purposes.

### TECHNICAL FIELD

The present disclosure relates generally to xenotransplantation therapies, and more particularly to methods of generating composite thymic kidney organoids for inducing immune tolerance to a renal xenograft in a subject in need thereof.

### BACKGROUND

Xenotransplantation (transplant of organs, tissues and cells from a donor of a different species) could effectively address the shortage of human donors. While advantageous in many ways, xenotransplantation creates a more complex immunological scenario than allotransplantation. The most profound barrier to xenotransplantation is the rejection of the grafted organ by a cascade of immune mechanisms, divided into three phases: hyperacute rejection (HAR), acute humoral xenograft rejection (AHXR), and T-cell mediated cellular rejection. HAR is a very rapid event that results in irreversible graft damage and loss within minutes to hours following graft reperfusion.

Considerable effort has been directed at addressing the immune barrier posed by xenotransplantation through genetic modification of the donor animal. The most commonly used donor animals are pigs. Pigs have been the focus of most research in xenotransplantation because pigs share many anatomical and physiological characteristics with humans. Furthermore, pigs have relatively short gestation periods and can be bred in pathogen-free environments. Pigs also do not present the same ethical issues associated with most animal research (e.g., primates) because pigs are commonly used as a food source by humans.

While engineering transgenic animals comprising multiple genetic modifications was shown to be necessary for the reduction of xenoantibody responses following a xenotransplant (e.g., HAR and AHXR), the immune response to xenografts (e.g., T-cell mediated cellular rejection) remains a powerful limiting factor to xenotransplantation.

Accordingly, there is a need for additional methods for mitigating the risk of host T-cell-mediated immune activation in response to donor neoantigens; and methods for improving the functional efficacy of xenogeneic thymic transplantation. The present disclosure addresses this need.

### SUMMARY OF THE INVENTION

One aspect of the present disclosure provides a method of inducing a transplant tolerance, the method comprising, consisting of, or consisting essentially of: (a) macerating a thymic tissue obtained from a donor animal; (b) injecting the macerated thymic tissue into a sub-renal capsule of the donor animal; (c) maturing the injected macerated thymic tissue into a composite renal subcapsular thymic lymphoid tissue; and (d) generating a composite thymo-kidney organ. In some embodiments, the donor animal is thymectomized prior to the injection. In some embodiments, the donor animal is partially thymectomized using a blunt and sharp dissection.

One aspect of the present disclosure provides a method of inducing a transplant tolerance, the method comprising, consisting of, or consisting essentially of: (a) macerating a thymic tissue obtained from a donor animal; (b) injecting the macerated thymic tissue into a kidney capsule or a lung of the donor animal or a first recipient; (c) maturing the injected macerated thymic tissue into a composite renal subcapsular thymic lymphoid tissue or a composite pulmonary thymic lymphoid tissue; and (d) generating a composite thymo-kidney organ or a composite thymo-lung organ. In some embodiments, the donor animal or the first recipient is thymectomized prior to the injection.

Another aspect of the present disclosure provides a method of inducing a transplant tolerance, the method comprising, consisting essentially of, or consisting of: (a) macerating a thymic tissue obtained from a donor animal; (b) culturing the macerated thymic tissue in a culture regimen for about 1 day to about 30 days; (c) injecting the macerated thymic tissue into a sub-renal capsule or a quadriceps muscle of the donor animal or a first recipient; (d) maturing the injected macerated thymic tissue into a composite renal subcapsular thymic lymphoid tissue or a composite musculo-thymic lymphoid tissue; and (e) generating a composite thymo-kidney organ or a thymo-muscle organ. In some embodiments, the donor animal or the first recipient is thymectomized prior to the injection. In some embodiments, the donor animal is partially thymectomized using a blunt and sharp dissection.

Another aspect of the present disclosure provides a method of inducing a transplant tolerance, the method comprising, consisting essentially of, or consisting of: (a) macerating a thymic tissue obtained from a donor animal; (b) culturing the macerated thymic tissue in a culture regimen for about 1 day to about 30 days; (c) injecting the macerated thymic tissue into a kidney capsule, a lung, or a quadriceps muscle of the donor animal or a first recipient; (d) maturing the injected macerated thymic tissue into a composite renal subcapsular thymic lymphoid tissue, a composite pulmonary thymic lymphoid tissue or a composite musculo-thymic lymphoid tissue; and (e) generating a composite thymo-kidney organ, a composite thymo-lung organ or a thymo-muscle organ. In some embodiments, the donor animal or the first recipient is thymectomized prior to the injection.

In some embodiments of any of the methods disclosed herein, the macerated thymic tissue is injected subcapsularly into the kidney, or into a lung parenchyma or a lung pleura of the donor animal or the first recipient.

In some embodiments, the macerated thymic tissue or the thymus tissue pieces injection is repeated at least twice, at least 3 times, at least 4 times, at least five times or more on other locations of the kidney.

In some embodiments, the macerated thymic tissue or the thymus tissue pieces injection is repeated at least twice, at least 3 times, at least 4 times, at least five times or more on other locations of the kidney.

In some embodiments, the method is a robotic auto thymokidney transplantation (TKT). In that embodiment, the macerated thymic tissue is injected at one location, 2 locations, 3 locations, 4 locations, 5 locations, 6 locations, or more locations of the same kidney using a single surgical incision. In some embodiments, the injection of the macerated thymic tissue or the thymus tissue pieces can be repeated at least twice, at least 3 times, at least 4 times, at least five times or more on other locations of the kidney.

In some embodiments, the robotic auto thymokidney transplantation (TKT) comprises a da Vinci® Xi™ Surgical System or a da Vinci SP® Surgical System. In some embodiments, the robotic auto thymokidney transplantation (TKT) comprises a da Vinci® Xi™ Surgical System.

In some embodiments of any of the methods disclosed herein, the macerated thymic tissue or thymic tissue is injected into a lung of a donor animal or a first recipient. In some embodiments, the macerated thymic tissue or thymic tissue is injected into an intrapulmonary cavity. In some embodiments, the intrapulmonary cavity can contain 3-5 g of macerated thymic tissue or thymic tissue. In some embodiments, the intrapulmonary cavity can contain about 1 g, about 1.5 g, about 2.5 g, about 3 g, about 3.5 g, about 4 g, about 4.5 g, about 5 g, or more grams of macerated thymic tissue or thymic tissue. In some embodiments, the intrapulmonary cavity can contain 3.5 g of macerated thymic tissue or thymic tissue. In some embodiments, the macerated thymic tissue or thymic tissue comprises about one-inch (1″) long and about quarter-inch (¼″) diameter pieces.

In some embodiments, the macerated thymic tissue or thymic tissue is injected into a lung parenchyma or pleura. In some embodiments, the macerated thymic tissue or thymic tissue is injected into a lung intra-parenchymal cavity. In some embodiments, the macerated thymic tissue or thymic tissue is injected into a lung sub-pleural cavity. In some embodiments, a thymolung is generated.

Another aspect of the present disclosure provides a method of inducing a transplant tolerance, the method comprising, consisting of, or consisting essentially of: (a) macerating a thymic tissue obtained from a donor animal; (b) injecting the macerated thymic tissue into a sub-renal capsule of the donor animal; (c) maturing the injected macerated thymic tissue into a composite renal subcapsular thymic lymphoid tissue; (d) generating a composite thymo-kidney organ; and (e) comprises transplanting the composite thymus-kidney organ in a subject in need of a kidney transplant.

Another aspect of the present disclosure provides a method of inducing a transplant tolerance, the method comprising, consisting essentially of, or consisting of: (a) macerating a thymic tissue obtained from a donor animal; (b) culturing the macerated thymic tissue in a culture regimen for about 1 day to about 30 days; (c) injecting the macerated thymic tissue into a sub-renal capsule or a quadriceps muscle of the donor animal or a first recipient; (d) maturing the injected macerated thymic tissue into a composite renal subcapsular thymic lymphoid tissue or a composite musculo-thymic lymphoid tissue; (e) generating a composite thymo-kidney organ or a thymo-muscle organ; and (f) transplanting the composite thymus-kidney organ in a subject in need of a kidney transplant.

In some embodiments of any of the methods disclosed herein, the composite thymus-kidney organ induces thymopoiesis in the subject (e.g., the transplanted subject) within about 1 month, about 2 months, about 3 months, about 4 months, about 5 months, about 6 months, about 7 months, about 8 months, about 9 months, about 10 months, about 11 months, or about 12 months.

In some embodiments of any of the methods disclosed herein, the macerated thymic tissue can be injected into the donor animal and the composite thymo-kidney organ or the thymo-muscle organ can be an autologous composite thymo-kidney organ.

In some embodiments, macerating comprises slicing the donor thymic tissue into about 0.1 mm, about 0.2 mm, about 0.3 mm, about 0.4 mm, about 0.5 mm, about 0.6 mm, about 0.7 mm, about 0.8 mm, about 0.9 mm, about 1.2 mm, about 1.3 mm, about 1.4 mm, or about 1.5 mm slices.

In some embodiments of any of the methods disclosed herein, the subcapsular injection is performed by a method comprising the steps of: (a) placing the donor animal in a lateral position; (b) introducing a horizontal or a vertical incision extending from the last rib into the paralumbar fossa and over the kidney; (c) mobilizing the caudal pole and lateral aspect of the kidney; (d) exposing the kidney within a Gerota's fascia; (e) introducing a small incision in the kidney capsule; (f) separating the renal capsule from the renal cortex to generate a pocket; (g) injecting the macerated thymic tissue into the pocket; (h) closing the kidney capsule; and (i) closing the horizontal or the lateral incision. In some embodiments, the kidney capsule is closed with a Prolene 6-0 suture, or the kidney capsule incision is cauterized.

In some embodiments, the horizontal or vertical incision is about 5 cm to about 8 cm.

Ins some embodiments of any of the method disclosed herein, (a) the macerated thymic tissue is injected subcapsularly toward the cranial pole of the kidney capsule; and/or (b) the injected macerated thymic tissue covers one surface of the caudal renal pole.

In some embodiments, the subcapsular injection is performed using a robot (e.g., da Vinci® Xi™ Surgical System or da Vinci SP® Surgical System), a silicone catheter, a blunted needle, or a modified pipette.

In some embodiments of any of the methods disclosed herein, the macerated thymic tissue is cultured for about 5 to about 7 days, about 5 to about 12 days, about 5 to about 9 days, about 10 to about 20 days, about 5 to about 12 days, about 5 to about 15 days, or about 10 to about 20 days. In some embodiments, the thymic tissue is macerated for at least about 15 days.

In some embodiments, culturing comprises changing a culture medium daily. In some embodiments, culturing comprises contacting the thymus tissue with an agent selected from the group consisting of 2-deoxyguanosine (2DG), cyclosporine A, hydrocortisone, notch inhibitors, ABT-737, and 2D glucose, or a combination of 2 or more thereof.

In some embodiments, culturing comprises contacting the thymic tissue with 2-DG. In some embodiments, culturing comprises contacting the thymic tissue with about 1 nM to about 500 nM 2-DG, about 25 nM to about 250 nM 2-DG, about 50 nM to about 150 nM 2-DG, about 50 nM to about 100 nM 2-DG, about 100 nM to about 300 nM 2-DG, or about 250 nM to about 500 nM 2-DG for about 2 hours to about 24 hours.

In some embodiments, culturing comprises contacting the thymic tissue with about 100 nM 2-DG for about 15 hours. In some embodiments, the culture regimen depletes the thymus tissue of donor thymocytes; and/or maintains (e.g., enriches) thymic stromal components.

In some embodiments, culturing comprises suspending the macerated thymic tissue in an extracellular matrix composition or a biomaterial prior to injecting the macerated thymic tissue into the sub-renal capsule or the quadriceps muscle of the donor animal or the first recipient.

In some embodiments, the extracellular matrix composition comprises a collagen matrix, a laminin matrix, a fibronectin matrix, a collagen/laminin matrix, or Matrigel™.

In some embodiments of any of the methods disclosed herein, culturing further comprises co-culturing the thymic tissue with isolated thymic epithelial cells (TECs) obtained from the subject to be transplanted. In some embodiments, the thymic tissue and the isolated thymic epithelial cells are injected into the sub-renal capsule or the quadriceps muscle of the donor animal or the first recipient.

Another aspect of the methods disclosed herein comprises maturing the macerated thymic tissue. In some embodiments, the injected macerated thymic tissue matures for about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, or about 20 weeks.

In some embodiments of any of the methods disclosed herein, the donor or the first recipient is a pig. In some embodiments, the donor or the first recipient is a transgenic pig that: (a) lacks expression of a functional gene selected from an alpha 1,3 galactosyltransferase, a growth hormone receptor (GHR), a β-1,4-N-acetyl-galactosaminyltransferase 2 (β4GalNT2), a cytidine monophosphate-N-acetylneuraminic acid hydroxylase (CMAH), or a combination thereof; and (b) expresses at least six exogenous transgenes selected from an anticoagulant transgene, a cytoprotective transgene, an immunosuppressant transgene, or a complement inhibitor transgene, or a combination thereof.

In some embodiments, (a) the cytoprotective transgene is selected from the group consisting of heme oxygenase 1 (HO-1), A20, FAT-1, and soluble tumor necrosis factor-alpha (TNF-alpha), or a combination thereof; (b) the immunosuppressant transgene is selected from the group consisting of Cytotoxic T-Lymphocyte-Associated Protein 4 (CTLA4), cluster of differentiation 47 (CD47), and Class II transactivator-DN (CIITA-DN) or a combination thereof; (c) the anticoagulant transgene is selected from the group consisting of endothelial protein C receptor (EPCR), thrombomodulin, CD39, hirudin, and Tissue factor pathway inhibitor (TFPI), or a combination thereof; and/or (d) the complement inhibitor is selected from the group consisting of CD46, DAF (CD55), CD59, and CR1, or a combination thereof.

In some embodiments of any of the methods disclosed herein, the donor animal is about 1 month, about 2 months, about 3 months, about 4 months, about 5 months, about 6 months, about 7 months, about 8 months, about 9 months, about 10 months, about 11 months, about 12 months, about 24 months, about 36 months, about 48 months, about 60 months, about 72 months, about 84 months, about 96 months, about 108 months, or about 120 months.

### BRIEF DESCRIPTION OF THE DRAWINGS

FIG. 1 shows a workflow for the process of inducing donor-specific immune tolerance to a xenotransplant in a subject (e.g., a human). The first step (A) can include generating a genetically modified pig comprising multiple genetic modifications (e.g., gene knockout and expression of at least one exogenous transgene). The second step (B) can include methods of generating composite thymic kidney organoids (UThymoKidney) from the genetically modified pig. The third step (C) can include the maturation of the composite thymic kidney organoids. The last step (D) can include the procurement and xenotransplant of the UThymoKidney to a subject in need thereof.

FIGS. 2A-B show schematics of multigene vectors used to engineer the genetically modified pig and the exogenous transgenes that were expressed in the genetically modified pig.

FIGS. 3A-G show schematic representations of the targeted integration of multicistronic vectors comprising six human transgenes (6-gene vectors) into exemplary loci and embodiments of functional knockouts of porcine genes (e.g., GGTA1, CMAH, B4GalNT2, and GHR) for producing the genetically modified pig.

FIG. 4 shows a schematic representation of the B217 vector and a schematic representation of the production of a genetically modified animal comprising at least 10 modifications (i.e., 10GE pigs).

FIGS. 5A-C show a western blot analysis of various tissues from a genetically modified animal expressing a multitransgenic vector and demonstrating the consistent protein expression of the transgenes encoded by the B200, B201, B202, and B209 in heart (FIG. 5A), lung FIG. 5B) and kidney (FIG. 5C). Transgenic proteins from transgenic tissue samples had expected molecular weight by western blot and showed substantially similar expression levels across tissues.

FIGS. 6A-E show representative images of immunohistochemical stainings of porcine lung, heart, and kidney tissues expressing human transgenes encoded by vectors B200 (FIG. 6A), B201 (FIG. 6B) and B202 (FIG. 6C), and of porcine heart and kidney tissues expressing human transgenes encoded by vectors B209 (FIG. 6D) and B212 (FIG. 6E). Transgenic proteins from transgenic tissue samples showed substantially similar expression levels across tissues.

FIG. 7 shows representative images of immunohistochemical stainings of porcine kidneys expressing the B201 transgene in porcine kidney transplanted into a baboon; and demonstrates the stable expression of all human transgenes encoded by the B201 vector. Porcine kidneys were obtained and stained immediately post-mortem from a baboon on Day 120 post-transplant.

FIGS. 8A-B show the transplantation of thymic tissue pieces into the left upper lobe of a lung parenchyma and pleura. FIG. 8A shows a lung intra-parenchymal cavity containing thymus tissue and the airtight cavity stapler closure from the first experiment. FIG. 8B shows a lung sub-pleural cavity containing thymus tissue and the airtight cavity stapler closure from the second experiment.

FIG. 9 shows an image of a peritoneum incision following a Robotic auto ThymoKidney Transplantation (TKT) using a da Vinci® Xi™ Surgical System and further illustrates the thymus under a kidney capsule and the closed kidney capsule incision post TKT.

FIGS. 10A-D show T cell recovery in a 54-year-old patient who received a porcine thymokidney transplant following thymoglobulin induction. FIG. 10A shows peripheral blood T cell concentrations, frequency of naïve CD4 cells (CD45RA⁺ CCR7⁺) and frequency of CD4 cells that are CD45RA⁺/CCR7⁺/CD31⁺ recent thymic emigrants (RTEs) over time. FIG. 10B shows H&E staining and pan-cytokeratin immunohistochemistry demonstrating lymphoid aggregation and in formalin-fixed, paraffin-embedded (FFPE) porcine thymic tissue explanted at post-operative day (POD) 47. FIG. 10C shows flow cytometric analysis of human thymocytes as positive control and the patient's porcine thymus sample from thymokidney explant. Human CD45⁺ cells were gated, followed by gating of “CD4^{neg}CD8^{neg} “double negative” cells. The human CD45 and CD34 gates were selected on the basis of “fluorescence minus one” stains. The double negative population in the porcine thymus included subsets expressing CD5, CD7, CD1a, and CD34, which was consistent with T cell progenitor populations found in a normal human thymus. FIG. 10D shows mixed lymphocyte responses of purified patient T cells to donor pig (3^{rd} party pig or 3^{rd} Pig), allogeneic human (3^{rd} Human) and no stimulator (Responder Only) at the indicated timepoints.

FIGS. 11A-D show the reconstitution of the thymic tissue under a kidney capsule (UThymo™ kidney (i.e., thymokidney) and demonstrate that the UThymo™ kidney achieved immune tolerance. FIG. 11A shows a representative image of reconstituted thymic tissue adjacent to normal appearing renal tissue from a wedge biopsy of the non-implanted UThymo™ kidney. FIG. 11B shows thymopoeisis 47 days after UThymo™ kidney pig-to-human xenotransplantaIon. FIG. 11C shows T-cell enrichment for RTEs following UThymo™ kidney pig-to-human xenotransplantaIon. FIG. 11D shows MLR responses of purified patient T-cells to UThymo™ donor pig, 3^{rd} party pig, allogeneic human cells, and no stimulator. Unresponsiveness to the UThymo™ donor pig was achieved by POD47 and persisted through the study conclusion.

FIGS. 12A-C show the characterization of healthy donor xenoreactive T cell (XDRTCCs) repertoire in vitro indirectly and directly. FIG. 12A, upper panel shows a method for direct xenogeneic mixed lymphocyte reaction (MLR). Porcine DC were co-cultured with CFSE-labeled purified human T cells. After 6 days, proliferated T cells, identified as CFSE^{low}, were sorted by flow cytometry, genomic DNA was extracted, and high throughput TCRB CD3 sequencing was carried out. FIG. 12A, lower panel shows a method for indirect xenogeneic MLR. Pig PBMCs were irradiated with 100 Gy to induce apoptosis and loaded into differentiated, sorted human macrophages. Loaded macrophages were MACS sorted for CD14 expression and combined with CFSE-labeled purified T cells. Proliferated CFSE^{low} T cells were sorted by flow cytometry, genomic DNA was extracted, and high throughput TCRB CDR3 sequencing was carried out. FIG. 12B shows direct and indirect MLRs that induced proliferation of both CD4 and CD8 cells. FIG. 12C shows the clonal frequency plots of divided T cells following high-throughput TCR sequencing of the CD4 and CD8 response to indirect (y axis) and direct (x axis) stimulation by porcine antigens. Clonotypes that were unique to one pathway appear along the y or x axis (for indirect and direct respectively). Clonotypes found in both sequence sets are depicted within the yellow-shaded area.

FIGS. 13A-D show an overview of long-term thymokidney xenotransplant, sampling, and TCRB CDR3 sequencing of peripheral blood T cell DNA over time. FIG. 13A shows a schematic overview of the xenotransplantation experiment. Blood, kidney, and lymphocele samples were obtained from the decedent human and donor pig pre- and post-transplantation. T cells were sorted from unstimulated (unstim) populations or from CFSE^{low} cells in MLRs. FIG. 13B shows clonal expansion of circulating CD4 and CD8 T cells over time. CD4 and CD8 T cells were not separated in pre-transplant FACS sort. Fisher's exact test was used to compare proportion of hyperexpanded clones compared to pre-Tx baseline, ****P<0.0001. FIG. 13 C shows cumulative frequency of clones shared between timepoints. The reference population on the x-axis is the population whose cumulative frequency is plotted, and the overlap frequency is shown for the populations on the y-axis. The pre-transplant repertoire has been included by estimating the number of CD4 and CD8 templates using flow cytometric data (FIG. 31). FIG. 13D shows frequency of the top 5 CD8 clones from every sequenced post-operative timepoints. Each color represents the frequency of a specific clonotype at that timepoint. Some of the top 5 clonotypes are shared across the timepoints, resulting in fewer than 15 unique clones.

FIGS. 14A-C show the number of unique clones, templates and diversity metrics of post-operative peripheral blood T cell repertoire. FIG. 14A shows the number of unique clones and templates in unstimulated TCRB sequencing samples from peripheral blood. FIG. 14B shows the proportion of clonotypes needed to account for 20% of total TCRB CDR3 templates (R20) was calculated for both CD4 and CD8 repertoires at each timepoint. An R20 of 0.20 represents the maximum value, indicating a maximally diverse repertoire with no clonotype expansion. FIG. 14C shows that clonality was measured for the different repertoires at each post-operative timepoint. A clonality of 0 indicates a maximally diverse repertoire where all clonotypes are unique, while a clonality of 1 indicates that all cells in the repertoire share the same clonotype.

FIGS. 15A-E show proportional Venn diagrams summarizing MLRs and repertoire overlap analyses. FIG. 15A shows the percentage of proliferating CD4 and CD8 T cells against donor pig antigens at pre-transplantation. FIG. 15B shows pre-transplant CD4 XDRTCC overlap between PBMC direct and indirect responses and lymph node (LN) direct responses. FIG. 15C shows pre-transplant CD8 XDRTCC overlap between PBMC direct and indirect responses and LN direct responses. FIG. 15D shows overlap between pre-transplant, POD28 and POD49 CD4 XDRTCCs. Pre-transplant CD4 XDRTCCs from direct and indirect PBMC and direct LN responses are combined into one repertoire composed of all unique clones. FIG. 15E shows overlap between pre-transplant, POD28 and POD49 CD8 XDRTCCs. Pre-transplant CD8 XDRTCCs from direct and indirect PBMC and direct LN responses are combined into one repertoire composed of all unique clones.

FIGS. 16A-F show that XDRTCCs demonstrated selective expansion and enrichment in the blood over time. FIGS. 16A-B show cumulative frequency of CD4 (FIG. 16A) and CD8 (FIG. 16B) XDRTCCs and non-XDRTCCs defined in pre-transplant MLR at each timepoint. FIG. 16C shows designation of the top 5 expanded CD8 clones (as described in FIG. 13D) according to XDRTCC or non-XDRTCC status defined pre-transplant, at each timepoint post-transplant. b shows the cumulative frequency fold change of both CD4 and CD8 post-transplant XDRTCCs as normalized to their pre-transplant baseline. The formula is shown. FIG. 16E shows the relative clonal detection rate of both CD4 and CD8 XDRTCCs compared to non-XDRTCCs identified pre-transplant. The formula is shown. FIG. 16F shows the relative template detection rate of both CD4 and CD8 XDRTCCs compared to non-XDRTCCs identified pre-transplant. The formula is shown. For FIGS. 16D, E and F, a dotted line is plotted at y=1 to represent the cutoff between XDRTCC enrichment (>1) or depletion (<1). The statistics establish significance between the numerator and denominator in every case (post-transplant to pre-transplant XDRTCC cumulative frequency for FIG. 16D, and XDRTCC to non-XDRTCC enrichment for FIGS. 16E and F). *P<0.05, **P<0.01, ***P<0.001, ****P<0.0001, by Z test.

FIGS. 17A-E show XDRTCC relative metrics. FIG. 17A shows origin of circulating CD4 XDRTCC templates (left panel) and unique clones (right panel). “Undefined” indicates that the clone was found in the indirect as well as in any direct MLRs or was only identified in the POD49 PBMC-on-PBMC MLR. The number of unique clones comprising every category is shown in brackets. FIG. 17B shows origin of circulating CD8 XDRTCC templates (left panel) and clones (right panel). “Undefined” indicates that the clone was found in the indirect as well as in any direct MLRs or was only identified in the POD49 PBMC-versus-PBMC MLR. The number of unique clones comprising every category is shown in brackets.

FIG. 17C shows fractions of total post-transplant peripheral blood CD4 clones mappable to XDRTCC and non-XDRTCC clone sets. Pre-transplant non-XDRTCCs comprised almost 100% of clones in pre-transplant unstimulated peripheral blood and is therefore not shown. Statistical analysis was done using the Z-test.

FIG. 17D shows fractions of total post-transplant peripheral blood CD8 clones mappable to XDRTCC and non-XDRTCC clone sets. Pre-transplant non-XDRTCCs comprised almost 100% of clones in pre-transplant unstimulated peripheral blood and is therefore not shown. Statistical analysis was done using the Z-test.

FIG. 17E shows average numbers of nucleotide sequences encoding each unique amino acid sequence (nucleotide-per-aa) for XDRTCCs and non-XDRTCCs found in the pre-transplant and post-transplant peripheral blood. “Remainder” indicates clones that were identifiable as neither XDRTCCs nor non-XDRTCCs post-transplant. Statistical analysis was done using the Z-test. *P<0.05, **P<0.01, ***P<0.001, ****P<0.0001, by Z test.

FIGS. 18A-B show tracking of XDRTCCs in bulk TCRB RNA sequencing data from kidney biopsies. FIG. 18A shows a Table summarizing the bulk RNA sequencing data, displaying the number of unique TCRBs identified in each biopsy (upper row), along with the number and frequencies of unique CD4 and CD8 XDRTCCs found in each biopsy (second and third rows, respectively). FIG. 18B shows frequencies of five circulating immunodominant CD8 XDRTCCs, previously identified in FIGS. 2D and 3C, within the POD45 biopsy.

FIG. 19 shows tracking XDRTCCs across locations over time. The location of different clonotypes is shown over time for the top 5 circulating immunodominant CD8 XDRTCCs of FIGS. 13D and 16C. Several of these dominant clones can be traced preceding rejection at POD14 through to POD61, at study termination. The template frequency of a given clonotype is shown for POD 28, POD33, and POD49 in the blood, for which high throughput bulk TCR sequencing is available.

FIGS. 20A-C show graphs distinguishing pig and human cells in single cell RNA sequencing. FIG. 20A shows the numbers of pig and human transcripts per cell for leukocyte samples isolated from kidney biopsies and the POD14 lymphocele, with the majority of cells containing primarily pig (y axis) or human cells (x axis) only. FIG. 20B shows sequencing data were run in CellRanger using a human only reference generating the UMAP shown. FIG. 20C shows pig cells (along y axis) and their associated barcodes in panel A were identified and removed from the UMAP in panel B, resulting in the UMAP shown, containing only human cells.

FIG. 21-B show single-cell RNA sequencing of graft leukocytes demonstrating the upregulation of cytotoxic populations during rejection. FIG. 21A shows an integrated UMAP showing single-cell RNA sequencing results on hCD45⁺ sorted cells from POD14 kidney biopsy (POD14K), POD14 lymphocele (POD14L), POD28 kidney biopsy (POD28K), POD33 kidney biopsy (POD33K), and POD61 kidney biopsy (POD61K). Cluster numbering is shown. When split by POD, specific clusters predominate in specific samples. For example, clusters 0, 1, 6 and 9 are primarily found in POD14L, whereas clusters 2, 3, 4, 5, and 12 are predominantly found in the POD33 kidney specimen. FIG. 21B shows expression of T cell, effector T and NK cell, NK cell, and APC-associated RNA species in each cluster and specimen in FIG. 21A.

FIGS. 22A-C show the frequency of different cell types in the lymphocele and kidney xenograft. FIG. 22A shows UMAP with different cell populations highlighted with their corresponding definitions below. All definitions were mutually exclusive. FIG. 22B shows the proportion of each cell type at different PODs. FIG. 22C shows the frequency of each population per cluster.

FIGS. 23A-D show XDRTCCs demonstrating a cytotoxic phenotype, particularly during rejection at POD33. FIG. 23A shows the distribution of XDRTCCs (shown as iii) across POD14K, POD14L, POD28K, POD33K, and POD61K samples and demonstrates that the majority are found in POD14L and POD33K samples. FIGS. 23B-C show differential gene expression in clusters 3 and 5, containing the most XDRTCCs, demonstrated upregulation of effector cell-associated genes in both clusters 3 (GZMB, GZMH, FASLG) and 5 (GZMK, CCL5, EOMES, CXCR3). In addition, cluster 3 demonstrated upregulation of γδ T cell genes TRGV9, TRGC, and NK cell-associated gene NCR3, whereas cluster 5 demonstrated upregulation of adaptive T cell genes including CD8B and TRBC2. FIG. 23D shows KEGG pathway analysis of the top differentially expressed genes demonstrated NK cell-mediated cytotoxicity as the top pathway for cluster 3.

FIGS. 24A-D show XDRTCCs and non-XDRTCCs are detected in the lymphocele and kidney. FIGS. 24A-B show that XDRTCCs were found at highest frequency in clusters 5 and 3. FIGS. 24C-D show that XDRTCCs detected in the POD14L (clusters 0, 1, 6) and POD33 kidney biopsy (cluster 3) were primarily CD8 T cells from the direct pathway.

FIG. 25 shows XDRTCC clonotype sharing between the lymphocele and kidney biopsy rejection specimen. XDRTCCs (labeled in darker tone) with the indicated TCRB CDR3 sequences were detected in both the perinephric lymphocele at POD14 and within the kidney biopsy at POD33.

FIG. 26 shows that Clusters 3 and 5 containing most XDRTCCs expressed markers of T cell activation and cytotoxicity. Cluster 3 expressed increased markers of T cell cytotoxicity (GZMB, GZMA, PRF1) as well as increased markers of innate lymphocyte cytotoxicity (GNLY, KLRD1, KLRC1) compared to Cluster 5. *P<0.05, **P<0.01, ***P<0.001, ****P<0.0001, by Wilcoxon Rank Sum test.

FIGS. 27A-E show that during rejection, γδ T cells and NK cells were found at high levels and express cytotoxic RNA profiles. FIG. 27A shows that Cluster 3 of FIG. 23A was subset and re-clustered to examine additional heterogeneity, separating out into 6 clusters. FIG. 27B shows that XDRTCCs were found in clusters 0, 2, and 5. XDRTCCs with identical clonotypes were annotated with identical color bars above the cluster identity metadata. Clusters containing XDRTCCs expressed some cytotoxic genes, though to a lesser extent than clusters 1, 3, 4, and 6. These other clusters also expressed high levels of γδ T cell and NK cell-associated genes. FIG. 27C shows αβ T cells segregate out from γδ and NK cells which co-cluster. FIG. 27D shows clusters 1, 3, 4, and 6 containing γδ and NK cells demonstrate higher levels of activation and cytotoxic genes than clusters 0, 2, and 4 containing XDRTCCs. FIG. 27E shows macrophages and dendritic cells (DCs) were predicted to interact with T cells by TIGIT, causing suppression, but NK and γδ T cells are predicted to interact via CD226, causing activation. *P<0.05, **P<0.01, ***P<0.001, ****P<0.0001, by Wilcoxon Rank Sum test.

FIGS. 28A-C show that αβ and γδ T cells displayed distinct RNA expression profiles. FIG. 28A shows that αβ and γδ T cells demonstrated distinct clustering on the UMAP, indicating differing RNA expression profiles. FIG. 28B shows the quantification of cell counts of each type per cluster. FIG. 28 C shows the quantification by percentage of each cell type in each cluster.

FIGS. 29A-D show that γδ T cells were found in multiple samples and demonstrated a strong cytotoxic transcriptional profile in POD33 kidney biopsy. FIG. 29A shows that γδ T cells were detected in POD14L and POD33 kidney. FIG. 29B shows that γδ T cells from POD14L and POD33K expressed markedly different TRGV genes. FIG. 29 C shows graphs comparing γδ T cells (defined as CD3E⁺ TRDC⁺) at POD14 and POD33, those at POD33 demonstrated higher activation of cytotoxic transcripts. FIG. 29D shows clusters 1, 3, 4, and 6 (upper half of UMAP) containing the majority of γδ T cells demonstrate high expression of a T cell cytotoxic signature from the Molecular Signatures Database (MSigDB) *P<0.05, **P<0.01, ***P<0.001, ****P<0.0001, by Wilcoxon Rank Sum test.

FIGS. 30A-D show cell-cell prediction analyses using CellChat package. FIG. 30A shows reference-based labeling of all single-cell RNAseq samples using the Single R package. FIG. 30B shows cluster labelling using the combination of manual annotation and reference-based prediction. The populations defined manually by gene expression showed high agreement with the predicted annotations in FIG. 30A. These cell labelings were used for the CellChat package. FIGS. 30C-D show cell-cell interaction prediction for the IFN-II and ICAM signaling pathways using CellChat package. Population labels are those defined in FIG. 30B. The cell populations expressing the ligand are on the left and those expressing the receptor are on the bottom.

FIGS. 31A-E show flow cytometric data of pre-transplant decedent PBMCs. FIG. 31A shows forward-side scatter profile of isolated PBMCs from decedent pre-transplant. FIG. 31B shows gating of single cells from FIG. 31A. FIG. 31C shows removal of dead cells from FIG. 31B. FIG. 31D shows gating of CD3⁺ cells from FIG. 31 C. FIG. 31E shows gating of CD4⁺ and CD8⁺ cells, which comprised 74.0% and 20.0% of CD3⁺ cells, respectively.

FIG. 32 shows Table 1. Table 1 shows the induction and maintenance immunosuppression. Induction immunosuppression consisted of rituximab, rabbit anti-thymocyte globulin (rATG), and methylprednisolone. Eculizumab was given from POD6 through POD42. Maintenance therapy included belatacept, tacrolimus (target 8-12 ng/mL), mycophenolate mofetil, and corticosteroids. In response to antibody-mediated rejection, treatment with plasmapheresis spanned PODs 34-42 and PODs 50-58. Pegcetacoplan was initiated on day 36 after plasma exchange, delivered as a continuous infusion and bolus after plasmapheresis. In response to incompletely resolved AMR detected on the POD49 biopsy, T cell re-depletion with additional rATG and a methylprednisolone pulse were administered.

FIG. 33 shows Table 2. Table 2 shows the workflow to identify XDRTCCs. (Step A) Number of unique CD4 and CD8 clones present in the divided subpopulations of the different MLRs performed. (Step B) Number of unique CD4 and CD8 clones remaining after removing the clones whose frequencies in the CFSE^{low} population were not at least twice that in the pre-transplant unstimulated population, the criterion for XDRTCC definition. (Step C) Number of total CD4 and CD8 XDRTCCs from the different MLRs. (Step D) Number of unique CD4 and CD8 XDRTCCs identified by our method.

FIG. 34 shows Table 3. Table 3 shows the number of isolated leukocytes from each sample. The indicated number of leucocytes was isolated from kidney biopsies/lymphocele sample and submitted for single-cell RNA sequencing.

FIG. 35 shows Table 4. Table 4 shows the Number of XDRTCCs per cluster. Clustering was performed according to the numbering in the reclustered UMAP in FIGS. 27A-D.

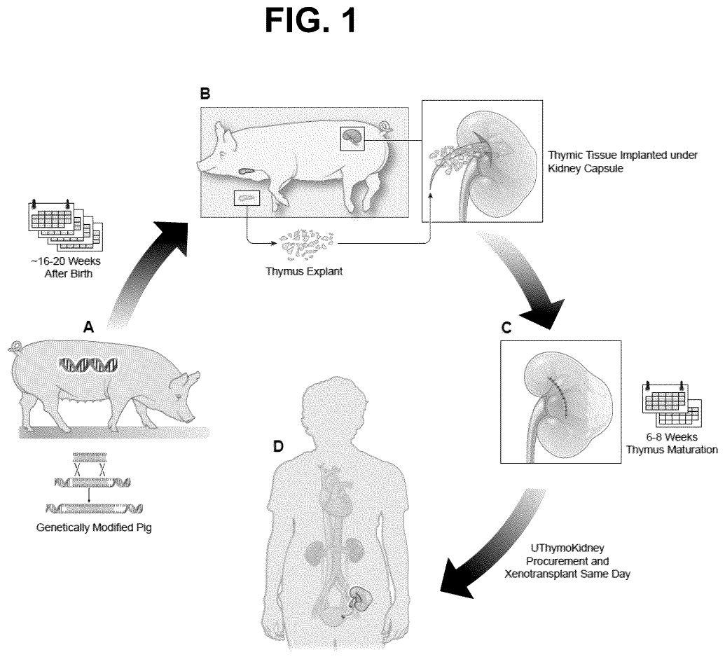

### DETAILED DESCRIPTION

### I. Overview

The immune response to xenografts remains a powerful limiting factor to xenotransplantation. Specifically, T cell-mediated immune activation can recognize other neoantigens found in the donor organ triggering a late allograft rejection. Tolerance induction was developed as an alternative means of enhancing xenograft survival. Tolerance induction appeared to be less toxic than traditional T-cell-suppression methods (e.g., immune suppression regimens). The two known approaches for inducing immune tolerance rely on the establishment of a mixture of host cells and donor cells that are capable of promoting the negative selection of newly developed host immune cells. The first approach is “Mixed chimerism.” “Mixed chimerism” relies on a bone marrow transplant from a donor animal to induce the negative selection of newly developed T cells, B cells and/or natural killer (NK) cells that are tolerant to the donor neoantigens in the recipient. The second approach is “Thymic transplantation.” Thymic transplantation relies on the infiltration of the donor's thymus or organoid thereof by the recipient's bone marrow-derived dendritic cells, which participate in the negative selection of newly developed T cells that are reactive to the recipient or donor neoantigens.

However, the activity of immune cells generated from thymic transplants were shown to be reduced when compared to the donor's or the recipient's immune cells. Accordingly, there is a need for additional methods for mitigating the risk of host T-cell-mediated immune activation in response to donor neoantigens; and methods for improving the functional efficacy of xenogeneic thymic transplantation. The present disclosure addresses this need.

### Thymolung Generation

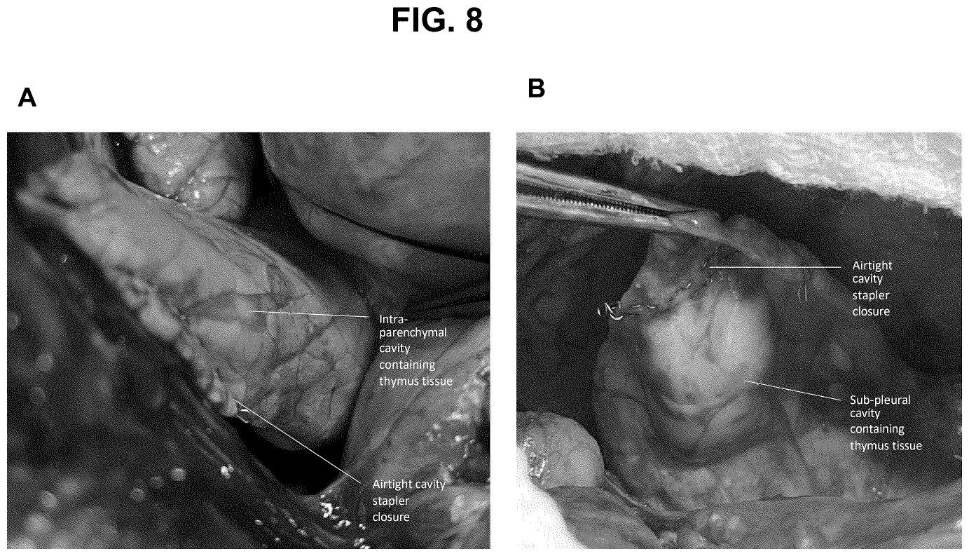

The present disclosure shows for the first time that transplantation of thymic tissue into the lung parenchyma or pleura is possible and feasible (FIGS. 8A and 8B). Using the process disclosed herein, no complications, such as bleeding or air leak, were observed during the thymolung generation. The engraftment and function of the thymic tissue in the lung, a very delicate organ, is thus a viable and feasible surgical procedure. The present disclosure establishes the engraftment and function of the thymic tissue in this anatomic location (i.e., thymolung) as an alternative to the ThymoKidney Transplantation (TKT) described herein.

### Robotic Auto ThymoKidney Transplantation

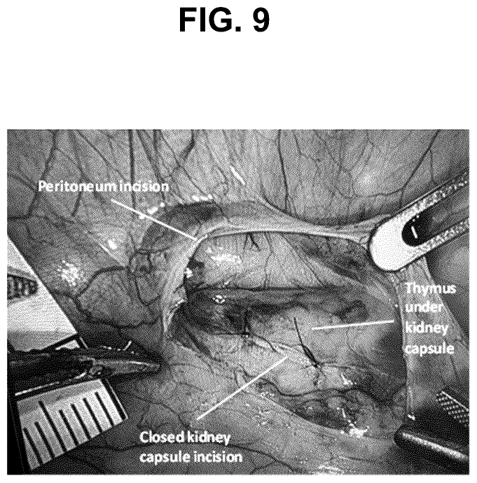

The present disclosure also provides a robotic auto ThymoKidney transplantation. As shown in the examples, the present disclosure illustrates the feasibility of a robotic auto ThymoKidney transplantation using the da Vinci® Xi™ Surgical System or the da Vinci SP® Surgical System (Intuitive Surgical, Sunnyvale, CA, USA) (FIG. 9).

The present disclosure shows for the first time that Thymokidney can be generated at more than one location using a single incision with a da Vinci® Xi™ Surgical System or similar robotic system. For example, in some embodiments, the Thymokidney can be generated at one location, 2 locations, 3 locations, 4 locations, 5 locations, 6 locations, or more locations of the same kidney using a single surgical incision. In some embodiments, the placement of thymus tissue pieces procedure can be repeated at least twice, at least 3 times, at least 4 times, at least five times or more on other locations of the kidney.

The robotic auto ThymoKidney transplantation showed several advantages. Access to either kidneys from the abdominal midline was possible as shown with the da Vinci SP® Surgical System and the SP access port in example 6. Furthermore, different techniques can be applied to incise the kidney capsule and elevate the capsule to create the cavity. For example, scissors, a suture needle, or a syringe needle can be used. Example 6 also shows for the first time that air insufflation helped to create a large sub-capsular cavity for transplanting thymus tissue pieces. Long forceps-style instruments were also functionally able to insert the thymus tissue into the cavity. Lastly, closure of the capsule and peritoneum was possible with sutures. The present disclosure shows that thymokidneys can be generated using robots, such as the Intuitive Surgical' s DaVinci surgical system or any equivalent thereof. It seems reasonable that the new robotic surgery described herein can be established as a routine procedure. The da Vinci® Xi™ Surgical System is a particularly suitable robot for TKT surgeries due to its size (e.g., height) and the wider range of available instruments.

### Donor-Reactive T Cells and Innate Immune Cells Promoted Pig-to-Human Xenograft Rejection

Xenotransplantation of pig organs is a promising solution to the organ shortage; however, rejection remains a major obstacle. Pig-to-decedent human transplantation provides an opportunity to study immune barriers to xenotransplantation experimentally. Donor-reactive T cell dynamics were tracked in a 61-day pig-to-decedent human kidney xenotransplant. Xenogeneic donor-reactive T cell clones (XDRTCCs) identified using high-throughput TCRB CDR3 sequencing expanded markedly in peripheral blood in association with apparent antibody-mediated rejection (AMR). Single-cell RNA and TCR sequencing of leukocytes from the graft showed XDRTCC infiltration and effector transcript expression during AMR. Additionally, γδ and NK cells with cytotoxic effector phenotypes were prominent in the rejecting xenograft. These data suggest that improved suppression of innate immunity and T cell responses might enhance the success of xenotransplantation.

### II. Definitions

Unless defined otherwise, all technical and scientific terms used herein generally have the same meaning as commonly understood by one of ordinary skill in the art to which this technology belongs. As used in this specification and the appended claims, the singular forms “a,” “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “Adverse event” refers to any unfavorable or unintended sign (including an abnormal laboratory finding, for example), symptom, or disease temporarily associated with the use of a medicinal product (e.g., a xenotransplant), whether or not considered related to the medical product.

As used herein, the term “Animal” refers to a mammal. In specific embodiments, the animals are at least six months old. In certain embodiments, the animals are postweaning age. In certain embodiments, the animal survives to reach breeding age. The animals of the present disclosure can be “genetically modified” or “transgenic,” which means that they have a transgene, or other foreign DNA, added or incorporated, or an endogenous gene modified, including, targeted, recombined, interrupted, deleted, disrupted, replaced, suppressed, enhanced, or otherwise altered, to mediate a genotypic or phenotypic effect in at least one cell of the animal and typically into at least one germ line cell of the animal.

In some embodiments, the animal may have the transgene integrated on one allele of its genome (heterozygous transgenic). In other embodiments, the animal may have the transgene on two alleles (homozygous transgenic).

As used herein, the term “macerating” refers to mincing or slicing a tissue into a specified size. In some embodiments, macerating may mean slicing a donor thymic tissue into about 0.1 mm, about 0.2 mm, about 0.3 mm, about 0.4 mm, about 0.5 mm, about 0.6 mm, about 0.7 mm, about 0.8 mm, about 0.9 mm, about 1.2 mm, about 1.3 mm, about 1.4 mm, or about 1.5 mm slice.

As used herein, the term “mammal” can refer to a rodent (i.e., a mouse, a rat, or a guinea pig), a monkey, a cat, a dog, a cow, a horse, a pig, or a human. In some embodiments, the mammal is a human.

As used herein, the term “subject” can refer to any animal, including mammals, such as mice, rats, other rodents, rabbits, dogs, cats, swine, cattle, sheep, horses, or primates, such as humans.

As used herein, the term “tissue” can refer to any type of tissue in human or animals, and can include, but is not limited to, thymic tissue, vascular tissue, skin tissue, hepatic tissue, pancreatic tissue, neural tissue, urogenital tissue, gastrointestinal tissue, skeletal tissue including bone and cartilage, adipose tissue, connective tissue including tendons and ligaments, amniotic tissue, chorionic tissue, dura, pericardia, muscle tissue, glandular tissue, facial tissue, or ophthalmic tissue

As used herein, the term “tolerance” can refer to the inhibition or decrease of a graft recipient's ability to mount an immune response to a donor antigen, which would otherwise occur in response to the introduction of a non self MHC antigen into the recipient. Tolerance can involve humoral, cellular, or both humoral and cellular responses. Tolerance can include any degree of inhibition of a graft recipient's ability to mount an immune response to a donor antigen.

As used herein, the term “transplant tolerance” can be defined as a state of donor-specific unresponsiveness without a need for ongoing pharmacologic immunosuppression. Transplantation tolerance could eliminate many of the adverse events associated with immunosuppressive agents. As such, induction of tolerance may result in improved receipt of a xenograft. In an embodiment, induction of immune tolerance may be identified by a decrease in clinical symptoms of xenograft rejection.

In another embodiment, induction of immune tolerance may ameliorate or prevent the metabolic, inflammatory, and proliferative pathological conditions or diseases associated with xenograft transplantation. In still another embodiment, induction of immune tolerance may ameliorate or decrease or prevent the adverse clinical conditions or diseases associated with the administration of immunosuppressive therapy used to prevent xenograft rejection. In still yet another embodiment, induction of immune tolerance may promote xenograft survival. In a different embodiment, induction of immune tolerance may prevent relapses in patients exhibiting these diseases or conditions.

### III. Tolerance Induction

The present disclosure provides methods of inducing immune tolerance of a xenotransplant organ in a xenotransplant recipient using an improved composite thymo-kidney organ. The methods disclosed herein generally comprise, consist essentially of, or consist of obtaining a thymus from a young donor animal (e.g., a 16-20 week-old pig), macerating the thymus tissue; optionally, culturing the macerated thymus tissue in a culture regimen, injecting the macerated thymus tissue into the sub-renal capsule of the same animal (e.g., autologous) or a first recipient of the same species as the donor animal (e.g., heterologous) using the methods disclosed herein, allowing the macerated thymus tissue to mature into a composite thymo-kidney organ for about 4-12 weeks, and generating a composite thymo-kidney organ (e.g., UThymoKidney). The composite thymo-kidney organ is then collected and transplanted the same day in a subject in need thereof.

In some embodiments of any of the methods disclosed herein, the macerated thymic tissue or thymic tissue is injected into a lung of a donor animal or a first recipient. In some embodiments, the macerated thymic tissue or thymic tissue is injected into an intrapulmonary cavity. In some embodiments, the intrapulmonary cavity can contain 3-5 g of macerated thymic tissue or thymic tissue. In some embodiments, the intrapulmonary cavity can contain about 1 g, about 1.5 g, about 2.5 g, about 3 g, about 3.5 g, about 4 g, about 4.5 g, about 5 g, or more grams of macerated thymic tissue or thymic tissue. In some embodiments, the intrapulmonary cavity can contain about 3.5 g of macerated thymic tissue or thymic tissue. In some embodiments, the macerated thymic tissue or thymic tissue comprises about one-inch (1″) long and about quarter-inch (¼″) diameter pieces.

In some embodiments, the macerated thymic tissue or thymic tissue is injected into a lung parenchyma or pleura. In some embodiments, the macerated thymic tissue or thymic tissue is injected into a lung intra-parenchymal cavity. In some embodiments, the macerated thymic tissue or thymic tissue is injected into a lung sub-pleural cavity. In some embodiments, a thymolung is generated.

### A. Macerated Thymus Tissue

One aspect of the present disclosure provides a method of inducing a transplant tolerance, the method comprising, consisting of, or consisting essentially of, macerating a thymic tissue obtained from a donor animal; injecting the macerated thymic tissue into a sub-renal capsule of the donor animal; maturing the injected macerated thymic tissue into a composite renal subcapsular thymic lymphoid tissue; and generating a composite thymo-kidney organ. In some embodiments, the donor animal is thymectomized prior to the injection of the macerated thymic tissue into a sub-renal capsule of the donor animal. In some embodiments, the donor animal is partially thymectomized using a blunt and sharp dissection.

In this embodiment, macerating comprises, consists essentially of, or consists of slicing the thymus tissue into about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.2 mm, about 1.3 mm, about 1.4 mm, or about 1.5 mm pieces. In some embodiments, the thymus tissue is sliced into about 0.4 mm pieces.

In some embodiments, the macerated thymic tissue is injected subcapsularly into the kidney of the donor animal. In some embodiments, the subcapsular injection is performed using a series of steps. In particular, the method can comprise: (a) placing the donor animal in a lateral position; (b) introducing a horizontal or vertical incision extending from the last rib into the paralumbar fossa and over the kidney; mobilizing the caudal pole and lateral aspect of the kidney; (d) exposing the kidney within a Gerota's fascia; (e) introducing a small incision in the kidney capsule; (f) separating the renal capsule from the renal cortex to generate a pocket; (g) injecting the macerated thymic tissue into the pocket; (h) closing the kidney capsule and (i) closing the horizontal or the lateral incision.

In some embodiments, the horizontal or vertical incision is about 5 cm to about 8 cm. In some embodiments, the kidney capsule incision is closed with a Prolene 6-0 suture. In some embodiments, the kidney capsule incision is cauterized.

The renal subcapsular space is accessible and nutrition-rich. As such, it is used for various engraftment needs. However, the kidney capsule is fragile. Attempts to place large volumes of cells or tissues often fail. Renal subcapsular transplantation is associated with a high risk of cell loss, tissue dislocation, or injury to the capsule. Indeed, during transplantation, sharp needles can tear the capsule or injure the renal cortex and/or renal parenchyma. It has been shown that injecting the thymic tissue using a conventional angiocatheter resulted in poor results.

Accordingly, the present disclosure provides an improved method of engrafting a tissue under the kidney capsule. The placement of the macerated thymic tissue is important for generating a functional composite ThymoKidney that would produce competent T cells. In some embodiments, the injected macerated thymic tissue covers one surface of the caudal renal pole. Specifically, the amount of macerated thymic tissue should be sufficient to cover the surface of the caudal renal pole.

In some embodiments, the amount of injected macerated thymic tissue is less than about ⅕ (20%) of the kidney. In some embodiments, the amount of macerated thymic tissue is less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, or less than about 20% of the kidney. In some embodiments, the amount of injected macerated thymic tissue is less than about 40% of the kidney.

In some embodiments, the subcapsular injection can be performed using a robot, a silicone catheter, a blunted needle, or a modified pipette. In some embodiments, the subcapsular injection can be performed using a robot (e.g., da Vinci® Xi™ Surgical System or the da Vinci SP® Surgical System). In some embodiments, the subcapsular injection can be performed using a silicone catheter. In some embodiments, the subcapsular injection can be performed using a blunted needle. In some embodiments, the subcapsular injection can be performed using a modified pipette. The pipette can be modified to have a refined tip. In some embodiments, the silicone catheter, the blunted needle, or the modified pipette can be used to gently separate the capsule from the renal cortex or parenchyma. In some embodiments of the methods described herein, the macerated thymic tissue is injected subcapsularly toward the cranial pole of the kidney capsule.

In some embodiments, the macerated thymic tissue can be suspended in an extracellular matrix composition or a biomaterial prior to injecting the macerated thymic tissue into the sub-renal capsule. In that embodiment, the method comprises waiting for the matrix to solidify prior to closing the kidney capsule. In some embodiment, the kidney capsule is closed with for example a Prolene 6-0 suture. In some embodiments, the kidney capsule incision is cauterized.

In some embodiments, the extracellular matrix composition comprises a collagen matrix, a laminin matrix, a fibronectin matrix, a collagen/laminin matrix, or Matrigel™. In some embodiments, the extracellular matrix composition comprises Matrigel™.

### B. Macerated Thymus Tissue With Culture

Another aspect of the present disclosure comprises a method of inducing a transplant tolerance, the method comprising, consisting essentially of, or consisting of: (a) macerating a thymic tissue obtained from a donor animal; (b) culturing the macerated thymic tissue in a culture regimen for about 1 day to about 30 days; injecting the macerated thymic tissue into a sub-renal capsule or a quadriceps muscle of the donor animal or a first recipient; (c) maturing the injected macerated thymic tissue into a composite renal subcapsular thymic lymphoid tissue or a composite musculo-thymic lymphoid tissue; and (d) generating a composite thymo-kidney organ or a thymo-muscle organ. In some embodiments, the donor animal or the first recipient is thymectomized prior to the injection.

In some embodiments, culturing the macerated thymic tissue can induce or promote immune competence. In some embodiments, culturing the macerated thymic tissue can increase the number of mature functional T cells in the transplanted subject when compared to uncultured macerated thymic tissue. In some embodiments, culturing the macerated thymic tissue can increase the number of subject-mature functional T cells. In some embodiments, culturing the macerated thymic tissue can provide mature functional donor-T cells, which have matured in the recipient.

In some embodiments, culturing the macerated thymic tissue can improve the immune-responsiveness of the subject. For example, culturing can improve the responsiveness of a T cell of the recipient.

In some embodiments, the macerated thymic tissue is injected into a quadriceps muscle of the donor animal. In some embodiments, the macerated thymic tissue is injected into a quadriceps muscle of a recipient animal.

In some embodiments, the macerated thymic tissue is injected int a sub-renal capsule of the donor animal. In some embodiments, the macerated thymic tissue is injected int a sub-renal capsule of a first recipient animal. In some embodiments, the macerated thymic tissue is injected subcapsularly into the kidney of the donor animal or the first recipient.

In some embodiments, the macerated thymic tissue is injected subcapsularly into the kidney of the donor animal. In some embodiments, the subcapsular injection is performed using a series of steps. In particular, the method can comprise: (a) placing the donor animal in a lateral position; (b) introducing a horizontal or a vertical incision extending from the last rib into the paralumbar fossa and over the kidney; mobilizing the caudal pole and lateral aspect of the kidney; (d) exposing the kidney within a Gerota's fascia; (e) introducing a small incision in the kidney capsule; (f) separating the renal capsule from the renal cortex to generate a pocket; (g) injecting the macerated thymic tissue into the pocket; (h) closing the kidney capsule and (i) closing the horizontal or the lateral incision.

In some embodiments, the horizontal or the vertical incision is about 5 cm to about 8 cm. In some embodiments, the kidney capsule incision is closed with a Prolene 6-0 suture. In some embodiments, the kidney capsule incision is cauterized.

Accordingly, the present disclosure provides an improved method of engrafting a tissue under the kidney capsule. The placement of the macerated thymic tissue is important for generating a functional composite ThymoKidney that would produce competent T cells. In some embodiments, the injected macerated thymic tissue covers one surface of the caudal renal pole. Specifically, the amount of macerated thymic tissue should be sufficient to cover the surface of the caudal renal pole.

In some embodiments, the amount of macerated thymic tissue is less than about ⅕ (20%) of the kidney. In some embodiments, the amount of macerated thymic tissue is less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, or less than about 20% of the kidney. In some embodiments, the amount of macerated thymic tissue is less than about 40% of the kidney.

In some embodiments, the subcapsular injection can be performed using a robot, a silicone catheter, a blunted needle, or a modified pipette. In some embodiments, the subcapsular injection can be performed using a silicone catheter. In some embodiments, the subcapsular injection can be performed using a blunted needle. In some embodiments, the subcapsular injection can be performed using a modified pipette. The pipette can be modified to have a refined tip. In some embodiments, the silicone catheter, the blunted needle, or the modified pipette can be used to gently separate the capsule from the renal cortex or parenchyma. In some embodiments of the methods described herein, the macerated thymic tissue is injected subcapsularly toward the cranial pole of the kidney capsule.

In some embodiments, the macerated thymic tissue is cultured for about 1 to about 7 days, about 5 to about 7 days, about 5 to about 12 days, about 5 to about 9 days, about 10 to about 20 days, about 5 to about 12 days, about 5 to about 15 days, or about 10 to about 20 days. In some embodiments, the macerated thymic tissue is cultured for at least about 15 days. In some embodiments, the macerated thymic tissue is cultured for at least about 12 days.

The culturing is preferably done at 37 °C in a 5% CO₂ incubator. In some embodiments, the culturing comprises placing a macerated thymus tissue on cellulose (e.g., Millipore® cellulose) or equivalent filters in tissue culture dishes.

In some embodiments, culturing comprises changing a culture medium daily. In some embodiments, a culture regimen comprises the University of Wisconsin (UW) solution. In some embodiments, a culture regimen comprises a thymus organ medium. In some embodiments, the thymus organ medium can comprise about 86.5% HAMS F12, about 25 mM Hepes; about 2 mM L-Glutamine; about 10% Fetal Bovine Serum; and about 1× Pen-strep.

In some embodiments, culturing comprises contacting the macerated thymus tissue with an agent that depletes the macerated thymus tissue of thymocytes, while keeping the thymic stromal cells alive. In some embodiments, the agent is selected from the group consisting of 2-deoxyguanosine (2DG), cyclosporine A, hydrocortisone, notch inhibitors, ABT-737, and 2D glucose, or a combination of 2 or more thereof.

In some embodiments, macerating comprises contacting the thymic tissue with 2-DG. In that embodiment, culturing comprises contacting the thymic tissue with about 1 nM to about 500 nM 2-DG, about 25 nM to about 250 nM 2-DG, about 50 nM to about 150 nM 2-DG, about 50 nM to about 100 nM 2-DG, about 100 nM to about 300 nM 2-DG, or about 250 nM to about 500 nM 2-DG.

In some embodiments, the thymic tissue is cultured with 2-DG for about 2 hours to about 24 hours. In some embodiments, the thymic tissue is cultured with 2-DG for about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 16 hours, about 17 hours, about 18 hours, about 19 hours, about 20 hours, about 21 hours, about 22 hours, about 23 hours, or about 24 hours. In some embodiments, the thymic tissue is cultured with 2-DG for about 12 hours.

In some embodiments, culturing comprises contacting the thymic tissue with about 100 nM 2-DG for about 10 hours. In some embodiments, culturing comprises contacting the thymic tissue with about 100 nM 2-DG for about 12 hours. In some embodiments, culturing comprises contacting the thymic tissue with about 100 nM 2-DG for about 15 hours. In some embodiments, culturing with 2-DG depletes the macerated thymus tissue of thymocytes, while keeping the thymic stromal cells alive.

In some embodiments, the culture regimen depletes the thymus tissue of donor thymocytes; or maintains thymic stromal components. In some embodiments, culturing produces T-cell depleted donor macerated thymic tissue. In some embodiments, culturing enriches thymic epithelial cells and other stromal cells in the macerated thymic tissue.

Thymic stromal components can comprise Thymic epithelial cells (TECs). Thymic epithelial cells are further divided into cortical and medullary TECs based on their localization within the thymus. Cortical TECs may be involved in positive selection of T cells and medullary TECs may be involved in negative selection of T cells. Cortical TECs are EpCAM⁺Ly-51⁺CD45⁻ and generally express keratin 8 (KRT8). Medullary TECs are EpCAM⁺UEA-1⁺CD45⁻, and generally express keratin 5 (KRT5). Medullary TECs are also characterized by a reactivity with Ulex europaeus agglutinin I (UEA-1).

In some embodiments, culturing comprises suspending the thymic tissue in an extracellular matrix composition or a biomaterial prior to injecting the macerated thymic tissue into the sub-renal capsule or the quadriceps muscle of the donor animal or the first recipient.

In some embodiments, the extracellular matrix composition comprises a collagen matrix, a laminin matrix, a fibronectin matrix, a collagen/laminin matrix, or Matrigel™. In some embodiments, the extracellular matrix composition comprises Matrigel™.

In some embodiments, the biomaterial may be a sol-gel, a hydrogel laden with proteins, a Matrigel™, an artificially constructed scaffold with cells, and combinations thereof. Non-limiting examples of the biomaterials may also include, polyethylene-imine and dextran sulfate, poly(vinylsiloxane)ecopolymerepolyethyleneimine, phosphorylcholine, poly(ethylene glycol), poly(lactic-glycolic acid), poly(lactic acid), polyhydroxyvalerte and copolymers, polyhydroxybutyrate and copolymers, polydiaxanone, polyanhydrides, poly(amino acids), poly(orthoesters), polyesters, collagen, gelatin, cellulose polymers, chitosans, alginates, fibronectin, extracellular matrix proteins, vinculin, agar, agarose, hyaluronic acid, and combinations thereof.

The methods disclosed herein improve the tolerance of a xenograft from a donor species in a recipient of a different species by partially or fully depleting the donor thymus of all or most thymocytes. In some embodiments, the methods disclosed herein deplete the donor thymus of thymocytes (e.g., CD8⁺ memory T cells). In some embodiments, the methods described herein enhance the activity of thymic stromal components. In some embodiments, the methods described herein improve the perseverance and activity of Thymic epithelial cells (TECs) in the composite thymo-kidney organ.

### C. Macerated Thymus Tissue With Thymic Epithelial Cells Culture

In some embodiments of the methods described herein, culturing further comprises co-culturing the macerated thymic tissue with isolated thymic epithelial cells obtained from a subject to be transplanted. In some embodiments, the thymic tissue and the isolated thymic epithelial cells from the subject are injected into the sub-renal capsule or the quadriceps muscle of the donor animal or the first recipient. In some embodiments, the isolated thymic epithelial cells (TECs) are engineered isolated thymic epithelial cells.

Thymopoiesis is the process by which thymocytes are turned into mature T cells according to either negative or positive selection. Thymopoiesis releases a repertoire of about millions of unique T cell receptors per individual. The thymus is responsible for the generation and maturation of T cells. This role is generally attributed to Thymic epithelial cells (TECs), which account for the majority of thymic stromal components. Thymic epithelial cells are further divided into cortical and medullary TECs based on their localization within the thymus. Cortical TECs may be involved in positive selection of T cells and medullary TECs may be involved in negative selection of T cells.

Accordingly, co-culturing the macerated thymic tissue with isolated TECs from the subject and/or subcapsularly co-injecting the macerated thymic tissue and isolated TECs can generate a composite thymus-kidney organ with enhanced activity.

### D. Thymus Maturation

In some embodiments of any of the methods of inducing a transplant tolerance disclosed herein, the subcapsular injected macerated thymic tissue is allowed to mature for about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, or about 20 weeks. In some embodiments, the subcapsular injected macerated thymic tissue is allowed to mature for about 4 weeks to about 10 weeks, about 4 weeks to about 8 weeks, about 5 weeks to about 10 weeks, or about 6 weeks to about 8 weeks.

### E. Procurement and Xenotransplantation

In some embodiments of any of the methods of inducing a transplant tolerance disclosed herein, the method further comprises transplanting the composite thymus-kidney organ in a subject in need of a kidney transplant.

In some embodiments, the composite thymus-kidney organ induces thymopoiesis in the subject within about 1 month, about 2 months, about 3 months, about 4 months, about 5 months, about 6 months, about 7 months, about 8 months, about 9 months, about 10 months, about 11 months, or about 12 months.

### IV. Genetically Modified Pigs

One aspect of the present disclosure provides genetically modified animals (e.g., pigs; donor) expressing at least 10 genetic modifications. The generation of the genetically modified pig for used in the method disclosed herein is described below and in WO 2023/044100, which is incorporated herein by reference in its entirety.

To further mitigate the risk of T-cell mediated immune activation following xenotransplantation, the donor animal can be a genetically modified animal (e.g., a pig). Accordingly, in some embodiments, the donor or the first recipient is a genetically modified pig. In some embodiments, the donor or the first recipient is a transgenic pig that: lacks expression of a functional gene selected from the group consisting of an alpha 1,3 galactosyltransferase, a growth hormone receptor (GHR), a β-1,4-N-acetyl-galactosaminyltransferase 2 (β4GalNT2), and cytidine monophosphate-N-acetylneuraminic acid hydroxylase (CMAH), or a combination thereof; and expresses at least six exogenous transgenes selected from an anticoagulant transgene, a cytoprotective transgene, an immunosuppressant transgene, or a complement inhibitor transgene, or a combination thereof.

In some embodiments, the donor or the first recipient is a transgenic pig that lacks expression of a functional alpha 1,3 galactosyltransferase, a growth hormone receptor (GHR), a β-1,4-N-acetyl-galactosaminyltransferase 2 (β4GalNT2), and cytidine monophosphate-N-acetylneuraminic acid hydroxylase (CMAH); and expresses at least six exogenous transgenes selected from an anticoagulant transgene, a cytoprotective transgene, an immunosuppressant transgene, and a complement inhibitor transgene.

In some embodiments, the cytoprotective transgene is selected from the group consisting of heme oxygenase 1 (HO-1), A20, FAT-1, and soluble tumor necrosis factor-alpha (TNF-alpha), or a combination thereof. In some embodiments, the immunosuppressant transgene is selected from the group consisting of Cytotoxic T-Lymphocyte-Associated Protein 4 (CTLA4), cluster of differentiation 47 (CD47), and Class II transactivator-DN (CIITA-DN) or a combination thereof. In some embodiments, the anticoagulant transgene is selected from the group consisting of endothelial protein C receptor (EPCR), thrombomodulin, CD39, hirudin, and Tissue factor pathway inhibitor (TFPI), or a combination thereof. In some embodiments, the complement inhibitor is selected from the group consisting of CD46, DAF (CD55), CD59, and CR1, or a combination thereof. In some embodiments of the method described herein, the donor or first recipient comprises an exogenous CD47 transgene.

In some embodiments, the donor animal is about 1 month, about 2 months, about 3 months, about 4 months, about 5 months, about 6 months, about 7 months, about 8 months, about 9 months, about 10 months, about 11 months, about 12 months, about 24 months, about 36 months, about 48 months, about 60 months, about 72 months, about 84 months, about 96 months, about 108 months, or about 120 months.

In some embodiments, the donor animal is about 4 month old. In some embodiments, the donor animal is about 5-month old. In some embodiments, the donor animal is about 16 weeks to about 20 weeks. In some embodiments, the donor animal is about 16 weeks, about 17 weeks, about 18 weeks, about 19 weeks, or about 20 weeks.

In some embodiments, organs, organ fragments, tissues or cells derived from the genetically modified pig produce low to no levels of one or more of the following: hyperacute rejection (HAR), acute humoral rejection (AHXR/DXR) and/or acute cellular xenograft rejection (ACXR) following xenotransplantation.

In one embodiment, organs, organ fragments, tissues or cells derived from the genetically modified pig produce low to no levels of HAR and AHXR following xenotransplantation. In another embodiment, organs, organ fragments, tissues or cells derived from the genetically modified pig produce low to no levels of HAR, AHXR and ACXR following xenotransplantation. In some

In some embodiments, the genetically modified pig lacks any expression of functional alpha 1,3 galactosyltransferase (alpha Gal) and incorporates at least several additional genetic modifications.

In some embodiments, the genetically modified pig lacks any expression of functional alpha 1,3 galactosyltransferase (alpha Gal) and incorporates and expresses at least six transgenes, under control of at least two promoters, at a single locus. In some embodiments, one promoter controls the expression of at least two transgenes. In alternative embodiments, one promoter controls the expression of more than one transgene, e.g., one promoter controls the expression of two transgenes.

In some embodiments, the six or more transgenes are co-integrated, co-expressed and co-segregated during breeding. The single locus may vary. In some embodiments, the single locus is a native or modified native locus. The modified native locus may be modified by any suitable technique, including, but not limited to, CRISPR-induced insertion or deletion (indel), introduction of a selectable marker gene (e.g., neo) or introduction of a large genomic insert (e.g., a landing pad) intended to facilitate incorporation of one or more transgenes.

In some embodiments, the single locus is a native or modified GGTA1 locus. The GGTA1 locus is inactivated by incorporation and expression of the at least six transgenes, for example by homologous recombination, application of gene editing or recombinase technology. The single locus may be, for example, AAVS1, GHR, ROSA26, CMAH, or ß4GalNT2. In some embodiments, the transgenic animal may have one or more additional genetic modifications and/or the expression of one or more additional porcine genes may be modified by a mechanism other than genetic modification.

In some embodiments, the genetically modified pig lacks any expression of functional alpha 1,3 galactosyltransferase (alpha Gal) and incorporates and expresses at least three, at least four, at least five, at least six, at least seven, at least eight, at least nine, or at least ten transgenes or more at a single locus. In some embodiments, expression of the at least three, at least four, at least five, at least six, at least seven, at least eight, at least nine, or at least ten transgenes or more is controlled by at least two, at least three, at least four, at least five, at least six, at least seven, at least eight, at least nine, at least ten promoters or more. In some embodiments, the promoter is dedicated to the transgene, i.e., one promoter controls expression of one transgene, while in alternative embodiments, one promoter controls expressions of more than one transgene, e.g., one promoter controls expression of two transgenes.

In some embodiments, the two or more additional transgenes are co-integrated, co-expressed and co-segregated during breeding. The single locus may vary. In certain embodiments, the single locus is a native or modified native locus. The modified native locus may be modified by any suitable technique, including, but not limited to, CRISPR-induced insertion or deletion (indel), introduction of a selectable marker gene (e.g., Neo) or introduction of a large genomic insert (e.g., a landing pad) intended to facilitate incorporation of one or more transgenes. In a particular embodiment, the single locus is a native or modified GGTA1 locus.

### EXAMPLES

The present technology is further illustrated by the following Examples, which should not be construed as limiting in any way. The examples herein are provided to illustrate the advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Generation of 6 Gene Vectors

Vector constructions. Multiple bi-cistronic units were synthesized consisting of two (2) transgenes linked by 2A peptide sequences that share a single promoter. Two forms of 2A sequences, P2A (66bp) and T2A (55bp) were utilized to link large number of two-transgene units to allow co-expression of both genes from one promoter. Promoters were either the constitutive CAG promoter (CMV enhancer, chicken actin promoter, rabbit b-globin intron1), the endothelial-specific porcine TBM promoter (pTBMpr), the endothelial-specific porcine ICAM-2 promoter or a combination of the Tie2 endothelial-specific enhancer with the CAG promoter. Pairs of human transgenes were constructed (connected by the 2A sequence) including thrombomodulin (TBM), EPCR, CD59, CD47, HO1, CD46 and CD55 (DAF).

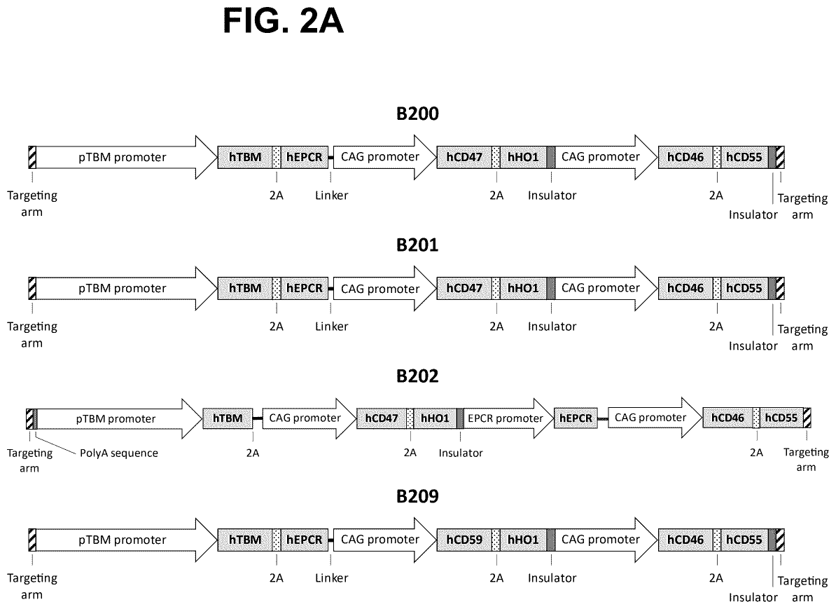

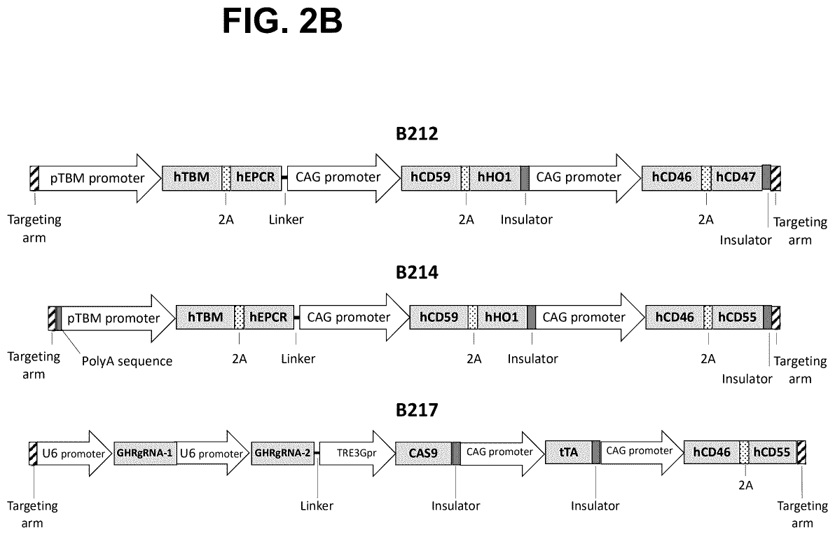

Exemplary embodiments of the vectors of the present invention are shown in FIGS. 2A-B, 3A-2G, and 4.

FIGS. 2A-B show schematics of multigene vectors used to engineer the genetically modified pig. In particular, FIGS. 2A-B show schematic representations of the B200, B201, B202, B209, B212, B214, and B217 multicistronic vectors. B200 vector is a multicistronic vector (MCV) comprising three bi-cistron units (pTBMpr [hTBM-2A-hEPCR]/CAGpr [hCD47-2A-hHO1]/CAGpr [hCD46-2A-hDAF]) flanked by targeting arms for homology directed repair (HDR) at the CMAH gene locus. B201 vector is a MCV comprising three bi-cistron units (PolyA/pTBMpr [hTBM-2A-hEPCR]/CAGpr [hCD47-2A-hHO1]/CAGpr [hCD46-2A-hDAF]) flanked by targeting arms for HDR at GGTA1/Neo gene locus. B202 vector is a MCV comprising two mono-cistron units (PolyA/pTBMpr [hTBM]; pEPCRpr [hEPCR]) and two bi-cistron units, arranged in the following order: (PolyA/pTBMpr [hTBM]; CAGpr [hCD47-2A-hHO1]; pEPCRpr [hEPCR]/CAGpr [hCD46P-2A-hDAF] and flanked by targeting arms for HDR at GGTA1/Neo locus. B209 vector is a MCV comprising three bi-cistron units (pTBMpr [hTBM-2A-hEPCR]/ CAGpr [hCD47-2A-hHO1]/ CAGpr [hCD46-2A-hDAF], flanked by targeting arms for HDR at CMAH locus. B212 vector is a MCV comprising three bi-cistron units (pTBMpr [hTBM-2A-hEPCR]/ CAGpr [hCD59-2A-hHO1]/ CAGpr [hCD46-2A-hCD47]), flanked by targeting arms for HDR at GGTA1/Neo locus. B214 vector is a MCV comprising three bi-cistron units (pTBMpr [hTBM-2A-hEPCR]/ CAGpr [hCD59-2A-hHO1]/ CAGpr [hCD46-2A-hCD55]), flanked by targeting arms for HDR at GGTA1/Neo locus. B217 vector is a MCV comprising five expression units (U6promoter[GHRgRNA-1]/ U6promoter[GHRgRNA-2]/, TRE3G[CAS9]/ CAGpr [tTA]/ CAGpr [hCD46-2A-hCD55]), flanked by targeting arms for HDR at GGTA1/Neo locus.

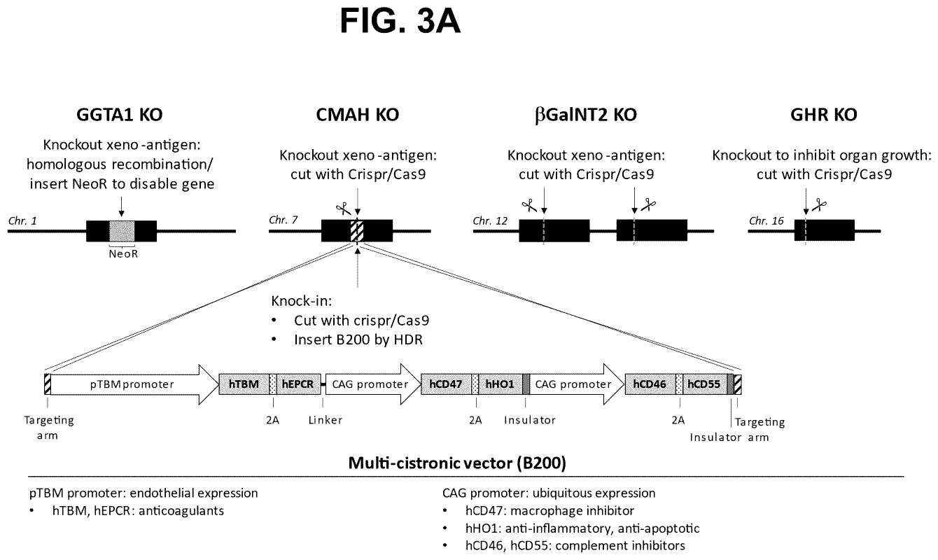

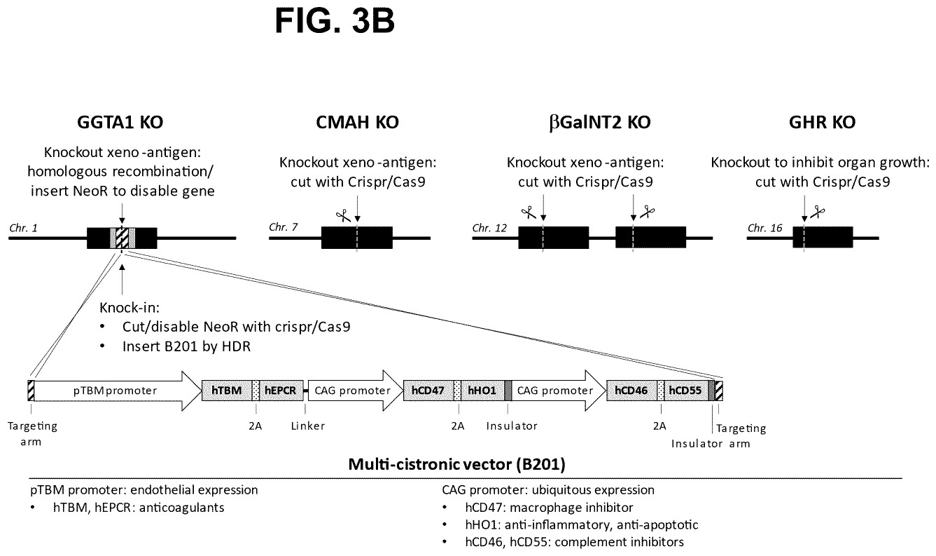

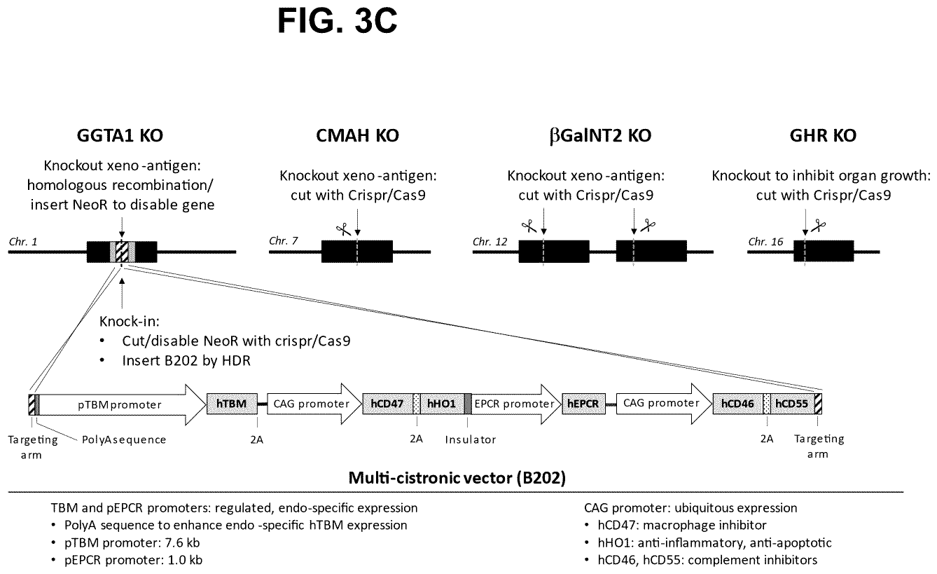

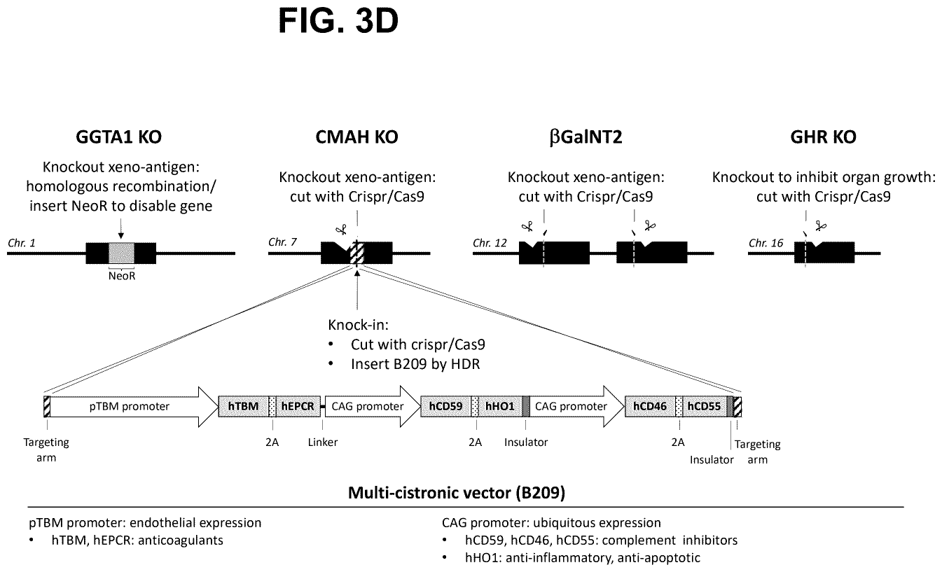

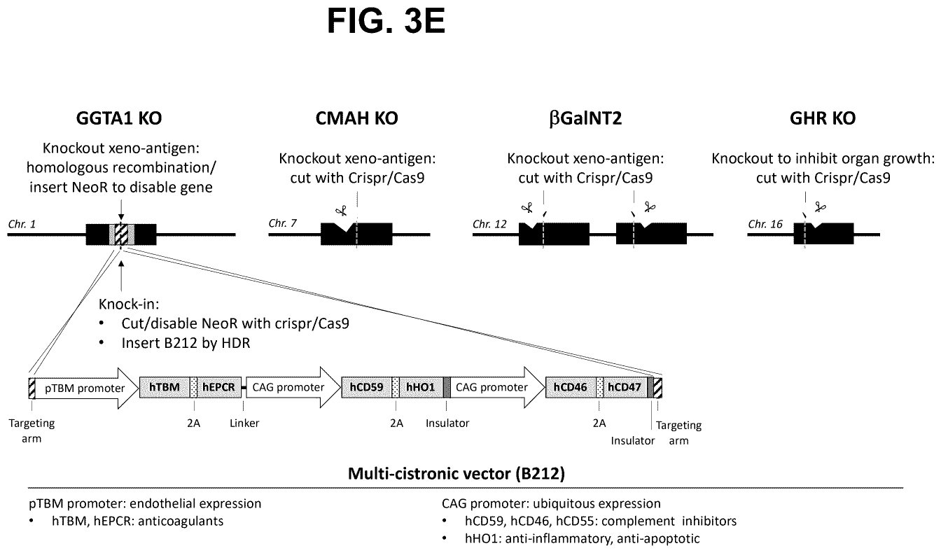

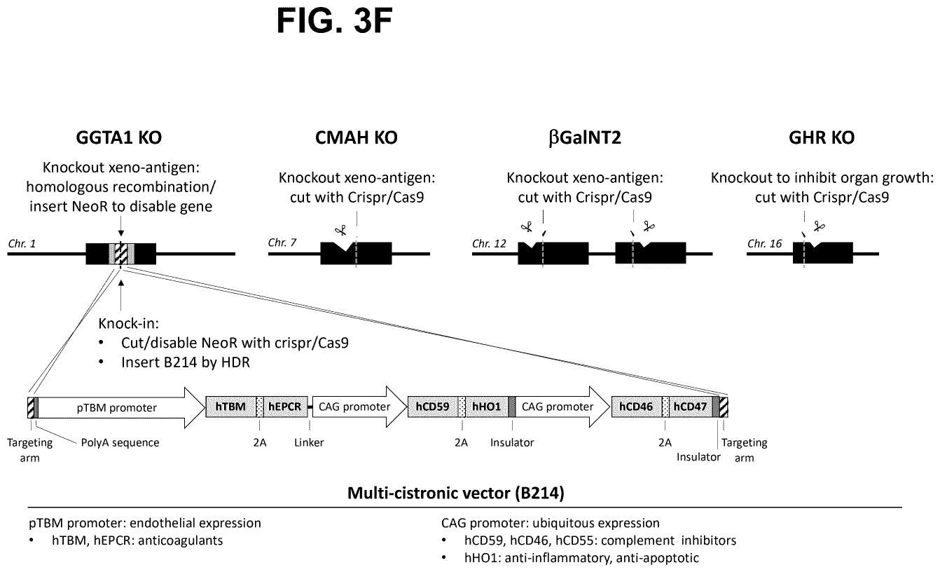

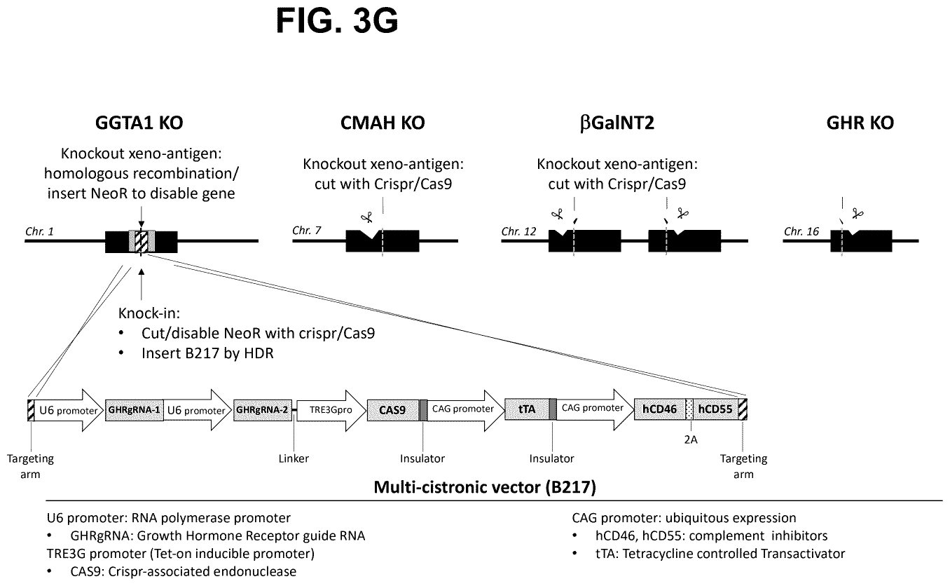

FIGS. 3A-G show schematic representations of the targeted integration of multigene vectors comprising six human transgenes (6-gene vectors) into exemplary loci and embodiments of functional knockouts of at least 4 porcine genes (GGTA1, CMAH, B4GalNT2, and GHR) for producing the genetically modified pig.

FIG. 3A illustrates the insertion of the B200 vector into the CMAH locus. FIG. 3B. illustrates the insertion of the B201 vector into the GGTA1/Neo locus. FIG. 3C. illustrates the insertion of the B202 vector into the GGTA1/Neo locus. FIG. 3D. illustrates the insertion of B209 vector into the CMAH. FIG. 3E. illustrates the insertion of B212 into the GGTA1/Neo locus. FIG. 3F illustrates the insertion of B214 into the GGTA1/Neo locus. FIG. 3G illustrates the insertion of B217 into the GGTA1/Neo locus.

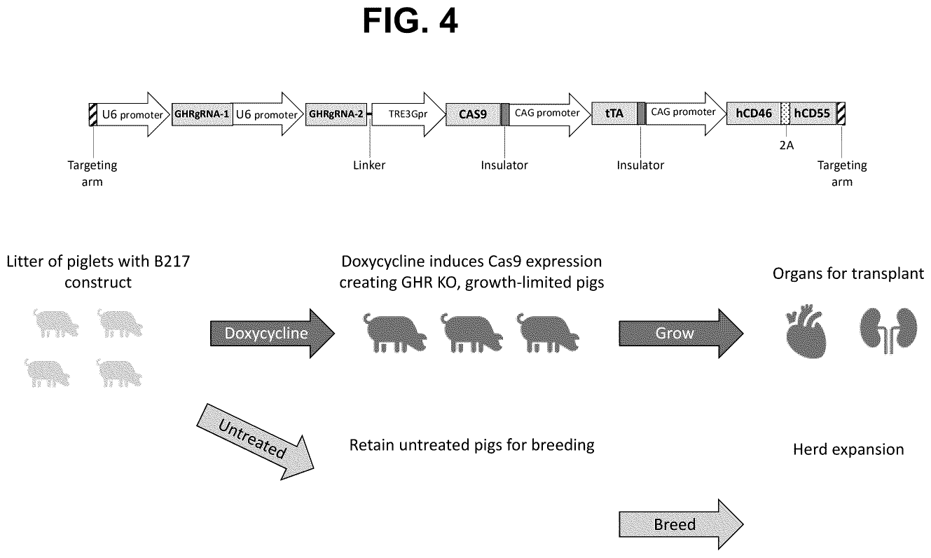

FIG. 4 shows a schematic representation of the production of a multitransgenic animal comprising at least 10 modifications (i.e., 10GE pigs) using the B217 vector.

### Example 2: Generation of a Multitransgenic Animal Comprising at Least 6 Transgenes

Somatic cell nuclear transfer. Live pigs were generated from genetically modified fibroblasts by SCNT, according to the methods described in detail by Giraldo et al. Methods Mol Biol. 885:105-23 (2012).

Screening piglets for genotype. Genotypic characterization of transgenic animals was performed by targeting and transgene PCR analysis, digital copy number PCR analysis, and genomic sequencing analysis as described for above for cell colonies, using DNA extracted from tail pig biopsies. In addition, Southern Blots were done to confirm targeting of the intact vector and the absence of random integrations. Collectively these methods identify and confirm that the targeting vector was integrated at the targeted allele(s), that the vector is intact and that the construct has not otherwise integrated randomly into the genome.

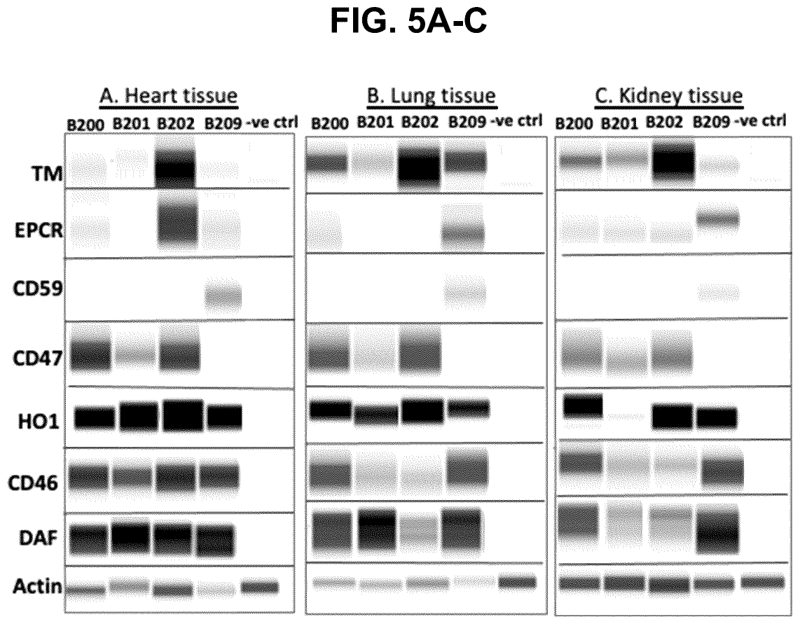

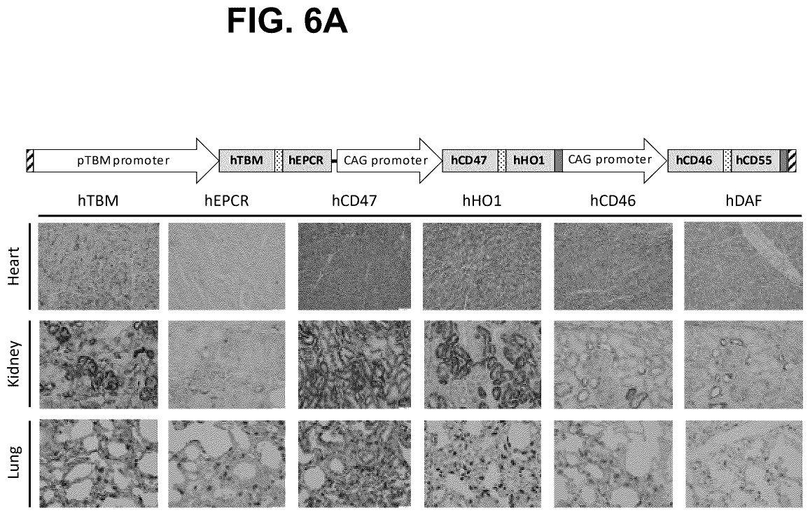

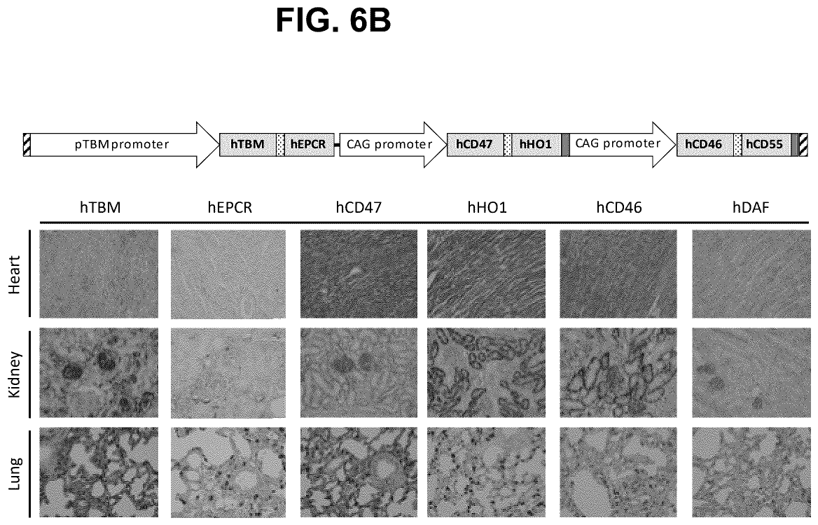

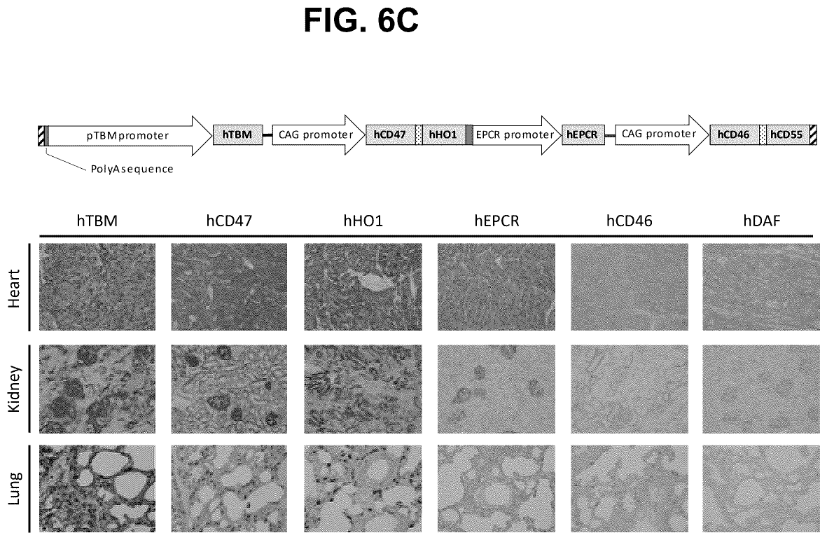

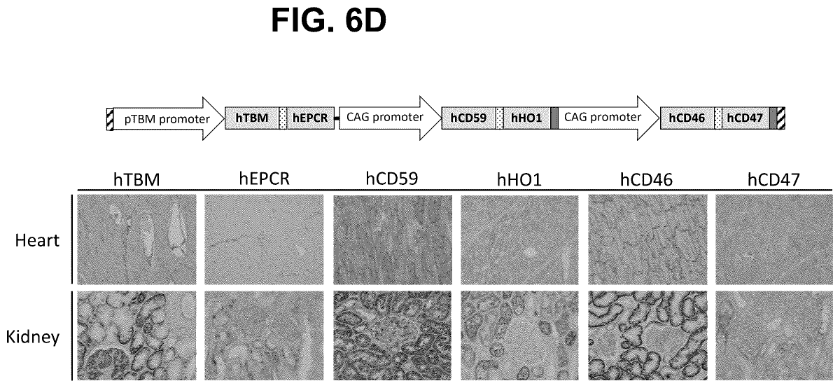

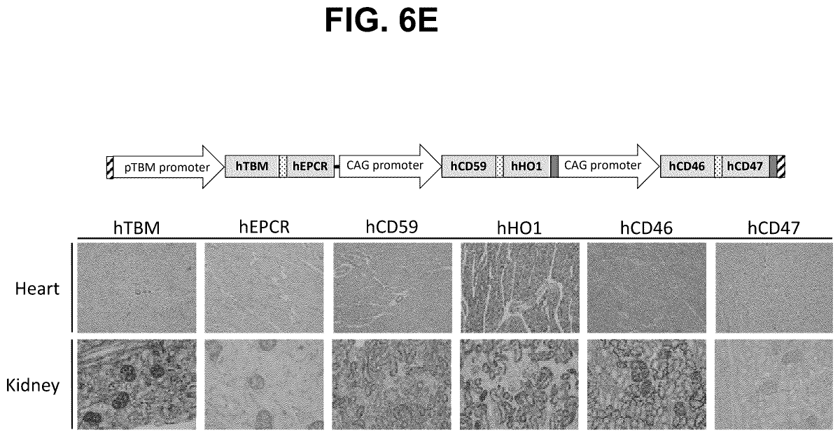

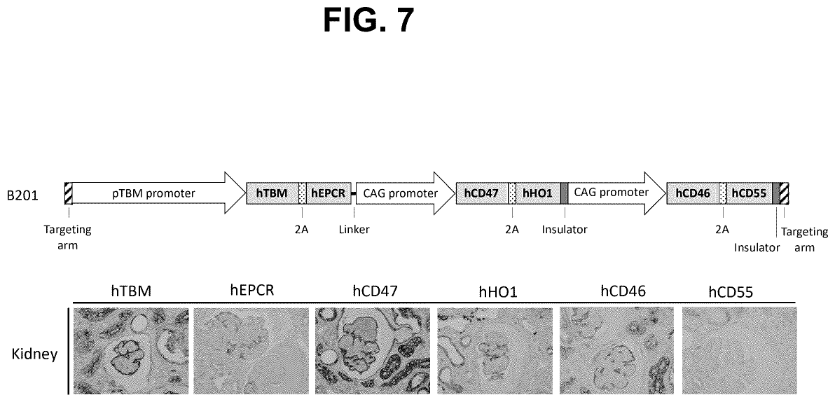

Expression of human transgenes in porcine tissues. Expression of all human transgenes from each vector was confirmed in heart, lung, and kidney samples by western blot (FIGS. 5A-C), and immunohistochemical staining (FIGS. 6A-6E, and 7).

5A-C show a western blot analysis of various tissues from a genetically modified animal expressing a multitransgenic vector and demonstrating the consistent protein expression of the transgenes encoded by the B200, B201, B202, and B209 in heart (FIG. 5A), lung FIG. 5B) and kidney (FIG. 5C). Transgenic proteins from transgenic tissue samples had expected molecular weight by western blot and showed substantially similar expression levels across tissues.

Each of FIGS. 6A-6E, and 7 shows tissue sections stained for transgene expression by immunohistochemistry. Fresh tissue samples were fixed in paraformaldehyde, embedded in paraffin blocks, cut on a microtome, and affixed to glass slides. Sections were probed with primary antibodies human-specific to each protein expressed by the transgenes, then with appropriate second antibodies conjugated to horseradish peroxidase. Antibody-bound proteins were visualized with diaminobenzidine tetrahydrochloride (DAB) as indicated by the brown staining. Positive controls (tissues with previously verified transgene expression) and negative controls (processed without primary antibody) were included in each staining run, but these were not included in the figures.

FIGS. 6A-E show representative images of immunohistochemical stainings of porcine lung, heart, and kidney tissues expressing human transgenes encoded by vectors B200 (FIG. 6A), B201 (FIG. 6B) and B202 (FIG. 6C), and of porcine heart and kidney tissues expressing human transgenes encoded by vectors B209 (FIG. 6D) and B212 (FIG. 6E). Transgenic proteins from transgenic tissue samples showed substantially similar expression levels across tissues.

### Example 3: B201 Supported Long-Term Kidney Survival After Transplantation Into Baboons

Porcine kidneys with B201, GHRKO and two xenoantigen knockouts (GGTA1, β4GalNT 2) were transplanted into baboons and supported life for up to 120 days. Some signs of delayed rejection might have been noted at Day 120 in one recipient. Immunohistochemistry indicated that all six transgenes in B201 were expressed in post-mortem kidney tissue obtained at Day 120 post-transplant (FIG. 7). Another transplanted kidney was functioning well for 66 days. The kidneys in neither recipient underwent hyperacute rejection (HAR).

### Example 4: Methods for Generating a Composite Thymic Kidney Organoids for Inducing Immune Tolerance

### Thymectomy

Pig preparation. The process of generating a composite thymic kidney organoids began once the genetically modified donor animal reached the age of about 16 weeks to about 20 weeks. All personnel involved were required to wear a gown, shoe covers, gloves, bouffant caps, and face masks to work with animals. The animal's food was withheld for about 24 hours prior to surgery, and water was withheld for about 12 hours.

The donor animal was weighed and then pre-anesthetized in the prep room with a combination of Telazol (3 mg/kg IM) and Xylazine (2 mg/kg IM) or BAM® solution at up to 2.0 ml/100 lbs IV or IM. The donor received oxygen by mask (1-4 l/min). If for any reason, the spontaneous breathing was insufficient (low SatO₂ in the pulse oximetry) or if desired by the anesthesia technician, the donor was intubated and ventilated mechanically (100% O₂, tidal volume 7-10 ml/kg BW, respiration rate of about 10-15/min, settings adjusted according to ECO₂ and SatO₂). The donor was then monitored. Monitoring included pulse oximetry (SatO₂, heart rate), ECO₂, temperature and non-invasive blood pressure.

The entire pig was washed. The surgical areas was clipped and cleaned using Nolvasan Scrub and 70% isopropyl solution (in triplicate) and prepared for aseptic technique. Atropine (about 0.02-0.07 mg/kg IM) was administered once before surgery to prevent bradycardia. Prophylactic antibiotics were given perioperatively at the time of surgery. For example, Excede® (ceftiofur crystalline free acid) at 2.5 mg/lb or other appropriate antibiotic treatment was administered to the donor.

Surgery. Aseptic surgical procedures were followed for all surgeries within this protocol.

### Procedure for Thymus Cells Collection

A sterile drape was placed over the donor (i.e., the piglet). With the piglet in dorsal recumbency, a longitudinal incision in the root of the neck (i.e., the suprasternal region) was made with a sterile blade. The tissue was retracted to observe the thymus and surrounding structures.

The piglet underwent a partial thymectomy using a combination of blunt and sharp dissection. Once the thymus was isolated, the vasculature was ligated, and the thymus was removed from the neck.

The incision was closed with appropriate suture material. The thymus was cut into small pieces of about 4 mm under aseptic conditions. A portion was retained for histological examination.

### Procedure for Placing Thymus Pieces in Kidney Capsule

In this example, the maceration step lasted for about 1 hour to ensure that the While still anesthetized, the piglet was placed in the lateral position. An about 5-8 cm long incision (horizontal or vertical at the surgeon's discretion) extending from the last rib into the paralumbar fossa and over the kidney was made. The caudal pole and lateral aspect of the kidney was mobilized and exposed within Gerota's fascia, taking care to protect surrounding structures.

A small incision was made in the kidney capsule, and a pocket was carefully made in a plane between the capsule and renal parenchyma. The previously prepared thymus pieces were injected under the capsule in an area approximately covering one surface of the caudal renal pole. The kidney capsule was closed with a Prolene 6-0 suture or edges of the kidney capsule incision was cauterized.

After ensuring hemostasis, the incision was closed with appropriate suture material. The procedure was repeated on the opposite kidney. The piglet was allowed to recover fully in surgical/recovery areas prior to returning to pens.

The thymus pieces were then allowed to mature in the donor for about 6-8 weeks before procurement for xenotransplantation.

### Procedure for Uthymokidney Procurement

The midline abdominal surgical site was prepared by removal of hair, followed by three alternating cycles of surgical scrub (chlorhexidine or betadine scrub) alternated with 70% alcohol or sterile saline. Using the scalpel and the electrocautery a midline incision was performed. The peritoneum was entered, and the contents were explored to rule out any abnormalities. Depending upon whether the right or left kidney was being removed, either the cecum and hepatic flexure or the descending colon/sigmoid and splenic flexure was taken down and cauterized reflecting the bowel medially.

The contents of the abdomen was packed, and a retractor placed to expose the retroperitoneum overlying the kidney. The renal pedicle was identified, and the renal artery and vein were dissected free from surrounding tissue. The vessels were isolated. The upper pole of the kidney was separated from the adrenal and surrounding tissue and the kidney was fully mobilized. The gonadal vein and ureter was identified and elevated, carrying a line of dissection caudad, medial to the ureteral/gonadal vascular bundle to the pelvic inlet where the ureter was transected, and the gonadal vein was tied and cut.

The pig was given 200-500 IU/kg of heparin IV and received 12.5 grams of mannitol, and 10 mg of Lasix. With the kidney completely mobilized, the pedicle was divided. The renal artery was divided first with a vascular stapler. Then the renal vein was divided in a similar manner.

The kidney was removed and placed in sterile ice and saline slush. The staple lines were excised, and the renal artery was flushed with 250-500 ml of University of Wisconsin (UW) solution. The kidney was prepped on the back table to prepare for implantation. The kidney was then placed in 200 ml of UW solution in a sterile bag and then wrapped inside two additional sterile bags and packed in ice for transport.

The pig was then euthanized immediately after the kidney was collected.

### Example 5: Thymolung Generation

This example demonstrates the feasibility of transplanting autologous thymus tissue into a lung parenchyma of the left superior lobe. Specifically, these experiments were performed: (1) to assess the feasibility of creating an intrapulmonary cavity that is large enough to house 3-5 g of thymus tissue; (2) to evaluate potential bleeding complications and cavity rupture risk; and (3) to assess the feasibility of an air-tight lung cavity closure after thymus transfer.

### Surgery and Animal Preparation

Two procedures (FIGS. 8A and 8B) were used to measure the oxygen uptake under varying blood flow conditions pre-terminally in animals (i.e., pigs). The animals (e.g., A661-9 (SKO, 35.1 kg) and A663-9 (SKO, 40.3 kg)) were used under an approved Institutional Animal Care and Use Committee (IACUC) protocol.

The animals (i.e., pigs) had been under deep general anesthesia for the initial experiment with routine mechanical ventilation settings, including a Positive end-expiratory pressure (PEEP) of 5 cmH2O. Both animals' chests were opened via medial sternotomy. The mediastinal thymic tissue was then dissected off the cranial pericardium and stored in sterile saline until use.

For the incision in the upper cranial left lung lobe, the PEEP was taken off (0 cmH2O). Carefully, the apical margin was incised with Metzenbaum scissors. While making sure to say within the core of the lobe tissue, a 2-inch deep cavity was created by spreading the branches of the scissors. A small peripheral air leak was observed in the second experiment. However, no relevant or significant bleeding was caused by the dissection.

The excised thymus tissue was cut into about one-inch (1″) long, and quarter-inch (¼″) diameter pieces. A scale was then used to weigh about 3.5 g of thymus tissue pieces which were introduced with forceps into the cavity. After all tissue pieces had been placed in the cavity, a TA™ auto stapler device was used on the cavity opening to close the incision.

FIG. 8A shows the transplantation of thymic tissue pieces into the left upper lobe of a lung parenchyma with the intra-parenchymal cavity containing thymus tissue and the airtight cavity stapler closure from the first experiment. FIG. 8B shows the transplantation of thymic tissue pieces into a lung pleura with the sub-pleural cavity containing thymus tissue and the airtight cavity stapler closure from the second experiment.

To test the airtightness of the stapler closure, a water test was performed. Specifically, a PEEP of 5 cmH2O was resumed and saline was drippled on the closure line. No air leak was observed in either animal. The experiment was terminated after the confirmation of the procedure feasibility.

After allowing 4-6 weeks of tissue engraftment, the thymolung will be explanted and used for histologic analyses. In addition, chest x-rays and/or CT scans can be performed to evaluate if the transplanted tissue can be imaged and distinguished from the surrounding pulmonary tissue to predict viability of the tissue.

This example shows that the transplantation of thymic tissue into the lung parenchyma of the left upper lobe was possible and feasible. During the two experiments, no complications, such as bleeding or air leak were observed. The example also demonstrates for the first time that the engraftment and function of the thymic tissue in the lung, a very delicate organ, is a viable and feasible surgical procedure. Thus, the results of this example established the engraftment and function of the thymic tissue in this anatomic location (i.e., thymolung) as an alternative to Thymo Kidney Transplantation (TKT).

### Example 6: Robotic Auto Thymo Kidney Transplantation

This example demonstrates the feasibility of a robotic auto ThymoKidney transplantation using a robot, such as da Vinci® Xi™ Surgical System or the da Vinci SP® Surgical System (Intuitive Surgical, Sunnyvale, CA, USA).

### Transplant Surgery

The da Vinci® Xi™ Surgical System. The da Vinci® Xi™ Surgical System was tested first. The da Vinci® Xi™ Surgical System is a multiport version of the robot that requires 4 separate surgical ports. A 50 kg anesthetized pig was used for the experiment. The pig was placed in supine recumbency, and the ports of the da Vinci® Xi™ Surgical System were inserted slightly para-midline right, to have a better and safer access/view on the right kidney. After abdominal CO₂ insufflation, the camera and the instruments were introduced into the abdomen.

The thymus was prepared. Specifically, a thymic lobe was excised from the pig's neck and cut into small pieces for the implant.

The robot was used to open the peritoneum and dissect the sub-peritoneal tissue to gain access to the kidney capsule. The kidney capsule was relatively easy to incise with the da Vinci® Xi™ Potts scissors. The capsule was elevated and separated from the underlying tissue using Maryland forceps.

Thymus tissue pieces were placed in a cut off glove fingertip and introduced into the abdomen through an instrument port. It was possible without any issue to transfer the thymus tissue pieces into the crated sup-capsular cavity. The capsule incision was then closed with a 6-0 multifilament suture.

Before closing the pre-renal peritoneum incision, the procedure was repeated twice more on other locations on the right kidney. FIG. 9 shows an image of a peritoneum incision following a Robotic auto ThymoKidney Transplantation (TKT) using the da Vinci® Xi™ Surgical System. FIG. 9 further illustrates the thymus under kidney capsule and the closed kidney capsule incision post TKT.

This example demonstrates for the first time that Thymokidney can be generated at more than one location using a single incision. For example, the Thymokidney can be generated at one location, 2 locations, 3 locations, 4 locations, 5 locations, 6 locations, or more locations using a single surgical incision.

The da Vinci SP® Surgical System. The surgical procedure was repeated using the DaVinci SP® Surgical System. The DaVinci SP® Surgical System only requires a single surgical access for the camera and instruments. The incision was made in the umbilicus. Moreover, the incision was about three times larger in diameter than each of the required da Vinci® Xi™ incisions. In addition, a separate access port was installed to pass sutures, tissue, etc. into the abdomen. However, both kidneys were readily accessible from this midline incision.

Opening the kidney capsule was also not possible with the da Vinci SP® scissors as those were not as pointy and sharp on the tip as the da Vinci® Xi™ Potts scissors. Two alternative methods for the incision and both tested methods worked successfully. First, a suture needle was used to slightly elevate the capsule and then the capsule was incised in the elevated area with scissors. The tissue insertion generated using this method was identical to the he da Vinci® Xi™ technique. In the second approach, a hollow syringe needle was carefully inserted under the capsule. The needle was connected to a tubing to which a syringe was attached. 3 ml of air was then inflated under the capsule. This second technique separated the capsule in a large area from the underlying kidney tissue. After the cavity was created, the capsule was incised starting at the needle hole. Thymus tissue pieces were placed in a cut off glove fingertip and introduced into the abdomen through an instrument port. The closure of the incision and peritoneum was possible without problems.

### Conclusion

This example demonstrates that robotic auto ThymoKidney Transplantation (TKT) was possible with either the da Vinci® Xi™ Surgical System or the da Vinci SP® Surgical System. Access to either kidneys from the abdominal midline was possible as shown with the da Vinci SP® Surgical System and the SP access port. Furthermore, different techniques can be applied to incise the kidney capsule and elevate the capsule to create the cavity. For example, scissors, a suture needle, or a syringe needle can be used. This example also shows for the first time that air insufflation helped to create a large sub-capsular cavity for transplanting thymus tissue pieces. Long forceps-style instruments were also functionally able to insert the thymus tissue into the cavity. Lastly, closure of the capsule and peritoneum was possible with sutures.

In summary, Thymokidneys can be created using the Intuitive DaVinci robots. It seems reasonable that this new robotic surgery can be established as a routine procedure over time. The da Vinci® Xi™ Surgical System is a particularly suitable robot for TKT surgeries due to its size (e.g., height) and the wider range of available instruments.

### Example 7: a Thymokidney Graft Supported Human Thymopoiesis

Xenotransplantation could overcome the inadequate supply of human organs. Building on preclinical studies, an alpha-Gal knock out porcine composite thymus-kidney (thymokidney) transplant was generated, for the possibility of inducing tolerance in a 54-year-old woman with diabetes-associated renal failure and advanced heart failure from ischemic cardiomyopathy. She was not a candidate for a heart or kidney allotransplant due to exclusionary co-morbidity. She received a left ventricular assist device 8 days prior to the xenotransplant. Immunosuppression included T cell, B cell and immunoglobulin depletion and ongoing tacrolimus, mycophenolate mofetil, belatacept, corticosteroids and complement inhibition. After 1 month of normal renal function, irreversible ischemic injury occurred due to inadequate renal perfusion in the setting a septic episode and the xenograft was explanted on post-operative day (POD) 47. She was later transferred to hospice and expired on POD86.

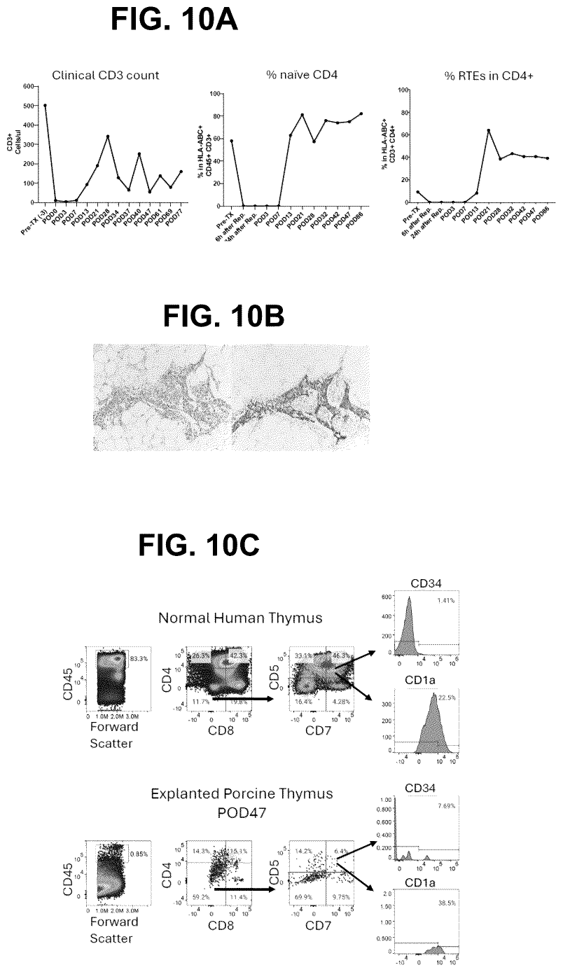

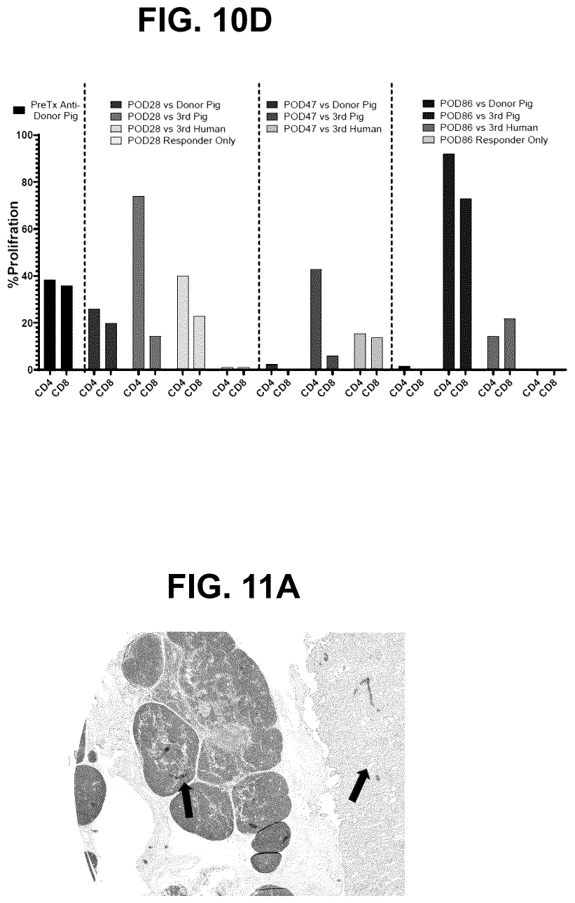

Peripheral blood T cell concentrations declined to a nadir of 10/ul following induction therapy and began to recover on POD13 toward a peak of 342/μl by POD28. Sixty-four percent (64%) of CD4 T cells recovering by POD21 had the “naïve” phenotype (CD45RA⁺CCR7⁺) and expressed CD31, suggesting that they were recent thymic emigrants (RTEs). In contrast, pre-transplant CD4 T cells included <10% RTEs (FIG. 10A).

The patient's age, prolonged illness and two sternotomies made the recovery of RTEs unexpected. Histology from the porcine thymokidney explant revealed thymic cytokeratin-positive epithelial cells forming a network closely associated with lymphocytes (FIG. 10B). Flow cytometric analysis on isolated thymic single cells revealed human CD45⁺ leukocytes that included CD4⁺CD8⁺ (double positive), single positive, and double negative thymocytes. The double negative population in the porcine thymus included subsets expressing CD5 and CD7, as well as CD1a and CD34, consistent with T cell progenitor populations found in a normal human thymus (FIG. 10C).

A mixed lymphocyte reactions was established by co-culturing fluorescent cytoplasmic dye-labeled T cells from patient blood with irradiated donor pig dendritic cells. Specific proliferative hyporesponsiveness toward the donor pig with preserved responses to third party pig and allogeneic human evolved over time, with complete donor-specific unresponsiveness by POD47 and POD86 more than a month after stopping all immunosuppression (FIG. 10D).

Collectively, these results show for the first time that the porcine thymic component of the xenotransplant supported human thymopoiesis and that these T cells, which were specifically tolerant toward the donor pig, entered the circulation. These results established that the thymokidney described herein can be used for human T cell tolerance induction to porcine xenografts. Moreover, the thymokidney could replace high levels of chronic immunosuppression.

### Example 8: a Composite Thymokidney Transplant Tolerized Xenogeneic Recipients to the Porcine Source Animal

Introduction. Porcine thymic transplantation tolerized xenogeneic recipients to the porcine source animal in animal models. A 54-year-old woman with complex comorbidities received a composite thymokidney transplant from an α-GalT knockout pig, achieving initial renal function before graft failure for non-immunological reasons on postoperative day (POD) 47.

Methods. Serial peripheral blood mononuclear cell (PBMC) samples were analyzed by spectral flow cytometric (FCM), and mixed lymphocyte reactions (MLRs) were performed on purified T cells to assess tolerance. Thymocytes were isolated from the thymic portion of the graft explant and analyzed using spectral FCM.

Results. T cell levels in peripheral blood declined to 10/μl post-induction therapy and recovered to a peak of 342/μl by POD28. By POD21, 64% of the recovering CD4 T cells displayed a CD45RA⁺CCR7⁺ “naïve” phenotype and expressed CD31, consistent with recent thymic emigrants (RTEs). Pre-transplant CD4 T cells included <10% RTEs. FCM analysis on cells from the POD47 thymic graft revealed human CD45⁺ leukocytes (0.85%), including double-positive (CD4⁺CD8⁺), single-positive, and double-negative thymocytes. The double-negative cells expressed markers, such as CD5, CD7, CD1a, and CD34, consistent with human T cell progenitors in the porcine thymus.

MLR results demonstrated progressive hyporesponsiveness toward the source pig with preserved responses to third-party pig and allogeneic human cells, achieving full donor-specific unresponsiveness by POD47 and POD86.

Conclusion. These data show that a porcine thymus in a thymokidney graft supported human thymopoiesis and the development of T cells tolerant to the pig. Furthermore, these data show for the first time that thymic transplantation is a promising approach to achieving T cell tolerance in xenotransplant recipients.

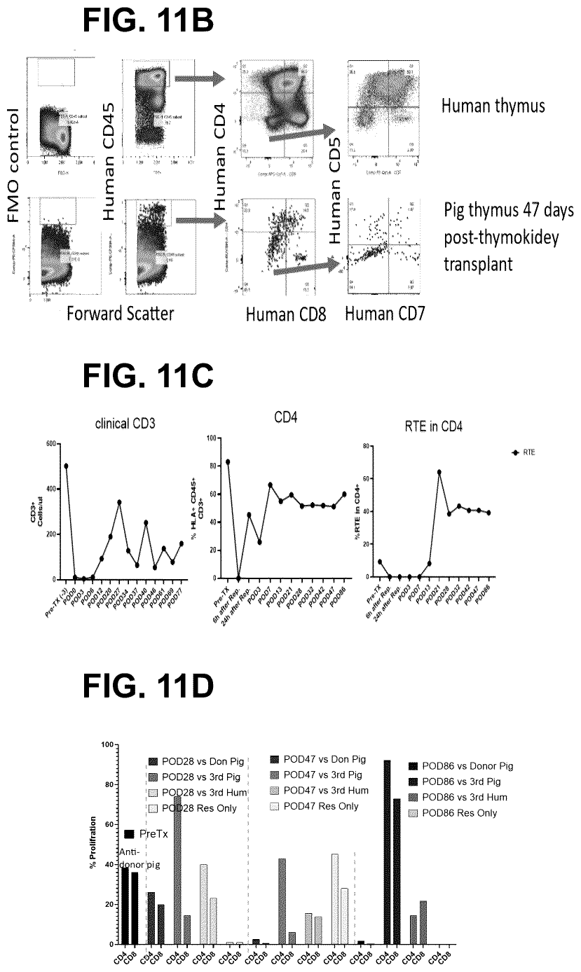

Summary. The UThymo™ kidney (i.e., thymokidney) contains subcapsular porcine thymus. Histologic evidence demonstrates reconstitution of the thymic tissue under a kidney capsule (FIG. 11A). Importantly, the UThymo™ kidney living recipient whose kidney xenograft lost function due to hypoperfusion in the setting of a left ventricular assist device afforded the opportunity to evaluate the thymus of the UThymo™ kidney explanted xenograft. Evidence of recipient thymopoeisis in the porcine thymus was found (FIG. 11B). Moreover, peripheral blood T-cells showed marked enrichment for recent thymic emigrants (RTEs) following xenotransplant compared to pre-transplant (FIG. 11C) and mixed lymphocyte reaction (MLR) assays demonstrated donor-specific unresponsiveness with robust third party responses (FIG. 11D), suggesting that the recovering RTEs developed in the porcine thymus xenograft and achieved tolerance (as described in Examples 7 and 8). These data demonstrate that the UThymo™ kidney achieved immune tolerance.

### Example 9: Donor-Reactive T Cells and Innate Immune Cells Promote Pig-to-Human Xenograft Rejection

Kidney transplantation is the only definitive treatment for end stage renal disease (ESRD), but the number of available organs falls far short of demand. Xenotransplantation, the transplantation of organs across species, offers a potential solution to this shortage. Pigs are a particularly attractive source animal candidate, given their suitable organ size, physiology, and modifiable genetics.

Both adaptive and innate immune responses pose barriers to xenotransplantation. In adaptive responses, T cells recognize xenoantigens both through the direct pathway, in which recipient T cells recognize donor antigens on donor antigen-presenting cells (APCs), and through the indirect response, in which xenoantigens are processed and presented by recipient APCs. The indirect pathway has been reported to be more potent for xenoresponses than alloresponses. The innate xenoresponse includes natural antibodies that can cause hyperacute or delayed antibody-mediated rejection when directed against pig carbohydrate antigens. In addition, natural killer (NK) cells are prominent components of the cellular infiltrates in xenografts, potentially because pigs do not express inhibitory ligands that prevent human NK cell activation.

Preclinical xenotransplantation studies have largely relied on non-human primates (NHPs). Although quite valuable, studies in these animals may not fully replicate the human xenoresponse. The pig-to-decedent human model has allowed investigation of these responses, primarily in short-term (up to 7 days) studies. Recently, a longer-term study was carried out in the pig-to-decedent model that allowed unprecedented investigation of the physiological and immunological outcomes of pig kidney transplantation for 61 days in a human body.

The data shown in this example showed that in the xenotransplantation setting, pig thymus induced central tolerance to the porcine donor of recipient αβ T cells, resulting in xenograft tolerance. Moreover, the immune response in the above brain-dead human recipient transplanted with an alpha-galactosyltransferase (α-Gal) knockout (KO) pig composite thymokidney graft and followed for 61 days. A method was established to first identify human anti-pig xenoreactive T cell clones. Then T cell reactive clones (XDRTCCs) were tracked in the decedent's peripheral blood and kidney biopsies. The data show that pig-reactive T cell clones (XDRTCCs) selectively expanded in the periphery following the transplant and were detected in a perinephric lymphocele as early as post-operative day (POD) 14, where they demonstrated an early activated transcriptional profile. XDRTCCs detected later in the kidney graft during an episode of acute antibody-mediated rejection (AMR) demonstrated mixed effector and exhausted transcriptional profiles. Infiltrates of effector γδ T cells and NK cells with marked effector function were also detected in the kidney biopsy. This is the first comprehensive analysis of the innate and adaptive cell-mediated rejection response in pig to human xenotransplantation. The results described herein have important implications for immunosuppression design and highlight the need to better suppress T cell responses.

### Results

a. Establishing direct and indirect Mixed Lymphocyte Reactions (MLR) to identify xenoreactive T cell clones

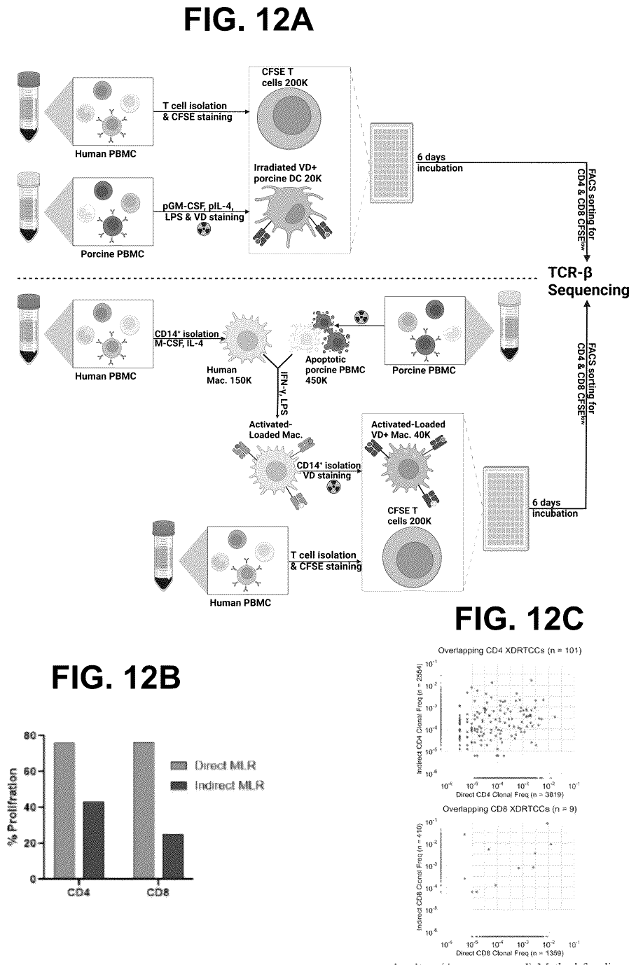

To study the human anti-pig indirect and direct T cell response, in vitro assays were first established. For the direct response, peripheral blood mononuclear cells (PBMCs) of an α-GAL KO SLA-HH pig were plated and the adherent cells were used for dendritic cell (DCs) differentiation. These porcine DCs were co-cultured for 6 days with CFSE-labelled sorted CD3+ human T cells purified (>90% purity) from a healthy donor (FIG. 12A, upper panel). As shown in FIGS. 12B, 75.9% and 76.4% proliferation of CD4 and CD8 T cells, respectively, at the end of the coculture period.

To set up the indirect response (FIG. 12A, lower panel), human T cells were purified (>90% purity) and human macrophages were differentiated from the same healthy volunteer. Macrophages were activated and loaded with pig antigens. Activated and loaded macrophages were co-cultured with CFSE-labelled T cells and induced 43.2% and 25.1% proliferation of CD4 and CD8 T cells, respectively (FIG. 12B).

Semi-direct presentation of intact donor MHC-peptide complexes on recipient APCs is an additional pathway that has been described for alloresponses. If such complexes were picked up and presented intact by recipient macrophages in the indirect MLR described above, they might be expected to expand the same T cell clones as those activated by direct presentation on porcine donor APCs. Therefore, bulk TCRB CDR3 sequencing was performed on the T cell response from each pathway to determine the extent of clonal overlap. As shown in FIG. 12C, most clones appeared along the y (indirect) or x (direct) axis in the frequency correlation plots, indicating that these clones were unique to one or the other response. In the CD4 response, only 101 overlapping clones were detected between 3,819 direct and 2,554 indirect clones (FIG. 12C, upper panel). In the CD8 response, only 9 overlapping clones were detected between 1,359 direct and 410 indirect clones (FIG. 12C, lower panel). A similar minimal level of overlap between directly and indirectly xenoreactive clones was observed in a repeat experiment. Taken together, these data demonstrate that these indirect and direct assays identify largely distinct, non-overlapping xenoreactive T cell repertoires.
b. TCRB CDR3 sequencing demonstrates expansion of T cell clones in circulation of human decedent recipient of porcine thymokidney transplant

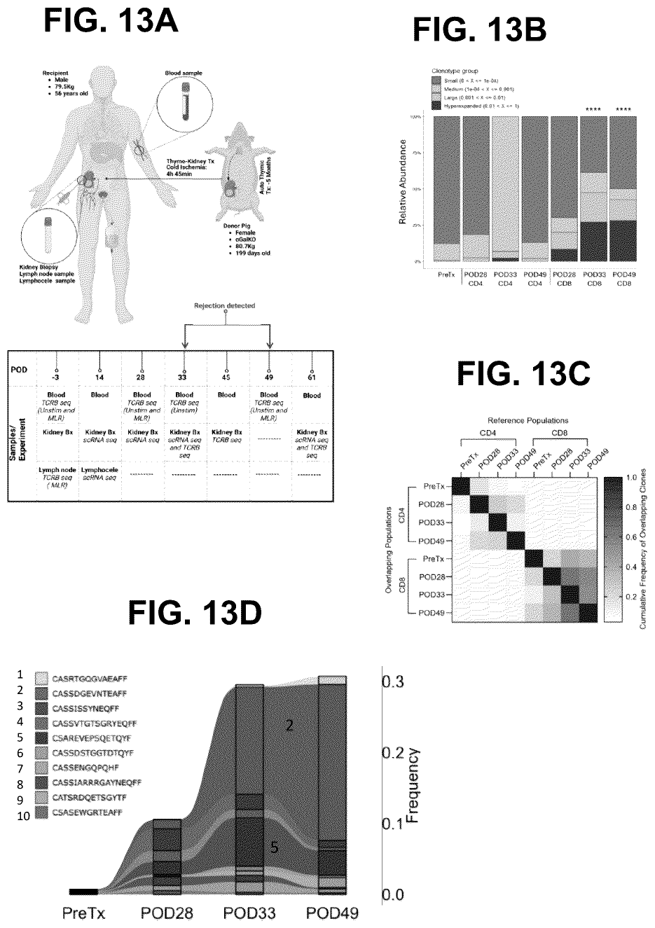

A porcine thymokidney graft was constructed by autologous thymus transplantation under the kidney capsule of an α-GAL KO source pig (FIG. 13A). As described herein, in the xenotransplantation setting, pig thymus induced central tolerance to the porcine donor of recipient αβ T cells, resulting in xenograft tolerance. After 5 months, the thymokidney was transplanted into a brain-dead, decedent recipient who had undergone bilateral native nephrectomy. After successful thymokidney transplantation, the decedent recipient underwent frequent peripheral blood and kidney biopsy sampling at various time points (FIG. 13A). Throughout the study, the thymokidney demonstrated life-sustaining physiological function, supporting normal hemodynamics and electrolyte balance. Induction and maintenance immunosuppression are summarized in Table 1. Complement deposition gradually increased in the tissue, culminating in an episode of biopsy-proven AMR on POD33. This episode was treated with 5 alternate-day plasmaphereses along with the C3 inhibitor pegcetacoplan (Table 1). Serum creatinine rose again at POD49 after concluding these treatments, and repeat biopsy showed ongoing AMR. Another cycle of 5 plamapheresis treatments and rATG was shown to have completely reversed the AMR on a repeat biopsy on POD56. The creatinine returned to baseline and the xenograft remained free of rejection until termination of the study on POD61.

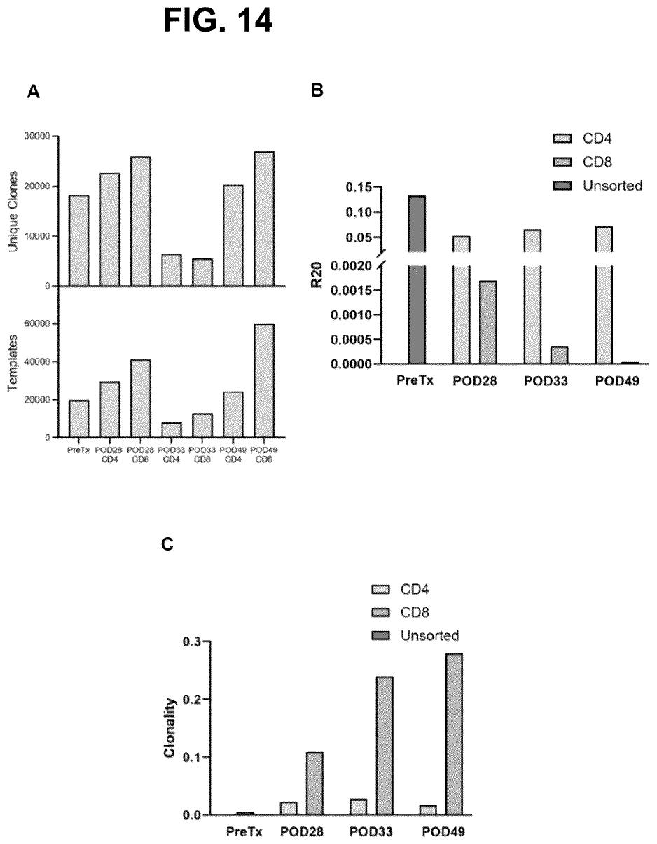

Circulating T cell clones were monitored in the decedent recipient by performing high-throughput TCRB CDR3 sequencing on peripheral blood T cell DNA extracted pre-transplant and at several post-transplant timepoints. The number of templates (i.e., total TCRB CDR3 sequences obtained) and unique clones identified in each sample are summarized in FIG. 14A. Over time, there was an increase in the abundance of hyperexpanded clonotypes, defined as clones with greater than 1% frequency. At POD33 and POD49, CD8 hyperexpanded clonotypes accounted for more than 25% of the CD8 repertoire, a statistically significant increase from the pre-transplant baseline. (FIG. 13B). Among CD8 T cells, the R20, the proportion of clonotypes representing 20% of templates, decreased (FIG. 14B), while clonality, whereby higher scores indicate less clonal diversity, increased (FIG. 14C). The cumulative frequency of overlapping clones confirmed that the same immunodominant CD8 clones were detected at each post-transplant timepoint (FIG. 13C). For instance, the same clones that made up 63% of the POD33 CD8 repertoire made up 52% at POD49. The top 5 expanded clones made up only 1% of the pre-transplant CD8 repertoire compared to more than 30% by POD49 (FIG. 13D). One clone in particular, shown as #2, comprised 0.1% of the pre-transplant repertoire and expanded to over 20% of the repertoire by POD49. Thus, marked expansion of a few immunodominant CD8 clones occurred over time post-transplant. FIG. 14B and FIG. 14C illustrate a more modest and non-progressive loss of diversity among post-transplant CD4 clones compared to pre-transplant T cells.
c. Selective post-transplant expansion of XDRTCCs

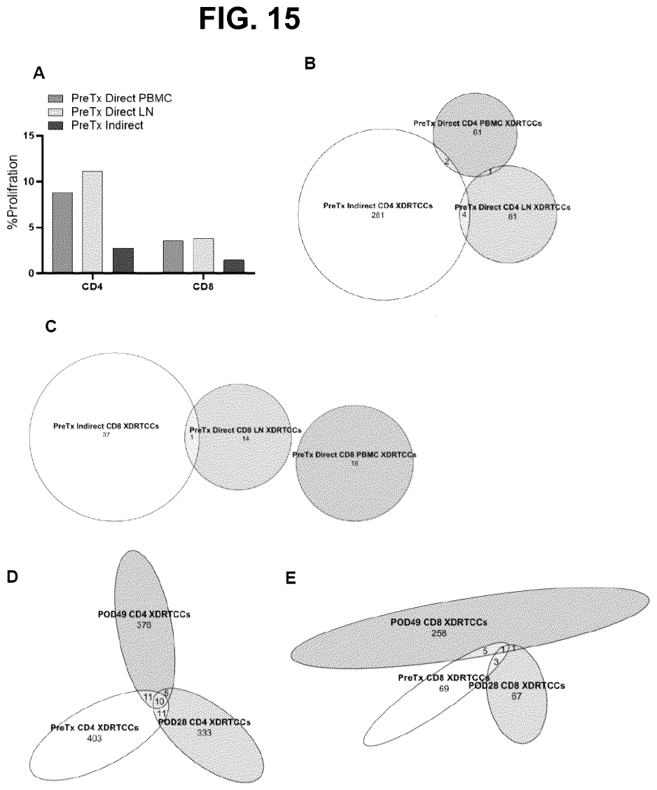

The method for identifying directly and indirectly xenoreactive T cell clones in vitro described herein was used, and then XDRTCCs were tracked in the long-term human decedent recipient of a porcine thymokidney graft. Using pre-transplant PBMCs from the decedent, direct and indirect MLRs against the source pig was set up. Unfortunately, cell clumping of the decedent PBMCs precluded the identification of large numbers of clones in stimulated and unstimulated cell populations. In order to identify additional XDRTCCs, therefore, similar studies were performed using a lymph node obtained pre-transplant and using post-operative PBMCs as responders. The latter included a direct MLR using sorted CD3+ cells from POD28 recipient PBMCs and another with POD49 recipient PBMCs (FIG. 15A). CFSElow CD4+ and CD8+ T cells from each MLR were sorted for TCRB CDR3 sequencing. Because XDRTCCs identified post-transplant and not pre-transplant are likely to have already expanded in vivo in response to the xenograft, the criteria for their identification included a comparison to their frequency in pre-transplant rather than simultaneous post-transplant unstimulated populations. To exclude bystander proliferation effects and sorting errors, only clones with a frequency in the divided population at least twice that of their pre-transplant unstimulated frequency were considered XDRTCCs (Table 2). The indirect and direct pathways of xenostimulation expanded largely non-overlapping XDRTCCs (FIGS. 15B-E). In total, between the pre-and post-transplant MLRs, 1,058 CD4+ and 386 CD8+ XDRTCCs (Table 2) were identified.

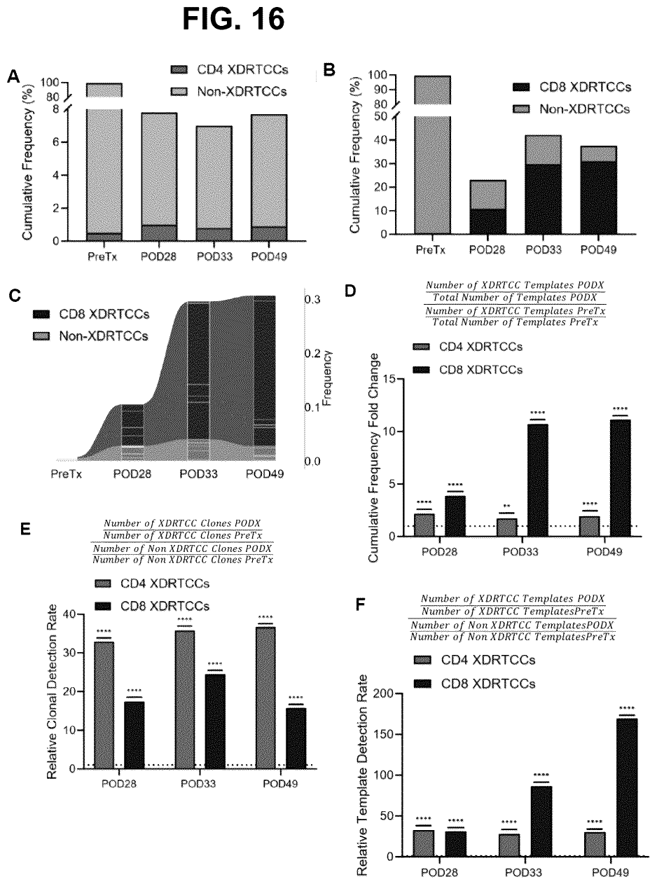

The longitudinal post-transplant PBMC samples were compared for the frequency of XDRTCCs. CD4 XDRTCCs comprised less than 2% of the total templates in the post-transplant peripheral blood populations but still demonstrated an increase above the pre-transplant baseline (FIG. 16A). CD8 XDRTCCs demonstrated more striking expansions, constituting 10% of the total CD8 repertoire at POD28 and around 30% at POD33 and POD49, both of which were established rejection timepoints (FIG. 16B). Individual expanded XDRTCCs could be traced across multiple time points, with one single clone comprising over 20% of the repertoire at POD49 (FIG. 16C). Indeed, this highly expanded XDRTCC proved to be the same clone as the most expanded clone (shown as #2) in FIG. 13D and was initially identified in the POD28 direct MLR as well as in the PBMC versus PBMC POD49 MLR. All 5 top clones in FIG. 13D were, in fact, XDRTCCs. Normalizing the total quantity of XDRTCCs in the post-transplant periphery to their pre-transplant baseline revealed clear XDRTCC enrichment, with increases reaching up to 12-fold at POD49 for CD8 cells (FIG. 16D).

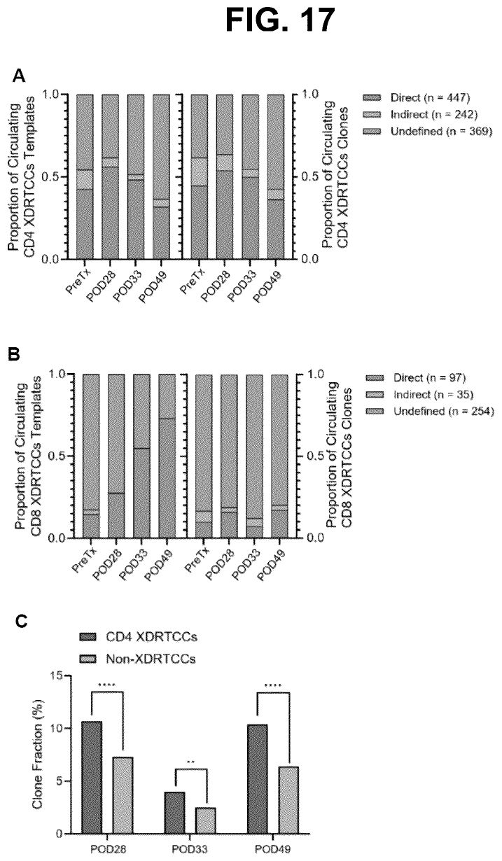

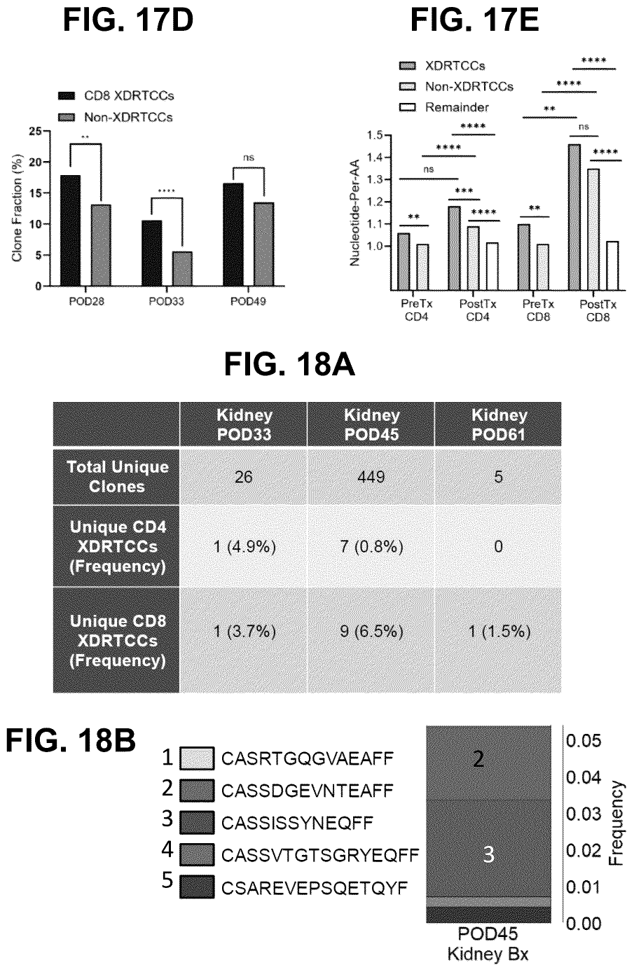

Whether the circulating XDRTCCs detected at each timepoint were directly or indirectly xenoreactive or were undefined was also assessed. The analysis was performed because the circulating XDRTCCs were either expanded in both responses or only detected in the POD49 PBMC versus PBMC MLR. At all timepoints, a substantial proportion of detected CD4 XDRTCC clones were directly xenoreactive and a smaller proportion was indirectly reactive. Additionally, significant numbers of undefined templates and clones were also detected (FIG. 17A). The proportion of CD8 XDRTCC templates from the direct response increased from 15% pre-transplant to 73% by POD49. This increase reflected XDRTCC clonal expansions, as the proportion of donor-reactive unique clones that were directly reactive did not increase (FIG. 17B). Only a minor portion of XDRTCC CD8 unique clones and templates was indirectly reactive and a considerable portion of each was undefined (FIG. 17B). The increasingly dominant directly reactive XDRTCC CD8 expansion was mainly driven by massive expansion of the #2 clone in FIG. 13D.

Since clonal expansions may have been induced in part by the lymphopenia produced by induction treatment with rATG prior to transplant, the expansion of XDRTCCs was compared to that of non-XDRTCCs, which were defined as clones present in the pre-transplant blood but not in any proliferated MLR population. Comparing the rates of post-transplant versus pre-transplant XDRTCCs and non-XDRTCC detection, XDRTCC-specific expansion was readily apparent at both the clonal (FIG. 16E) and template levels (FIG. 16F). These relative expansions (up to ~175-fold for CD8 XDRTCCs at POD 49) were most striking when calculated at the template level, in which each copy of an individual clone is given equal weight. The actual proportion of XDRTCC clones detected post-transplant was statistically significantly greater than that of non-XDRTCCs for both CD4s and CD8s, substantiating the XDRTCC-specific expansion (FIG. 17C-D). Moreover, amongst TCRs in the post-transplant peripheral blood, a greater average number of CD8 XDRTCC unique nucleotide sequences converged to the same amino acid sequence than was observed pre-transplant, suggesting that specific antigens were driving expansion of donor-reactive clonotypes following the transplant (FIG. 17E). While the same trend applied to CD4 XDRTCCs compared to non-XDRTCCs, it did not reach significance. Following the transplant, the average nucleotide to amino acid sequence ratio also increased significantly for non-XDRTCCs among CD4 and CD8 subsets, consistent with antigen-driven expansion of both sets of clones during post-transplant lymphopenia.

In contrast, clones that were identifiable as neither XDRTCCs nor non-XDRTCCs post-transplant maintained a nucleotide to amino acid sequence ratio close to 1 (shown as “remainder” in FIG. 17E). Moreover, analysis with GLIPH2 revealed the presence of clones sharing similar CDR3Bs with XDRTCCs, indicating a higher likelihood of recognizing the same antigen. CD4 XDRTCC-like clones accounted for 79% as many templates as CD4 XDRTCCs in the post-transplant circulation, while CD8 XDRTCC-like clones accounted for 3% as many templates as CD8 XDRTCCs. The immunodominance of the CD8 XDRTCC population suggests that the MLRs successfully identified the most xenoreactive clones.

Conversely, non-XDRTCC-like clones accounted for 73% and 50% as many templates as non-XDRTCCs in the CD4 and CD8 post-transplant populations, respectively. This result aligned with expectations of antigen-driven proliferation. In sum, the studies demonstrated marked and antigen-driven expansion of XDRTCCs and, to a lesser extent, of non-XDRTCCs in the circulation following pig-to-decedent human xenotransplantation.

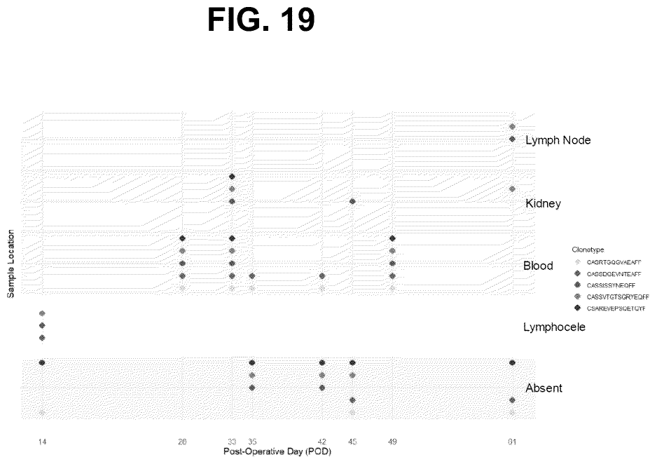

For each of POD 33, 45, and 61, two 10 um-thick sections of the formalin-fixed, paraffin-embedded (FFPE) xenograft biopsy specimens were cut and submitted for mRNA-based TCRβ recovery (RepSeq+ assay, iRepertoire, Inc). The number of unique TCR CDR3B RNA transcripts retrieved is summarized in FIG. 18A and XDRTCCs were detectable in all biopsies, including two on POD33, sixteen on POD45 and one on POD61. Among the nine unique CD8 XDRTCCs in the POD45 biopsy, five clones matched the immunodominant circulating CD8 XDRTCCs identified in FIG. 13D and FIG. 16C. Their respective RNA frequencies in the biopsy are shown in FIG. 18B. Integration of all TCR data in our study across all samples and time points identified five clones that could be traced from the POD14 lymphocele, then blood, then kidney biopsy during the AMR episode, and then finally the study termination at POD61 (FIG. 19). These five XDRTCCs clones were immunodominant circulating CD8 XDRTCCs as shown in FIG. 13D and FIG. 16C.
d. Detection of XDRTCCs with effector function in the kidney biopsy during an antibody-mediated rejection episode

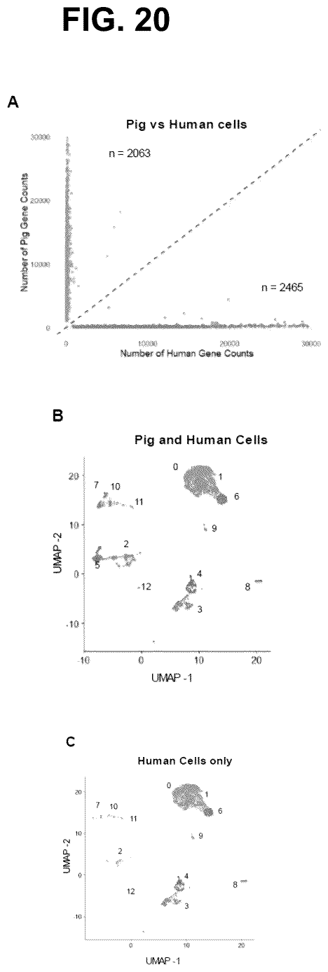

To further characterize T cells in the xenograft itself, leukocytes from kidney biopsies obtained at various time points (FIG. 13A) were isolated, MACS sorted them for human CD45⁺ cells and carried out single-cell RNA and TCR sequencing. Additionally, cells were collected from a lymphocele (POD 14L) that surrounded the transplanted pig kidney at POD14. The numbers of leukocytes submitted for single-cell RNA sequencing are summarized in Table 3. The number of pig and human transcripts in each cell were quantified. Most cells expressed almost entirely human or pig transcripts, making the species of origin for each cell easily distinguishable (FIG. 20A). All pig cells were removed from downstream analysis (FIGS. 20B-C).

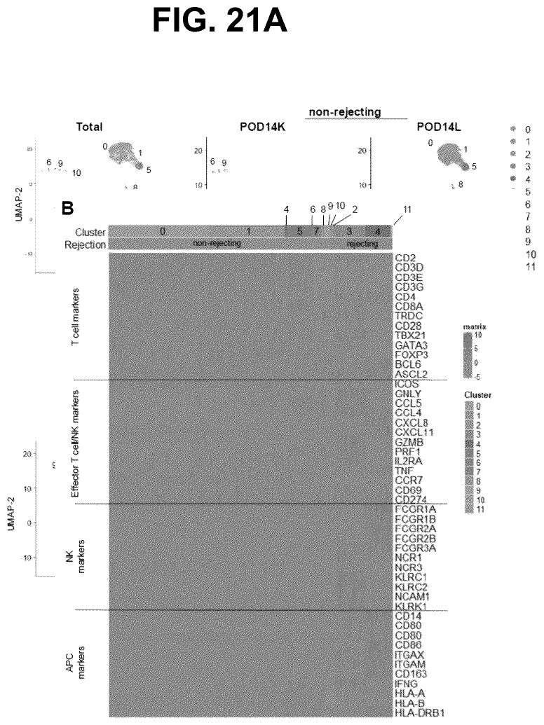

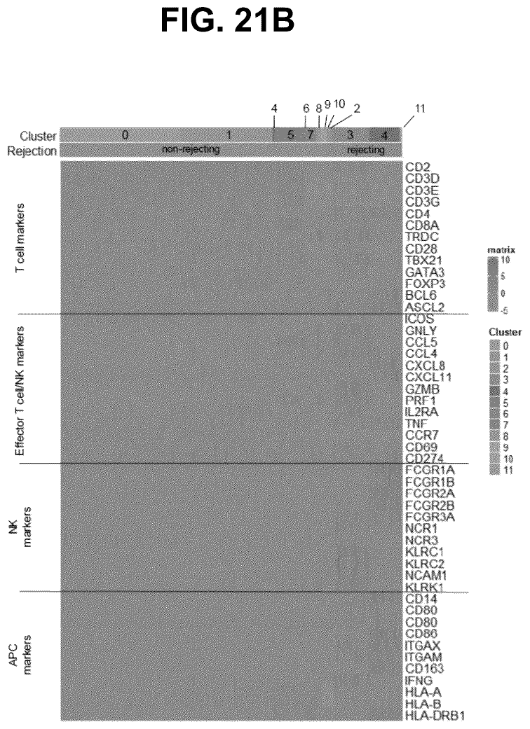

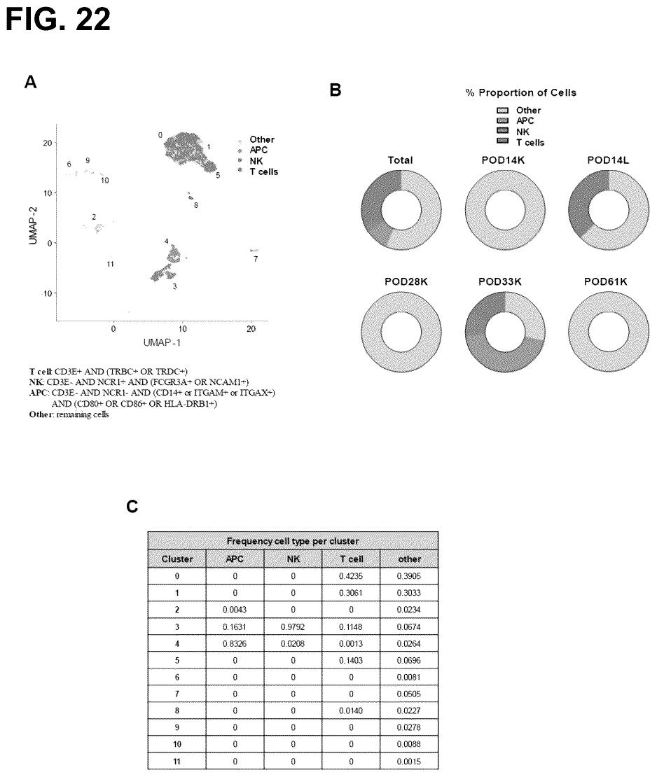

Single-cell RNA sequencing data from all samples were processed and integrated by the standard Seurat pipeline and projected on a single Uniform Manifold Approximation and Projection plot for visualization in two dimensions (FIG. 21A). To identify immune cells, the data were interrogated for immune cell markers (FIG. 21A, FIG. 21B, FIG. 22A, and FIG. 22B). FIGS. 22A-C show the cluster distribution of each cell type in the single-cell RNA sequencing analyses. As shown in the pie charts in FIG. 22B, the non-rejecting kidney biopsy specimens (POD14K, 28K, 61K) did not reveal gene expression patterns consistent with human T cells, NK cells, or APCs. In contrast, the POD14L and the POD33 rejecting kidney specimen both contained T cells. The rejecting kidney specimen included NK cells and activated APCs (FIGS. 21A-B and FIGS. 22A-B), RNA species characteristic of T cells (e.g., CD3E, CD4, CD8A, CD28), NK cells (e.g., FCGR3A, NCR1, NCAM1), effector T cells and effector NK cells (e.g., GZMB, PRF1, TNF, GNLY) and APCs (e.g., CD80, CD86, HLA-DRB1).

Specific clusters identified on the UMAP segregated with specific specimens. For example, Clusters 0, 1, 5, and 8, containing mainly T cells, were predominantly detected in POD14L, whereas Clusters 2, 3, 4, and 11, containing T cells, NK cells and APCs, were predominantly detected in the POD33 kidney biopsy, during the episode of acute humoral rejection (FIGS. 21A-B). Cluster 3, which was uniquely present in the rejecting sample (FIGS. 21A-B) and included both T cells and NK cells (FIG. 21B and FIG. 22A), demonstrated high expression of cytotoxicity genes, including GZMB, PRF1, TNF, and GNLY (FIG. 21B). Activated APC populations with upregulated costimulatory ligands, Fc receptors, chemokine receptors, HLA-DR, TNF and integrins were predominantly detected in Cluster 4 (FIG. 21B and FIG. 22A).

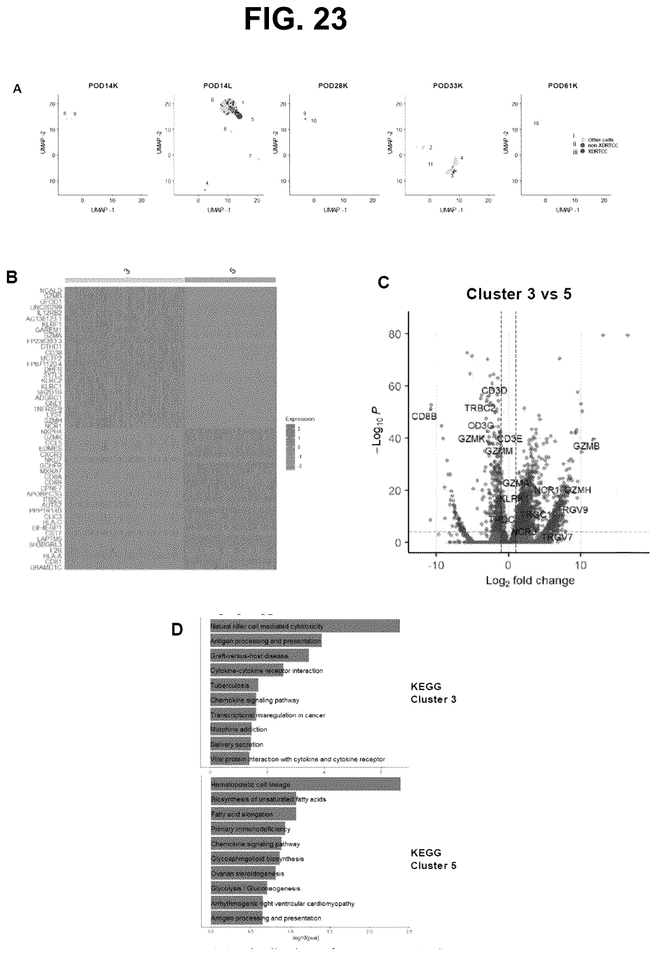

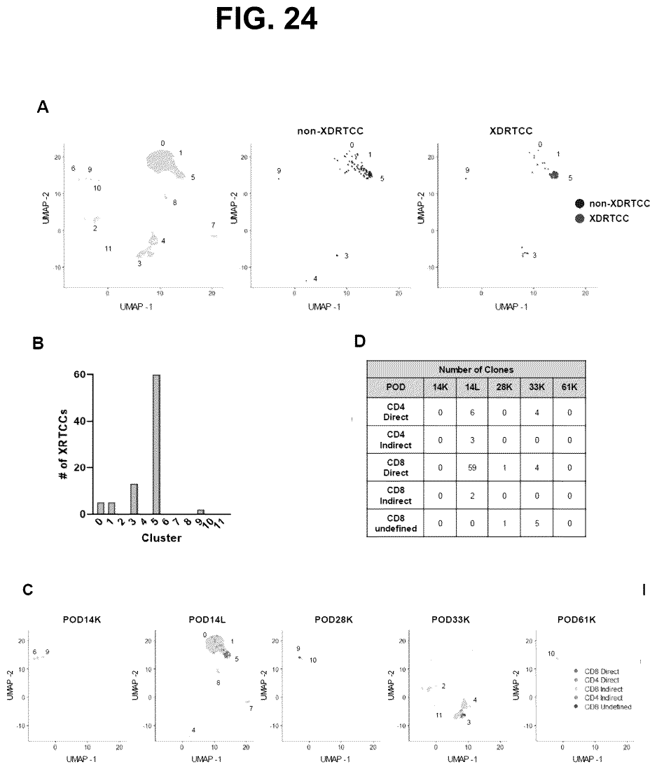

The single-cell TCR sequencing data for XDRTCCs were further analyzed. XDRTCCs were detected in the POD14L sample and kidney biopsy specimens from POD28 and POD33 only. The largest number of XDRTCCs was detected prior to rejection in POD14L in Cluster 5 (FIG. 23A and FIGS. 24A-B). Importantly, XDRTCCs in the rejecting specimen on POD33 were detected exclusively in Cluster 3 (FIG. 23A and FIGS. 24A-B), the major cluster containing cytotoxic T cells. The majority of XDRTCCs found in the lymphocele and kidney biopsy were directly xenoreactive CD8 T cells, although the lymphocele also included directly and indirectly xenoreactive CD4 cells and POD33K included directly donor-reactive CD4 clones and undefined CD8 clones (FIGS. 24C-D). Several clones within the POD33 kidney biopsy belonged to the most expanded clonotypes detected in the blood (FIG. 13D).

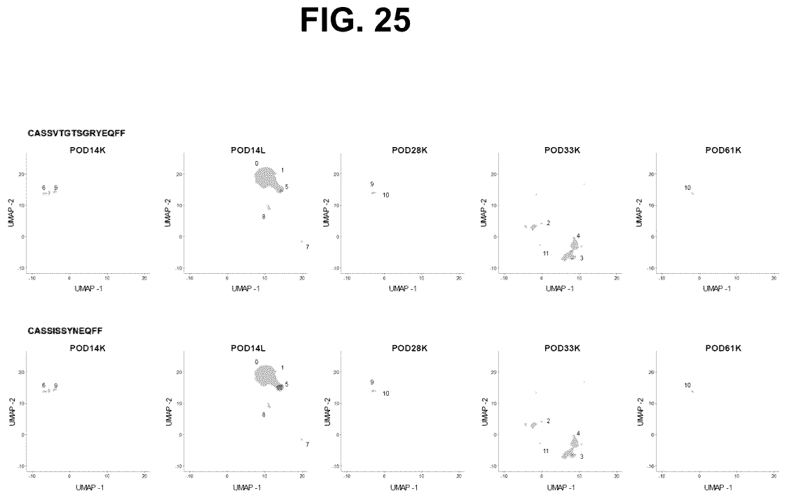

XDRTCCs in cluster 5 (present in POD14L sample) differentially expressed several markers of activation and effector differentiation, including GZMK, EOMES, and CCL5 (FIGS. 23B and 23C). Some of these XDRTCCs were later detected in cluster 3 in the biopsy at POD33, during the episode of acute rejection (FIG. 25), suggesting that initial T cell activation in lymphatic tissues preceded migration into the xenograft and rejection. Cluster 3, containing the thirteen XDRTCCs upregulated in the biopsy specimen during rejection at POD33 was compared to the main T cell-containing cluster, namely Cluster 5 in POD14L (FIG. 23A). Cluster 3 differentially expressed cytotoxic genes GZMA, GZMB, GNLY, PRF1 and GZMH as well as IFNG, while cluster 5 expressed GZMK (FIGS. 23B-C and FIG. 26). CD8B and TRBC2 were prominent in cluster 5, consistent with its αβ T cell content (FIG. 23C). In contrast, Cluster 3 also contained TRGC1, TRDC, and NCR1 (FIGS. 23B-C) in addition to the XDRTCC αβ TCRs mentioned above (FIG. 23A), suggesting that Cluster 3 included γδ T cells and NK cells as well. Indeed, using pathway analysis, the top upregulated pathway in Cluster 3 corresponded to NK cell-mediated cytotoxicity, suggesting a contribution of innate immune cells to cytotoxicity (FIG. 23D).

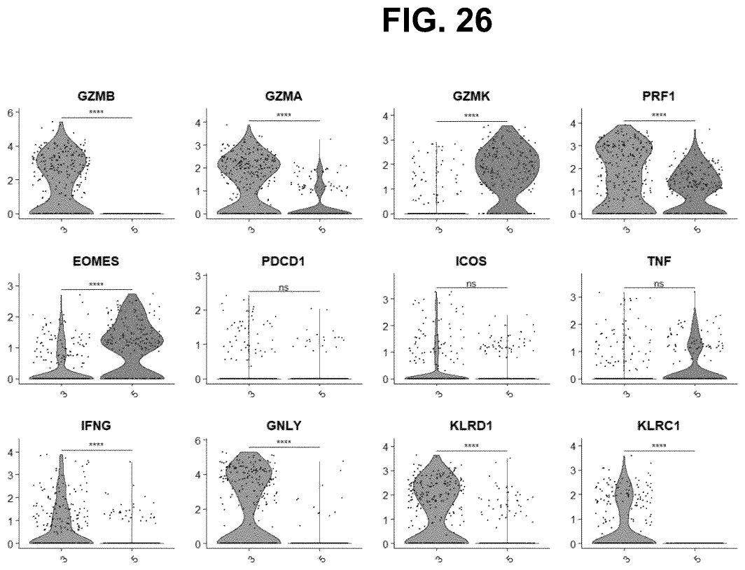

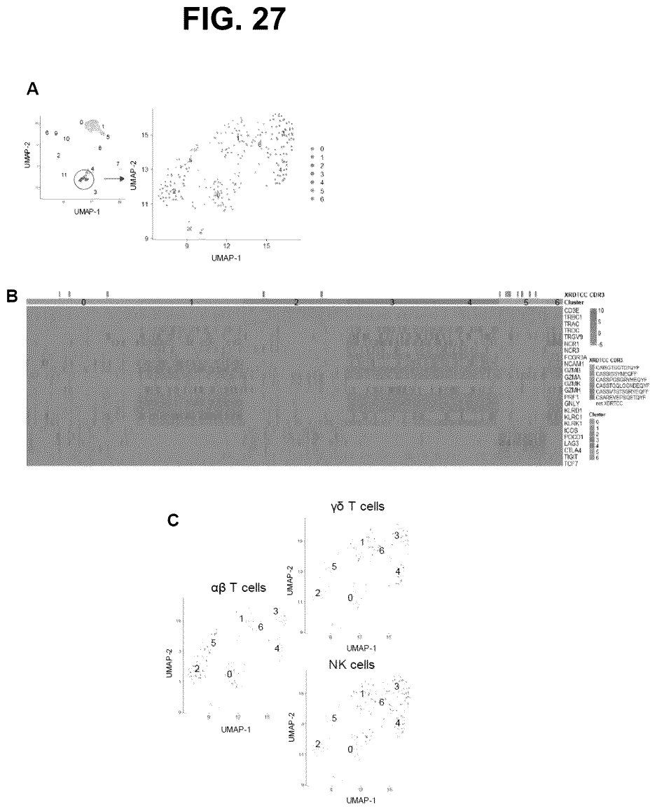

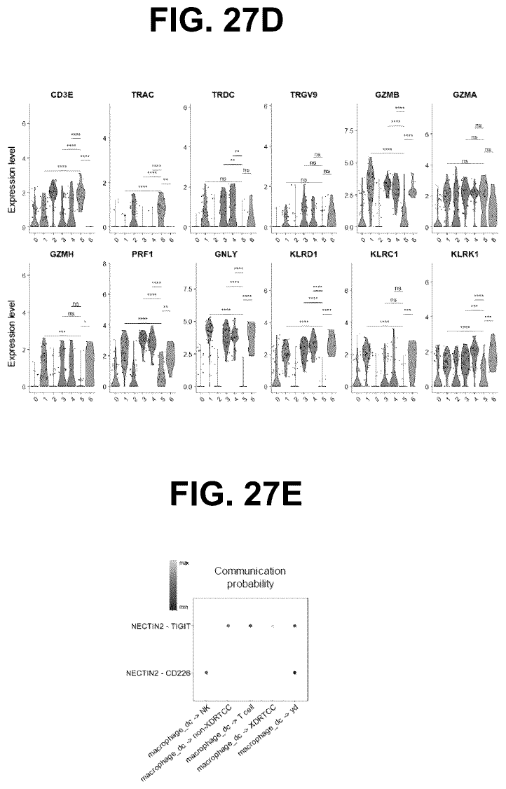

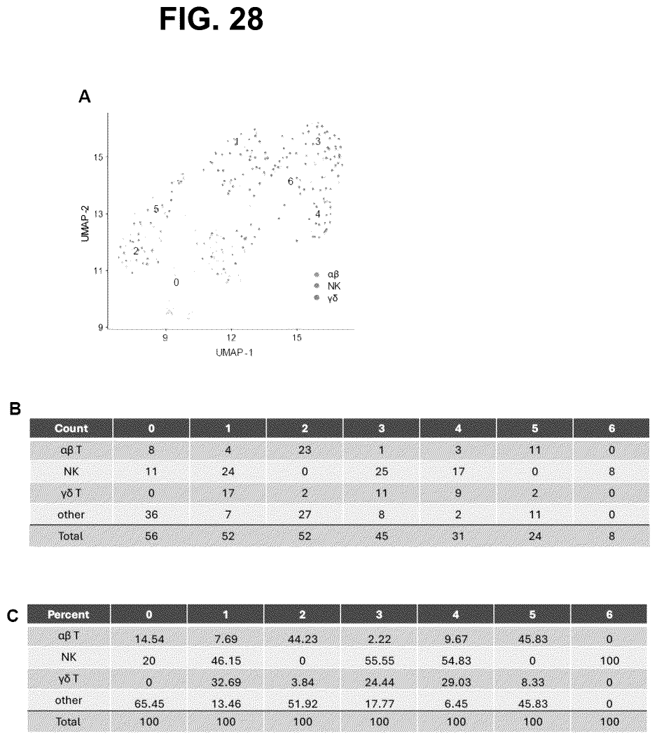

To dissect cluster 3 at greater resolution, this subset of cells (FIG. 27A) was re-clustered. The majority of XDRTCCs mapped onto Cluster 5 of this new UMAP (FIG. 27B). Cluster 5 included some effector genes such as GZMA, GZMB and PRF1 as well as the costimulatory molecule ICOS and some coinhibitory molecules, including PDCD1, LAG3, CTLA4, and TIGIT (FIG. 27B). Clusters 1, 3, 4, and 6 all demonstrated high expression of cytotoxic markers including GZMB, GZMA, GZMH, PRF1, and GNLY (FIG. 27B). They also expressed high levels of TRDC and TRGV9, consistent with a γδ T cell phenotype (FIG. 27B). Indeed, by transcriptional profiles, the innate and adaptive cells clustered separately in the analysis, with αβ T cells (CD3E⁺ TRBC1⁺ TRDC^{neg}) mainly in Clusters 0, 2, and 5 (FIG. 27C and Table 4), where XDRTCCs were located (FIG. 27B). In contrast, γδ T cells (CD3E⁺ TRDC⁺) were detected mainly in Clusters 1, 3, and 4 and NK cells (CD3E^{neg} and FCGR3A⁺ or NCAM1⁺) were detected mainly in Clusters 0, 1, 3, 4, and 6 (FIG. 27C and FIGS. 28A-C).

Given that γδ T cells also express unique TCRs, their V gene usage were examined. γδ T cells (CD3E+ TRDC+) identified in POD14L and in POD33K (FIG. 29A) showed divergent TRGV gene usage (FIG. 29B), suggesting new recruitment of γδ T cells at POD33 rather than migration from POD14L. Indeed, γδ T cells at POD33 predominantly expressed TRGV9, consistent with peripheral blood origin. Furthermore, those in the POD33 kidney were phenotypically distinct, with elevated cytotoxic gene expression, including GZMB, GZMA, PRF1 and GNLY (FIG. 29C).

To compare the RNA profile of γδ and NK cells against αβ T cells, the expression of a T cell cytotoxicity pathway from MSigDB was first examined. Indeed, the clusters containing γδ and NK cells tended to have higher expression of the signature than those containing αβ T cells (FIG. 29D). To compare their cytotoxicity in detail, the expression of cytotoxic genes was assayed. Clusters 1, 3, 4, and 6 containing γδ T cells and NK cells frequently expressed significantly higher levels of cytotoxic markers including GZMB, GZMH, PRF1, and GNLY than cluster 5 containing the majority of αβ XDRTCCs (FIG. 27D). GZMA levels were similar between clusters containing αβ as well as γδ and NK cells (FIG. 27D).

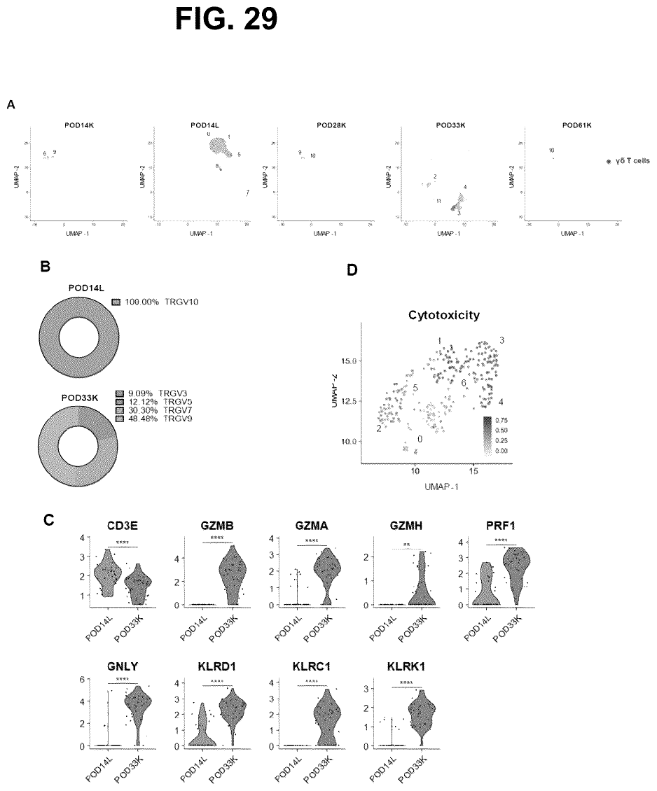

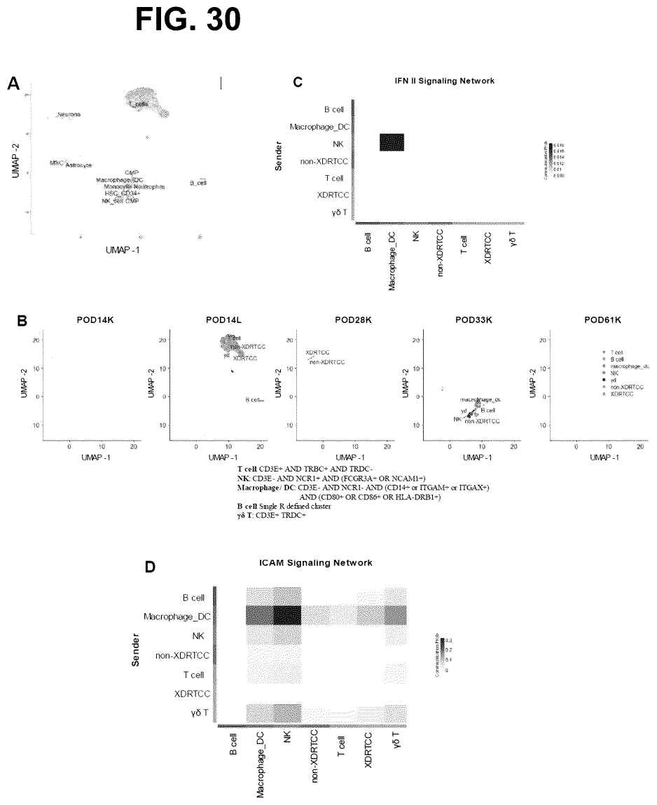

The CellChat package algorithm was used to identify the likely cell-cell interactions of the different lymphocyte types. For the analysis, the same cell labelling strategy shown in FIG. 22 was used, which was in agreement with SingleR predictions (FIGS. 30A-B). CellChat predicted cell-cell interactions including the NECTIN2 pathway, which has two ligands mediating opposite effects on lymphocytes: TIGIT, which inhibits activation and CD226, which promotes cytotoxicity. Interestingly, macrophages and dendritic cells (FIGS. 30A-B) were predicted to interact with XDRTCCs via TIGIT but with NK and γδ T cells via CD226, suggesting they activated cytotoxicity in innate lymphocytes (FIG. 27E). CellChat analysis further indicated that NK cells are likely a high producer of IFN-γ for macrophages and dendritic cells (FIG. 30C), and these APC populations were predicted to express ICAM pathway adhesion molecules (FIG. 30D) that strongly bind to their receptors on NK and γδ T cells. Taken together, these data suggest that during an episode of acute rejection, innate lymphocytes as well as αβ T cells contribute significant cytotoxic effector function.

### Discussion

The present disclosure provides a first instance of xenogeneic donor-reactive T cell identification and tracking following a pig to human organ transplant. The data described herein demonstrated marked and specific expansion of XDRTCCs in the circulation and their detection among leukocytes obtained from kidney biopsy specimens during AMR and from a lymphocyte collection prior to rejection, suggesting that they may contribute to or even initiate AMR. The disclosure also provides evidence for recipient APC infiltration and activation within the xenograft during rejection as well as activated γδ T cells and NK cells with effector function. These studies provide novel insights into human anti-pig responses in vivo and demonstrate that improved suppression of these pathways or tolerance induction may enhance the ultimate success of clinical xenotransplantation.

In the past two years, the first pig-to-human kidney and heart xenotransplants have been performed in decedents and living patients. Histological analyses of short-term (up to 7 days) pig kidneys transplanted into deceased human recipients have not revealed evidence for rejection. However, immune profiling of a 54-hour decedent study revealed microvascular inflammation suggestive of AMR, a transcriptional profile consistent with antibody-mediated injury, the presence of natural killer cells and monocyte/macrophage activation, without markers of T cell-mediated rejection. This study is the first to characterize the donor-specific T cell repertoire, building on an established and validated method in human allograft recipients. The study also is the first to implicate these T cells in xenograft rejection in a human. The naïve human anti-pig xenoresponse, in contrast to the alloresponse, also includes strong indirect responses. This difference likely reflects greater numbers of antigenic disparities at the peptide level due to more extensive protein polymorphisms between pigs and humans than between allogeneic humans. Extending the high-throughput TCRB CDR3 sequencing approach to an analysis of the human anti-pig xenoresponse revealed that, like alloresponses, the diversity of xenoreactive T cells is lower than that of unstimulated T cells and is in the same range as direct alloresponses. The potent primary indirect xenoresponse shows similar diversity as the direct xenoresponse, consistent with the notion that the extensive protein/peptide non-homology between pigs and humans causes strong and diverse indirect xenoresponses. Clonotypic analysis revealed very minimal overlap between the sequences identified for the direct versus indirect CD4 or CD8 xenoresponse, indicating recognition of distinct specificities in each type of assay and arguing against semi-direct presentation as an explanation for the potent indirect response.

Clonotypic analyses of the decedent anti-donor pig response allowed us to identify XDRTCCs in the recipient and track them in the post-transplant circulation and the graft. While the number of unique XDRTCCs identified pre-transplant was limited due to a technical problem with clumping among cryopreserved decedent cells, donor-reactive CD4 and CD8 T cell clones were identified directly and indirectly. These numbers were augmented by identifying additional XDRTCCs in post-transplant MLRs. Post-transplant tracking of these XDRTCCs in the circulation revealed progressive and marked expansion, including both CD4 and CD8 subsets. The most striking expansion was observed for CD8 XDRTCCs, among which a single, directly-xenoreactive clone represented >20% of the repertoire by POD49. While this expansion of XDRTCCs occurred on a background of anti-thymocyte globulin-induced T cell lymphopenia which also led to the expansion of some non-XDRTCCs, the relative expansion of XDRTCCs was markedly greater (>150-fold by POD49). These results demonstrate a marked in vivo T cell response to the donor, a conclusion that is reinforced by the high level of clonal “convergence” for this subset, where increased numbers of unique clones encoding the same XDRTCC CDR3s were expanded after the transplant.

The same circulating immunodominant CD8 XDRTCCs were also found in the graft. They were almost all directly xenoreactive, in concordance with the circulating XDTRCC clones, and the infiltrating CD8 cells expressed an effector transcriptional profile, including cytotoxic genes and IFN-γ. A perinephric lymphocele collected on POD14, prior to rejection, contained directly in addition to indirectly xenoreactive CD4 clones as well as both directly and indirectly xenoreactive CD8 clones, collectively suggesting that activation of XDRTCCs occurred in the lymphoid tissues and mainly CD8 effector cells infiltrated the graft. Some of the infiltrating αβ CD8 XDRTCCs also expressed genes associated with exhaustion, perhaps reflecting the strong stimulation associated with their marked expansion in the decedent. Overall, the expansion of circulating XDRTCCs and their selective appearance only in the rejection biopsies suggests that the immunosuppression used was insufficient to fully suppress the anti-donor xenoresponse and raises the possibility that XDRTCCs were the initiators of innate immune infiltration, APC activation and AMR in the xenograft kidney. Previous pig-to-decedent human kidney transplants primarily reported innate and not adaptive immune activation, likely because their shorter time scale did not allow observation of the adaptive response.

The present disclosure describes the development of a porcine thymic transplantation for the purpose of xenograft tolerance induction and demonstrates its capacity to tolerize xenogeneic T cells in immunocompetent subjects. specifically, a pig-to-non-human primate transplantation was used to generate composite thymokidney grafts in prospective porcine donors, and was utilized in the decedent experiment, or by transplanting primarily vascularized porcine thymic lobes. While donor-specific hyporesponsiveness was demonstrated in vitro in some non-human primate studies, immunosuppression has not been withdrawn. As such, prior studies did not establish immune tolerance.

Recipient thymectomy was performed because studies in the murine model showed that the absence of a native thymus as well as exhaustive depletion of pre-existing T cells was essential for the engraftment and tolerance-inducing capacity of porcine thymic xenografts. In contrast, the decedent transplant recipient was not thymectomized. However, anti-donor MLR responses following the transplant revealed gradually declining anti-pig responses with no change in anti-human responses. Thus, despite the expansion of XDRTCCs and their presence in the rejecting xenograft, and the detection of only minimal porcine thymic tissue at the time of experimental termination it is possible that the T cell compartment was rendered partially tolerant of the source pig, perhaps due to regulatory T cell development in the pig thymus.

In addition to a role for T cells in kidney xenograft rejection, these studies also implicate two innate components of the immune system, namely NK cells and γδ T cells, both of which showed strong activation/effector signatures within the rejecting xenograft biopsy. NK cells are thought to play an important role in xenotransplantation, given the lack of NK cell inhibiting surface proteins (e.g., pig MHCI, KIR receptors) in pigs. NK cell gene signatures were prominent in subclinical humoral rejections affecting mainly the glomeruli in shorter-term pig-to-decedent human transplants. Both antibody-dependent cellular cytotoxicity (ADCC) and direct cytotoxic activity induced by cytokines produced by xenoreactive T cells may play significant roles, consistent with the transcriptional profile of cell-mediated cytotoxicity among NK cells that was detected in the rejection specimen. In vitro studies of human anti-pig MLRs have demonstrated NK cell activation in the presence of exogenous IL-2 and/or whole PBMCs, presumably reflecting a requirement for cytokines produced by T cells.

A marked activation/effector transcriptional profile for γδ T cells was also observed. γδ T cells were prominent among the lymphocytes isolated from the rejecting biopsy specimen. The role of γδ T cells in xenotransplantation has not been well-studied, though they have previously been shown to play a critical role in rejection in rat to mouse bone marrow transplantation. Further studies of the conditions and molecular interactions that involve γδ T cells in xenograft rejection are warranted. It is noteworthy that the immunosuppressive agents used in the study described herein (Table 1) were geared toward suppression of the adaptive and not the innate immune response. Further studies to understand the extent to which innate cellular components of the xenoresponse are dependent on T cell responses can determine the need only for improved T cell suppression and tolerance versus a need to separately target innate cellular immune responses.

### Example 10: Materials and Methods

### Experimental Model and Transplantation Protocol

A thymokidney was transplanted from an α-GalKO donor pig (Revivicor) to a decedent recipient following bilateral native kidney nephrectomy. The source pig was a female, 199-day-old at time of procurement, and weighed 80.7 kg. The brain-dead recipient was a 56-year-old male, weighing 79.5 kg with stage IV glioblastoma, making him unsuitable for organ donation. The total ischemia time was 4 h and 45 minutes. The immunosuppression regimen is shown in Table 1. Five months before transplantation, autologous thymus tissue was implanted under the source pig's kidney capsule. The native kidneys were subsequently removed, and one thymokidney was transplanted orthotopically to the decedent.

### Patient Sample Collection

Blood, lymphocele, lymph node tissues and kidney biopsies were collected at different timepoints pre-transplant and on PODs-3, 0, 7, 14, 21, 28, 33, 35, 42, 45, 49, 56, and 61.

### Sample Processing

PBMCs were obtained from blood samples at each time point using Ficoll (Sigma-Aldrich, catalogue number 10771-500 ml) density gradient centrifugation. Isolated PBMCs were washed with phosphate buffered saline (PBS, Cytvia, catalogue number SH30256.02) and red blood cells (RBCs) were lysed by ACK lysing buffer (Gibco, catalogue number A10492-02) for 3 minutes, followed by another PBS wash. PBMCs were cryopreserved in liquid nitrogen in freezing media containing human AB serum (Gem Cell, catalogue number 100512) and 10% DMSO (Sigma Aldrich, D2650-100 ml).

Kidney core needle biopsies were collected on various PODs, including 14, 28, 33 and 61. The kidney tissues were digested using the Gentle MACS kit (Multi Tissue Dissociation Kit 1, Miltenyi Biotec, catalogue number 130-110-201) according to the manufacturer's instructions and human leukocytes were isolated via human CD45⁺ FACS sorting (BD, Influx). Lymph nodes were gently crushed, strained through 70 μm filters and washed twice with PBS and single-cell suspensions were prepared. Additionally, a perinephric lymphocele drainage sample was obtained on POD14. Leukocytes from the lymphocele sample were isolated using a Ficoll density gradient.

### Direct and Indirect Mixed Lymphocyte Reactions (MLR)

We developed direct and indirect MLR assays using healthy control T cells as responders. For direct MLR, porcine DCs were differentiated from PBMCs and used as stimulators. Twenty-five million porcine PBMCs were plated in MLR medium (AIM-V supplemented with 5% AB heat-inactivated human AB serum, 0.01 M HEPES, and 50 mM 2-mercaptoethanol, 1% penicillin/streptomycin) at a concentration of 2×10⁶ cells/mL. After 12 hours, non-adherent cells were harvested and discarded, and the medium was replaced with fresh medium containing 50 ng/ml of porcine granulocyte-macrophage colony-stimulating factor (pGM-CSF, bioteche, catalogue number 711-PG) and 20 ng/mL porcine interleukin-4 (pIL-4, bioteche, catalogue number 654-p4-025/CF). On day 3, 1 mL of medium containing 10× concentrated porcine cytokines (500 ng/mL pGM-CSF, 200 ng/mL pIL-4) was added. On day 4 (12 hours before harvesting) DCs were matured/activated by adding 1 μg/mL of LPS (Milipore Sigma, catalogue number L2387). On day 5, DCs were detached by agitation with cold MACS buffer. Pig DCs were then resuspended in PBS and labelled with BD Horizon Violet Proliferation Dye 450 (VD450, catalogue number 562158). After labelling, cells were washed twice, resuspended in MLR medium at 2×10⁶ cells/mL, and irradiated at 30 to 35 Gy, and subsequently co-cultured with responder cells. The responders comprised human CD3⁺ T cells isolated using a CD3-negative (to keep the T cells untouched) MACS sorting kit (Pan T Cell Isolation Kit II, Miltenyi Biotec, catalogue number 130-096-535) and labelled with CFSE (CellTrace CFSE Proliferation Kit, Invitrogen, catalogue number C34554). These T cells were resuspended in PBS at 1×10⁶ cells/mL, labelled with CFSE at a concentration of 0.2 to 0.5 μM, washed three times, and resuspended in MLR medium. 96-well round bottom plates were used with 200,000 responder T cells and 20,000 pig DC stimulators per well (10:1 ratio) (total well volume, 200 μl). MLR cultures were incubated at 37 °C for 6 days. CFSE^{low} (proliferated) CD4 and CD8 T cells were FACS sorted and DNA was extracted (Qia gen, catalogue number 69504) and deep TCRB sequencing was carried out at Adaptive Biotech. Detailed information on the materials, reagents, and antibodies used in this study is provided in Tables 5 and 6.

For the indirect MLR, macrophages were differentiated from human monocytes and loaded with apoptotic pig cells generated as described below, serving as stimulators. CD14⁺ monocytes were isolated from 30 million human PBMCs using a MACS negative selection kit (Classical Monocyte Isolation Kit, Miltenyi Biotec, Cat number 130-117-337). Subsequently, they were plated in IMDM (Gibco, catalogue umber 12440-053) supplemented with 10% human AB serum (100,000 cell/cm²). After 12 hours, non-adherent cells were removed, and fresh media containing 50 ng/ml M-CSF (PeproTech, catalogue number 300-25) was added. On day 4, the media, now including M-CSF (50 ng/ml) and IL-4 (20 ng/ml, PeproTech, catalogue number 200-04), was refreshed. On day 6, 3 million differentiated macrophages were harvested and co-cultured with 9 million apoptotic (100 Gy-irradiated) porcine PBMCs in 6 wells of a 6-well plate (1:3 ratio, 150,000 macrophages versus 450,000 apoptotic porcine PBMC in each well). After 12 hours, IFN-γ (50 ng/ml, PeproTech, catalogue number 300-02) and LPS (50 ng/μl) were added to the media. Twelve hours later, activated and antigen-loaded macrophages were harvested and purified again by CD14-negative MACS sorting. After MACS sorting, 500,000 cells were retrieved and stained by VD450 and used as stimulators (see above). The responder cells were stained by CFSE and cocultured with activated, antigen-loaded, and VD450 stained macrophages at a ratio of 5 to 1 (200,000 responders and 40,000 stimulators). After 6 days at 37 °C, CD4 and CD8 CFSE^{low} proliferated cells were isolated, DNA was extracted, and deep TCRB CDR3 sequencing was carried out at Adaptive Biotech. Other-ratios of responders and stimulators were tested, and the greatest proliferation was seen at a 5 to 1 ratio.

### Direct and Indirect MLRs for Decedent Recipient Against Donor Pig

After establishing our direct and indirect MLR assays, we employed this method in our decedent study. 150 mL blood samples were collected from both the donor pig and the decedent recipient one day prior to the procedure. On PODs 28 and 49 we received 50 and 22.5 ml of blood (FIG. 13A). PBMCs were isolated from blood samples and preserved in cryopreservation media for use in subsequent experiments. During the PBMC preparation and thawing processes, an unexpected issue was observed: the cells demonstrated unusual clumpiness, resulting in a yield of PBMCs that was significantly lower than anticipated. Consequently, while we were able to set up both direct and indirect MLR with low total cell numbers for the pre-transplant time point, we were able to only do direct MLR for POD28 and total MLR (recipient PBMC as responder versus donor pig PBMCs as stimulator) for POD49. Due to the low number of cells and hence clones obtained from our pre-transplant MLR, we also used T cells isolated from pre-transplant lymph node as responders versus donor pig DC as stimulators in a direct MLR. High throughput TCR sequencing was done on sorted unstimulated CD4 and CD8 T cells and CD4 CFSE^{low} and CD8 CFSE^{low} T cells after stimulation in the direct and indirect MLR assays described above.

### Bulk TCRB CDR3 Sequencing

Deep TCRB CDR3 sequencing was performed on genomic DNA extracted from CD4 CFSE^{low} and CD8 CFSE^{low} cells pre- and post-transplantation. Genomic DNA from unsorted and unstimulated PBMCs samples was also sent for bulk TCRB CDR3 sequencing. Additionally, sorted CD4⁺ and CD8⁺ sorted cells from post-transplant peripheral blood samples obtained at POD28, 33, and 49 were sequenced. The extracted genomic DNA was sent to Adaptive Biotechnologies for TCRB chain CDR3 sequencing according to their immunoSEQ Platform protocols, which include standards for sequencing and error correction.

### Sample Processing

We loaded a table of the CDR3 amino acid and nucleotide sequences of the TCRB sequences into RStudio (version 4.3.1) and screened them for known lab contaminants at the nucleotide level. The few contaminants found were removed. The sequences were further processed using a modified ‘resolve ambiguous’ function with an ambiguity ratio of 5. Using this function, clones present in both CD4⁺ and CD8⁺ populations of the same sample, with a frequency in one population at least 5 times higher than in the other, were removed from the population with the lower frequency. A slight modification was made to the ‘resolveambiguous’ function to preserve putative sequences present in both populations at an ambiguity ratio lower than 5. These clones had to meet a minimum frequency threshold of 1E-5 in both CD4⁺ and CD8⁺ populations to be preserved, which reduced the possibility of cross-contamination or sorting error.

XDRTCCs were then identified at the amino acid levels. Unique sequences from CD4⁺ and CD8⁺ CFSE^{Low} populations were individually compared to their respective abundance in the pre-transplant peripheral repertoire. To be defined as a XDRTCC, individual clones needed to have an abundance in their respective CFSE^{Low} repertoires that was at least twice their abundance in the pre-transplant unstimulated repertoire (Table 2). The remaining XDRTCCs from all the different MLRs were then pooled together according to their CD4⁺ and CD8⁺ identity and combined into a single repertoire to facilitate analysis and tracking. Each clone was only counted once in the pooled repertoires.

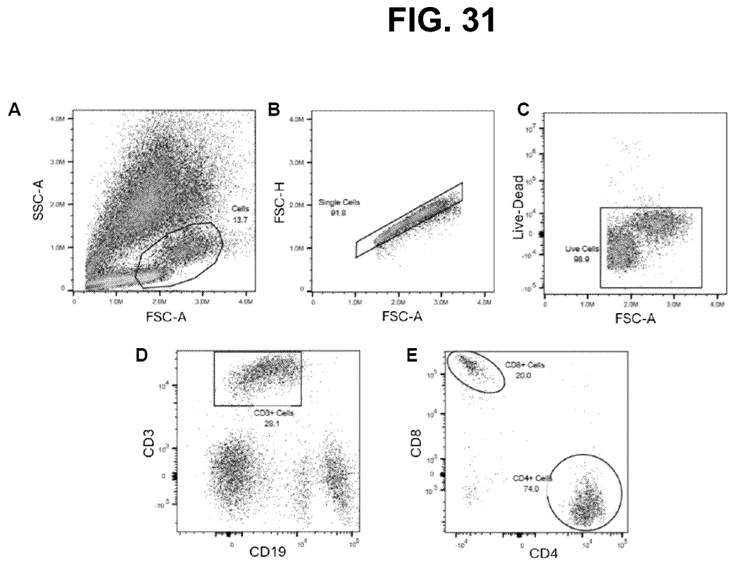

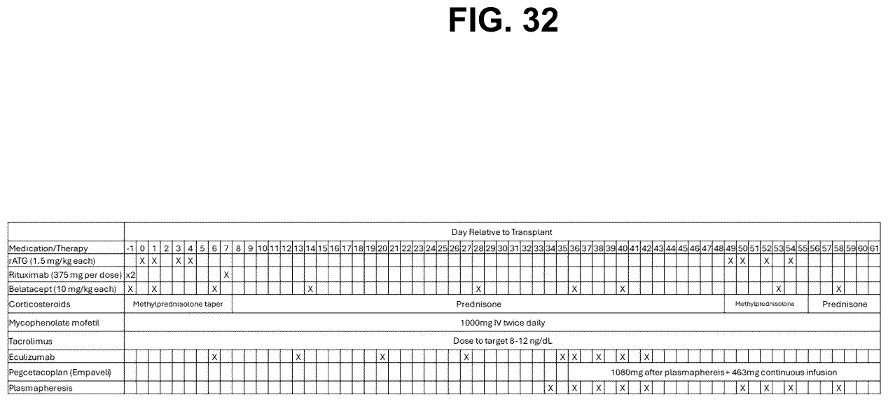

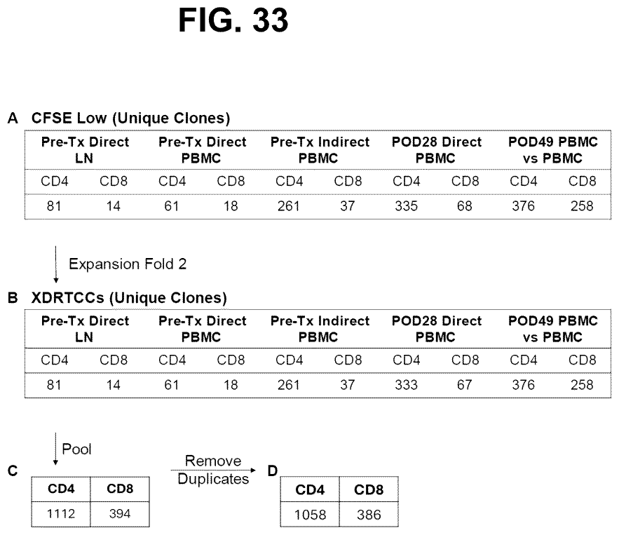

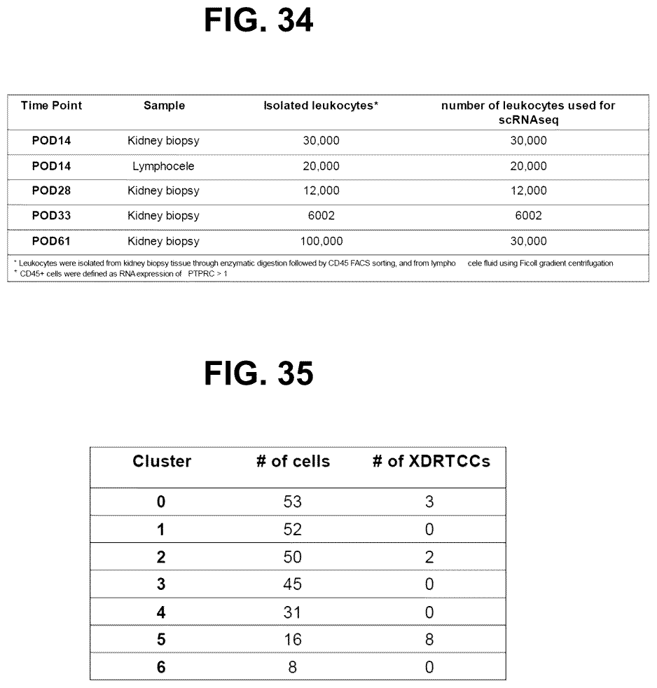

Non-xenogeneic donor-reactive T cell clones (non-XDRTCCs) were defined by exclusion, starting with the pre-transplant unsorted peripheral blood repertoire. Specifically, CD4 clones detected in the pre-transplant unsorted, unstimulated peripheral blood repertoire but not in the CD4⁺ CFSE^{Low} populations constituted the CD4 non-XDRTCC repertoire. Similarly, non-overlapping CD8 clones between the pre-transplant unsorted, unstimulated peripheral blood T cell repertoire and the CD8⁺ CFSE^{Low} populations constituted the CD8 non-XDRTCC repertoire. Although the poor PBMC yields precluded separate sorting of unstimulated CD4⁺ and CD8⁺ T cells pre-transplant, many of the same clones were later mapped to post-transplant sorted populations, allowing us to assign their CD4⁺ or CD8⁺ identity. The total number of CD4⁺ and CD8⁺ templates in the unsorted pre-transplant peripheral population was estimated using pre-transplant flow cytometric data, which showed that 74.0% of CD3⁺ T cells were CD4⁺ and 20.0% were CD8⁺ (FIG. 31). This allowed us to estimate the number of CD4⁺ and CD8⁺ non-XDRTCCs.

The CD4⁺ and CD8⁺ XDRTCC and non-XDRTCC repertoires were then mapped onto post-transplant CD4⁺ and CD8⁺ sorted peripheral populations. We calculated clonal abundance, or frequency, defined as the total number of templates associated with a mappable clone divided by the total number of templates, in these populations (FIGS. 16A-B).

Since XDRTCCs and non-XDRTCCs were identified by amino acid sequence, individual clones could have more than one synonymous nucleotide sequence associated with them (FIG. 17E). A clonal convergence analysis was done comparing the average number of unique TCR nucleotide sequences associated with the same amino acid sequence for XDRTCCs and non-XDRTCCs found in the peripheral blood.

The computations were done in RStudio, and most plots were produced in GraphPad Prism. Other R packages used included immunarch and eulerr.

### Single-Cell RNA and TCR Sequencing

Kidney biopsy and lymphocele samples were processed as described above and sorted CD45⁺ immune cells were submitted to the Columbia Human Immune Monitoring Core for standard single-cell RNA using 10× GEM Chromium 5′ Ver2. TCR libraries were prepared from the same samples. Gene expression libraries were sequenced at a minimum depth of 25,000 reads per cell and TCR sequencing at a depth of 5,000 reads per cell. FACS sorted CD4 and CD8 CFSE^{low} cells in the healthy control MLR assays were loaded and submitted according to the same protocol.

Sample Processing CellRanger (v.6.1.2 pipeline) was used to pre-process sequenced reads against reference GRCh38-2020-A (from Cellranger) and Sscrofa11.1 (from Ensembl). A hybrid human and pig reference sequence set were generated using Cellranger's mkref function. The hybrid reference was used to map the transcripts all cells. The barcodes of cells with significant pig transcript counts were identified. The human-only reference set was then used to map the transcripts of all cells. Any overlapping barcodes of the previously identified pig cells were removed. Downstream analysis of the filtered UMI expression profile was completed using R (v.4.4.0) and Seurat (v.5.0.3). CellRanger outputs were assembled into the count matrix using and cells with at least 500 unique features, between 1000 to 25000 UMIs, and mitochondrial RNA <15% were retained for further analysis. Features expressed in less than 50 cells were also removed. Data were then normalized using SCTransform and data from different samples were integrated using Seurat's CCA based workflow. Cells were assigned cell cycle scores using S and G2M phase genes (from cc.genes included in the Seurat package). Data were normalized and scaled with S and G2M scores regressed out. Thirty principal components were used to construct a K nearest neighbors graph and clusters identified by the shared nearest neighbor (SNN) algorithm. Clusters were visualized using the UMAP algorithm. Differential expression was done using Seurat's implementation of the Wilcoxon rank sum test with FindMarkers. Violin plots were generated in Seurat. Heatmaps were generated using the pheatmap library. Pathway analysis was done using Seurat's DEenrichrPlot modified to use the top differentially expressed genes by log fold change. Cytotoxicity gene signature was conducted using the gene set M12145 and AddModuleScore. Cell-cell interaction predictions were generated using CellChat package. Reference based cell labeling using the Human Primary Cell Atlas was conducted using SingleR.

### Statistical Methods

Analyses were conducted in Graphpad Prism 9.0 or 10.0 (GraphPad Software, USA) as well as RStudio (version v.4.4.0).

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

What is claimed is:

1. A method of inducing a transplant tolerance, the method comprising:
(a) macerating a thymic tissue obtained from a donor animal;
(b) injecting the macerated thymic tissue into a kidney capsule or a lung of the donor animal or a first recipient, wherein the donor animal or the first recipient is thymectomized prior to the injection;
(c) maturing the injected macerated thymic tissue into a composite renal subcapsular thymic lymphoid tissue or a composite pulmonary thymic lymphoid tissue; and
(d) generating a composite thymo-kidney organ or a composite thymo-lung organ.

2. A method of inducing a transplant tolerance, the method comprising:
(a) macerating a thymic tissue obtained from a donor animal;
(b) culturing the macerated thymic tissue in a culture regimen for about 1 day to about 30 days;
(c) injecting the macerated thymic tissue into a kidney capsule, a lung, or a quadriceps muscle of the donor animal or a first recipient, wherein the donor animal or the first recipient is thymectomized prior to the injection;
(d) maturing the injected macerated thymic tissue into a composite renal subcapsular thymic lymphoid tissue, a composite pulmonary thymic lymphoid tissue, or a composite musculo-thymic lymphoid tissue; and
(e) generating a composite thymo-kidney organ, a composite thymo-lung organ, or a thymo-muscle organ.

3. The method of claim 1, wherein the macerated thymic tissue is injected:
(a) subcapsularly into the kidney capsule of the donor animal or the first recipient; or
(b) into a lung parenchyma or a lung pleura of the donor animal or the first recipient.

4. The method of claim 1 further comprising transplanting the composite thymus-kidney organ or the composite thymo-lung organ in a subject in need of a kidney or a lung transplant.

5. The method of claim 4, wherein the composite thymus-kidney organ or the composite thymo-lung organ induces thymopoiesis in the subject within about 1 month, about 2 months, about 3 months, about 4 months, about 5 months, about 6 months, about 7 months, about 8 months, about 9 months, about 10 months, about 11 months, or about 12 months.

6. The method of claim 2, wherein the macerated thymic tissue is injected into the donor animal and the composite thymo-kidney organ is an autologous composite thymo-kidney organ.

7. The method of claim 1, wherein the donor animal is partially thymectomized using a blunt and sharp dissection.

8. The method of claim 1, wherein macerating comprises:
(a) slicing the donor thymic tissue into about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.2 mm, about 1.3 mm, about 1.4 mm, or about 1.5 mm slice; or
(b) slicing the donor thymic tissue into about 1 inch long and a quarter inch diameter.

9. The method of claim 3, wherein the subcapsular injection is performed by a method comprising the steps of:
(a) placing the donor animal in a lateral position;
(b) introducing a horizontal or vertical incision extending from the last rib into the paralumbar fossa and over the kidney;
(c) mobilizing the caudal pole and lateral aspect of the kidney;
(d) exposing the kidney within a Gerota's fascia;
(e) introducing a small incision in the kidney capsule;
(f) separating the renal capsule from the renal cortex to generate a pocket;
(g) injecting the macerated thymic tissue into the pocket;
(h) closing the kidney capsule, optionally wherein the kidney capsule is closed with a Prolene 6-0 suture, or the kidney capsule incision is cauterized; and
(i) closing the horizontal or the lateral incision.

10. The method of claim 9, wherein the horizontal or vertical incision is about 5 cm to about 8 cm.

11. The method of claim 3, wherein:
(a) the macerated thymic tissue is injected subcapsularly toward the cranial pole of the kidney capsule; and/or
(b) the injected macerated thymic tissue covers one surface of the caudal renal pole.

12. The method of claim 3, wherein the subcapsular injection is performed using a silicone catheter, a blunted needle, a modified pipette, or a robot.

13. The method of claim 2, wherein the macerated thymic tissue is cultured for about 5 to about 7 days, about 5 to about 12 days, about 5 to about 9 days, about 10 to about 20 days, about 5 to about 12 days, about 5 to about 15 days, or about 10 to about 20 days.

14. The method of claim 2, wherein culturing comprises contacting the thymus tissue with an agent selected from the group consisting of 2-deoxyguanosine (2DG), cyclosporine A, hydrocortisone, notch inhibitors, ABT-737, and 2D glucose, or a combination of 2 or more thereof.

15. The method of claim 14, wherein culturing comprises:
(a) contacting the thymic tissue with 2-DG;
(b) contacting the thymic tissue with about 1 nM to about 500 nM 2-DG, about 25 nM to about 250 nM 2-DG, about 50 nM to about 150 nM 2-DG, about 50 nM to about 100 nM 2-DG, about 100 nM to about 300 nM 2-DG, or about 250 nM to about 500 nM 2-DG for about 2 hours to about 24 hours; or
(c) contacting the thymic tissue with about 100 nM 2-DG for about 15 hours.

16. The method of claim 2, wherein:
(a) the culture regimen depletes the thymus tissue of donor thymocytes; and/or maintains thymic stromal components; or
(b) culturing comprises suspending the thymic tissue in an extracellular matrix composition or a biomaterial prior to injecting the macerated thymic tissue into the kidney capsule, the lung, or the quadriceps muscle of the donor animal or the first recipient.

17. The method of claim 16, wherein the extracellular matrix composition comprises a collagen matrix, a laminin matrix, a fibronectin matrix, a collagen/laminin matrix, or Matrigel™.

18. The method of claim 4, wherein culturing further comprises co-culturing the thymic tissue with isolated thymic epithelial cells obtained from a subject to be transplanted, optionally wherein the thymic tissue and the isolated thymic epithelial cells are injected into the kidney capsule, the lung, or the quadriceps muscle of the donor animal or the first recipient.

19. The method of claim 1, wherein the donor is a pig.

20. The method of claim 19, wherein the pig is a transgenic pig that:
(a) lacks expression of a functional gene selected from the group consisting of an alpha 1,3 galactosyltransferase, a growth hormone receptor (GHR), a β-1,4-N-acetyl-galactosaminyltransferase 2 (β4GalNT2) and a cytidine monophosphate-N-acetylneuraminic acid hydroxylase (CMAH), or a combination thereof; and
(b) expresses at least six exogenous transgenes selected from an anticoagulant transgene, a cytoprotective transgene, an immunosuppressant transgene, or a complement inhibitor transgene, or a combination thereof.
